SEQUENCE LISTING (2 sequences)

SEQ ID NO: 1 - DNA, 20 bp, synthetic construct
  source 1..20: mol_type = other DNA
tgccccaattctggagtagg

SEQ ID NO: 2 - DNA, 19 bp, synthetic construct
  source 1..19: mol_type = other DNA
cggcacgtggacacagatt

(19) United States
(12) Patent Application Publication
(10) Pub. No.: US 2023/0042830 A1
(43) Pub. Date: Feb. 9, 2023

(54) COMPOSITIONS AND METHODS FOR GENERATION OF RETINAL GANGLION CELLS FROM INDUCIBLE PLURIPOTENT STEM CELLS FOR THE TREATMENT OF PROGRESSIVE OPTIC NEUROPATHIES, INCLUDING GLAUCOMA

(71) Applicant: THE TRUSTEES OF THE UNIVERSITY OF PENNSYLVANIA, Philadelphia, PA (US)

(72) Inventors: Venkata Ramana Murthy Chavali, Malvern, PA (US); Jason Adam Mills, Chester Springs, PA (US)

(73) Assignee: THE TRUSTEES OF THE UNIVERSITY OF PENNSYLVANIA, Philadelphia, PA (US)

(21) Appl. No.: 17/867,335

(22) Filed: Jul. 18, 2022

Related U.S. Application Data
(60) Provisional application No. 63/222,789, filed on Jul. 16, 2021.

(51) Int. Cl. C12N 5/079; A61P 27/02

(52) U.S. Cl. CPC C12N 5/0621; A61P 27/02; C12N 2501/115; C12N 2501/41; C12N 2506/45; C12N 2501/13; C12N 2501/105

(57) ABSTRACT

Compositions and methods for inducing pluripotent stem cells into retinal ganglion cells for administration to a subject for treating progressive optic neuropathies, thereby alleviating symptoms of such disorders including glaucoma.

### CROSS REFERENCE TO RELATED APPLICATION

This application claims priority to U.S. Provisional Application No. 63/222,789, filed on Jul. 16, 2021, the entire disclosure of which is incorporated herein by reference as though set forth in full.

### GRANT STATEMENT

This invention was made with government support under EY023557-01 awarded by the National Institutes of Health. The government has certain rights in the invention.

### REFERENCE TO AN ELECTRONIC SEQUENCE LISTING

The contents of the electronic sequence listing (UPNK-110-US01_SEQLIST.xml, Size: 2,955 bytes; and Date of Creation: Oct. 3, 2022) is herein incorporated by reference in its entirety.

### FIELD OF THE INVENTION

This invention relates to fields of progressive optic neuropathies and generation of desired cell types via directed differentiation of iPSCs. More specifically, the invention provides compositions and methods for the treatment of eye-related disorders, such as Glaucoma, Optic Neuritis and retinal degenerations via the administration of retinal ganglion cells (RGCs) and/or retinal progenitor cells (RPCs), thereby alleviating symptoms of such disorders.

### Background of the Invention

Several publications and patent documents are cited throughout the specification in order to describe the state of the art to which this invention pertains. Each of these citations is incorporated herein by reference as though set forth in full.

Glaucoma encompasses a heterogenous group of optic neuropathies, characterized by their progressive structural and functional deterioration of the optic nerve resulting in permanent visual field loss. This degeneration is due to the death of RGCs, with subsequent visual field loss. Primary open-angle glaucoma (POAG), the most common form of glaucoma, is characterized by chronic and progressive optic nerve degeneration and corresponding visual field deficits in the presence of an open and normal iridocorneal chamber angle. In 2020, about 52.68 million individuals, aged 40-80, were diagnosed with POAG. By 2040, that number is expected to increase to 79.76 million (3, 4), making POAG the leading cause of irreversible blindness worldwide (5, 6). In the United States alone, 3 million Americans are projected to have glaucoma and this number is estimated to double by the year 2050. Furthermore, glaucoma is responsible for 16.2 billion dollars of total direct medical costs (7). POAG is disproportionately more prevalent among senior individuals of African descent (>5%) compared to other groups of the population such as Hispanic or Latino (2.7%), Asian (˜2%), and Caucasian (1.5%) (8-10).

Despite the prevalence of POAG, its pathogenesis remains poorly understood. Disruption of the aqueous humor outflow pathway is known to increase the intraocular pressure (IOP), which preferentially and progressively damages the retinal ganglion cells (RGCs) (11). RGCs are involved in the transmission of visual signals from the photoreceptors to the brain (12). They are the first set of neuronal cells to be differentiated in the developing retina, and since RGCs do not regenerate their death results in permanent vision loss. RGC death causes optic nerve atrophy and results in a severe reduction of visual function (2). Elevated IOP is a primary risk factor and the only treatable clinical characteristic of glaucoma (13), however, increased IOP alone is neither sufficient nor necessary to cause RGC death leading to glaucoma (14). According to the Baltimore Eye Survey, approximately 50% of all glaucoma patients have normal-tension glaucoma where the eye pressure is below 22 mm Hg (14). The complexity of glaucoma certainly makes it possible, if not probable, that RGCs become inherently susceptible to this disease process.

Existing treatments for glaucoma target lowering the IOP and include medications such as anti-hypertensive drops, laser trabeculoplasty, and surgical interventions. Surgeries for glaucoma are plagued with complications that result in the need for additional surgery and frequent follow-up. Anti-hypertensive drops have very high rates of non-compliance stemming from the multiple daily drug regimens of eye drops, which can account for 35,000 doses throughout one's lifetime. Importantly, anti-hypertensive drops should ideally be administered to patients before or during early vision loss. But, due to the asymptomatic progression of glaucoma, patients often present with late-stage disease that is irreversible and there are no treatments available once the RGC death has occurred (15). Consequently, there is an unmet need for the development of new strategies for the treatment of glaucoma.

Cell replacement strategies are an innovative and promising alternative to treat glaucoma. One such method involves transferring healthy RGCs into the eyes of afflicted individuals to replace the function of the degenerated and dead ganglion cells. Prior RGC transplantation studies have produced variable outcomes and success (16-25), but thus far have resulted in limited clinical use. The inconsistency in transplantation efficiency may be attributed to the availability and purity of a reliable donor source, differences in graft and host species, and route of delivery.

RGCs are found in the ganglion cell layer of the retina and serve as the projection neurons of the retina, utilizing long axons to effectively connect the eye to the brain. They transmit both image-forming and non-image-forming visual information, processed by retinal cells such as photoreceptors and bipolar cells, to higher visual centers in the lateral geniculate body through the optic nerve. Currently, there are many treatments that slow disease, but no precision treatment exists for glaucoma or RGC degeneration. As mature mammalian RGCs are a terminally differentiated lineage, they do not regenerate after succumbing to disease, consequently leading to irreparable blindness. Understandably, there is a great desire for an application devised to rejuvenate or replace injured RGCs.

In view of the foregoing, it is clear that a need exists for an efficient, reproducible, and safe differentiation protocol for generation of RGCs for use in therapeutic approaches for the treatment of ocular disorders.

### SUMMARY OF THE INVENTION

In accordance with the present invention, a method of preparing retinal ganglion cells by directed differentiation of induced pluripotent stem cells (iPSCs) in a defined chemical medium using 2D-culture system is disclosed. An exemplary method entails culturing iPSC in iPSC initiation medium, transferring iPSCs into retinal progenitor cell (RPC) culture medium for transdifferentiation into RPCs, culturing RPCs in retinal ganglion cell (RGC) culture medium for transdifferentiation into RGCs; thereby providing a population of RGCs suitable for in vitro studies and for transplantation into the eye. In certain embodiments, the iPSCs are cultured in a 37° C., 5% CO₂ and 5% O₂ incubator and the iPSC initiation medium comprises one of: i) 80% HES/20% MEF-CM+20 ng/ml bFGF+4 μM Y27632; ii) 100% HES+20 ng/ml bFGF+4 μM Y27632; or iii) 100% StemMACS™ iPS Brew-Xenofree (XF)+20 ng/ml bFGF+4 μM Y27632.

The RPC culture medium comprises RPC induction media, having 0.1 μM LDN193189, 10 μM SB431542, 2 μM XAV939, 10 mM Nicotinamide, 10 ng/ml IGF1, 1.5 μM CHIR99021 and 10 ng/ml bFGF reagents. The RGC culture medium comprises of RGC induction media, 3 μM DAPT, 250 ng/ml sonic hedgehog (SHH) and 100 ng/ml FGF8 or smoothened agonist (SAG). In certain embodiments, the RGCs produced are isolated. In preferred embodiments, the differentiated RGCs express BRN3A, BRN3B, TUBB3, CD90, MAP2, TUJ1, RBPMS and TUJ1 biomarkers. In some embodiments of the method, the RGC population is obtained after between 30 to 45 days in culture from iPSCs.

The invention also provides a method for the production of retinal progenitor cells (RPCs). An exemplary method comprises culturing iPSCs on 0.1% gelatinized plates containing irradiated MEFs until cells achieve approximately 75% confluence and removing said MEFs and plating remaining iPSCs onto plates containing 1:100 diluted growth factor reduced Matrigel and iPSC:MEF-conditioned medium (80:20)+20 ng/mL of bFGF and 5 ng/ml of stable bFGF until reaching 100% confluence. The iPSC:MEF-conditioned media is then replaced with RPC induction media comprising DMEM/F12 (50:50), 1% Penicillin/Streptomycin, 1% Glut, 1% NEAA, 0.1 mM 2-ME, 2% B27 supplement (w/o vitamin A), 1% N2 supplement, containing effective amounts of a Wnt inhibitor, a TGFβ inhibitor and a BMP inhibitor, 10 mM nicotinamide, and 10 ng/mL IGF1 and cells cultured for 4 days with daily media changes. On day 5, the culture media is replaced with media containing an effective amount of a Wnt inhibitor, a TGFβ inhibitor and a BMP inhibitor, 10 ng/mL IGF1, and 10 ng/mL bFGF on day 5 and cells are cultured for 12 days with daily media changes. The RPC induction media is then replaced with RGC differentiation media comprising 0.1 μM LDN193198, 10 μM SB431542, 2 μM XAV939, 1.5 μM CHIR99021, 10 ng/ml IGF1 and 10 ng/ml bFGF on day 16 of differentiation culturing said cells until Day 23, upon which RGC differentiation occurred, thereby producing a population of RGCs suitable for transplantation into the eye.

In preferred embodiments, the Wnt inhibitor is XAV939, the TGF-β inhibitor is SB431542 and the BMP inhibitor is LDN193189. Stem cells for production of iPSCs, include but are not limited to bone marrow stem cells (BMS), cord blood stem cells, amniotic fluid stem cells, fat stem cells, retinal stem cells (RSCs), keratinocyte stem cells, intraretinal Müller glial cells, embryonic stem cells (ESCs), corneal endothelial cells and somatic cell nuclear transfer cells (SCNTCs). Other Wnt activators useful in the methods of the present invention include for example, Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16b, substance increasing p3-catenin levels; lithium, LiCl, bivalent zinc, BIO (6-bromoindirubin-3′-oxime), SB216763, SB415286, CHIR99021, QS11 hydrate, TWS119, Kenpaullone, alsterpaullone, indirubin-3′-oxime, TDZD-8 and Ro 318220 methanesulfonate salt; Axin inhibitors; APC (adenomatous polyposis coli) inhibitors; norrin and R-spondin 2.

The methods can further comprise the step(s) of determining whether the retinal progenitor cells are differentiated into the mature retinal ganglion cells. In certain embodiments, the step of determining whether the retinal progenitor cells are differentiated into the mature retinal ganglion cells is performed by measuring mRNA or protein expression levels of one or more genes selected from the group consisting of SOX2, RAX, PAX6, SIX6, SIX3, VSX2 and LHX2. In other preferred embodiments, the retinal ganglion cells make up about 60% to about 95% or more of total cells after the RGC differentiation.

In certain approaches, the RGCs comprise an AAV vector for expressing a transgene of interest. In other embodiments, the RGCs are differentiated from iPSCs without genetic manipulation using CRISPR gene editing methodologies.

The invention also provides methods where the iPSCs are differentiated into RGCs from normal and disease patients with ocular pathologies selected from glaucoma, optic neuritis, age-related macular degeneration (AMD) or any type of retinal degeneration, a disorder related to an increase in intraocular pressure (IOP), a disorder related to neuroprotection, or diabetic retinopathy.

A method for the treatment of an ocular disorder is also encompassed by the present invention. An exemplary method comprises administering a therapeutically effective amount of a population of purified RGCs or RPCs described herein either directly, or in a pharmaceutically carrier or along with a neuroprotective agent into a host in need thereof wherein the disorder is selected from glaucoma, age-related macular degeneration (AMD), a disorder related to an increase in intraocular pressure (IOP), a disorder related to neuroprotection, a disorder related to ganglion cell loss, a disorder linked to retinal degeneration or photoreceptor loss or diabetic retinopathy. In preferred embodiments, the host is a human. The cells of the invention may be administered via route selected from intravitreal, intrastromal, intracameral, sub-tenon, sub-retinal, retro-bulbar, peribulbar, suprachoroidal, choroidal, subchoroidal, conjunctival, subconjunctival, epi scleral, posterior juxtascleral, circumcorneal, optic nerve or tear duct injection.

In certain approaches, the RPC or RGC cells are administered through the routes listed above either before optic nerve injury through optic nerve crush or via delivery to the optic nerve. In certain cases, the RGC maturation medium comprises 30 ng/ml to 50 ng/ml BDNF, CNTF or NGF.

In yet another embodiment, a method for the treatment of an ocular disorder, comprising administering a therapeutically effective amount of a population of purified RGCs using immunopanning, flowcytometry and cell sorting with RGC specific surface antibodies, fluorescent tag labeling with RGC specific promoter with or without a lentiviral or adenoviral vector, and in a pharmaceutically carrier to a host in need thereof is provided. Disorders to be treated, include without limitation, glaucoma, age-related macular degeneration (AMD), retinal degeneration, a disorder related to an increase in intraocular pressure (IOP), a disorder linked to optic nerve loss due to trauma or injury, a disorder related to neuroprotection, or diabetic retinopathy. In other embodiments, CD90+ RGCs are immunopurified via MACS sorting with CD90.2 magnetic beads.

### BRIEF DESCRIPTION OF THE DRAWINGS

FIGS. 1A-1D: Differentiation of iPSCs into neural retina and retinal ganglion cells (RGCs). A) Schematic showing iPSCs differentiation in neural retinal differentiation media (DMEM/F12+2% B27+1% N2+10 ng/mL IGF1), including inhibitors of BMP (bone morphogenic protein), WNT and TGF-β for 15 days, bFGF2 was included in media from days 4-14. At 16 days in vitro, cells were treated with RGC induction and maturation media. Differentiation using seven different conditions, L, X, LX, LSB, CHIR, LSB and LSBX are shown. B) Characterization of RPCs using immunocytochemistry with SOX2, RAX and PAX6 along with secondary only control antibodies is shown. C) FACS analysis demonstrated that 96.7% of RPC cells were positive for RAX, and 98.9% of cells were positive for LHX2, when compared to isotype controls. To normalize for flow cytometry cell counts data is represented as % Max. D) Gene expression profiles showing RPC markers in three different iPSC lines, showing a high enrichment. Mean±SEM represented from at least 3 independent experiments per individual.

FIGS. 2A-2E: Efficient generation of iPSC-RGCs require inhibition of TGF-β, Wnt, and BMP signaling. A) Phase contrast images of different stages of iPSC-RGC differentiation at DIV7, DIV16 and DIV35 is shown. B) Differentiation of retinal progenitor cells (RPCs) to retinal ganglion cells (RGCs) using defined inhibition or activation of developmental pathways, C) Percentage of Thy-1 (CD90) positive iPSC-RGCs in our studies in different differentiation conditions, D) Proportions of CD90 positive RGC cells across four differentiation conditions is shown. Each symbol represents an individual experiment. E) Percentage of CD90 positive cells obtained across three control iPSC lines across each differentiation condition. Mean±SEM represented from at least 3 independent experiments per individual.

FIG. 3: Flow cytometry analysis showing percentage of Brn3b positive and RBPMS positive iPSC-RGCs.

FIG. 4: Characterization of iPSC-RGCs after MACS sorting using ICC. The iPSC-RGCs stained positively to RGC markers, Brn3a, TUJ1, MAP2, RBPMS. Positive staining with GFAP showed <5% astrocytes like cells. No cells stained positive with CRALBP.

FIGS. 5A and 5B: Transcriptional Profiling of iPSC-RPCs. Quantitative-RT-PCR profiles showing relative expression of retinal ganglion cell and retinal subtypes (photoreceptors, amacrine, horizontal, and RPE) at Day 23 and Day 35 stages of development from iPSCs to RGCs.

FIGS. 6A-6D: Functional Characteristics of Induced Retinal Ganglion Cells. A) RGCs were transduced with AAV7m8-SNCG-eGFP and immunofluorescence RGC markers (BRN3a, THY1, TUBB3, MAP2, TUJ1, RBPMS). B) Density of RGCs expressing GFP and other axonal processes increases between DIV35 (5B, cell c) and maturation DIV75 (5B, cells a and b). Blue arrows point to cells selected for patch clamp recordings and that fired action potential upon depolarization. C) Action potential firing in response to depolarizing voltage step recorded from cell a and cell b (DIV75) and from cell c (DIV35). D) This trace demonstrates action potential firing in response to the two different depolarizing steps of current recorded from cell a from FIG. 5B. We also observe a spontaneous spike firing at the start of the trace. Same scale bar applies to all images.

FIGS. 7A-7G: FIG. 7A. Sample timeline for protocol for differentiation of iPSC-RGCs using small molecules and proteins. FIG. 7B shows that by day 3, cells continued to expand, with cells reaching more than 100% confluence (Mag-10×). FIG. 7C shows that by day 19, more rosette like cell clusters formed (starting around Day 15), (Mag-10×). FIG. 7D shows that on day 24 before crosshatching, abundant cell clusters with crowded packed cells forming multi layers (light yellow parts). FIG. 7E shows that on day 24, about 3 hours after crosshatching, neuron axon cell bodies start to form (Mag-20×). FIG. 7F shows that on day 27, numerous ganglion cell bodies with extended axons (Mag-10×). FIG. 7G shows Day 37, matured RGC with long axons, RGC cell bodies migrate forming clusters (Mag-10×).

FIGS. 8A-8B: Design and in vitro characterization of AAV2-CAG and AAV7m8 SNCG vectors. FIG. 8A Outline of proviral expression cassettes used in the study. A1 and A2 illustrates comparative vector using AAV2 and CAG promoter with the cDNAs encoding eGFP or SIRT1 respectively. A3 and A4 Illustrates plasmid using the ganglion cell specific promoter, SNCG, driving cDNAs eGFP or SIRT1 respectively. FIG. 8B Fluorescent micrographs of human SIRT1 protein expression in iPSC-RGCs transduced with AAV7m8. SNCG.hSIRT1-3×FLAG. Blue-DAPI nuclear stain, Red-BRN3A nuclear stain, green-3×Flag Tag epitope showing cytoplasmic and nuclear expression from the vector and blue/red/green merged images.

FIGS. 9A-9C: AAV7m8 transduction profile and RGC transduction efficiency following intravitreal delivery. FIG. 9A Representative micrograph of retinal flat mount following intravitreal injection of AAV7m8-SNCG.eGFP. RGCs are labeled with BRN3A (red)(inlet). Representative retinal flat mount used for calculating RGC transduction efficiency with AAV7m8. FIG. 9B Quantification of RGC transduction (n=10, experiments performed in triplicate; retinal whole mounts) comparing AAV2-CAG.SIRT1 vector. FIG. 9C Representative cross-section of mouse retina following intravitreal injection of AAV2-CAG.SIRT1. RGCs are labeled with Brn3a (red) and localized to the ganglion cell layer (GCL). Cells expressing the SIRT1 transgene are labeled green and largely localized to the GCL. Data represented as mean±SEM.

FIGS. 10A-10C: Effect of AAV2 gene transfer on visual acuity and RGC in ONC. OKR recordings demonstrate significantly decreased visual acuity in eyes of ONC mice treated with AAV2-CAG.eGFP (n=10; experiments performed in triplicate). Treatment with AAV2-Mice treated with AAV2-CAG.SIRT1 (n=12; experiments performed in triplicate) show no significant effect on (FIG. 10A) visual acuity or (FIG. 10B) RGC survival by day 7. FIG. 10C Representative RGC counts by nuclear BRN3A staining. Data represented as mean±SEM. *P<0.05, **P<0.01 by 1-way ANOVA with Tukey's HSD post-test.

FIG. 11A-11C: Effect of AAV7m8 gene transfer on visual acuity and RGC in ONC. OKR recordings demonstrate significantly decreased visual acuity in eyes of ONC mice treated with AAV2-eGFP (n=10; experiments performed in triplicate) compared with Sham injured mice (n=10; experiments performed in triplicate ***p=0.002). FIG. 11A) Treatment with AAV7m8-SIRT1 (n=15; experiments performed in triplicate) showed a significant delay in loss of visual function in ONC (n=10; experiments performed in triplicate) compared with Sham injured mice (n=10; experiments performed in triplicate) (*p=0.03) on visual acuity seen in peach area outlined in the graph. FIG. 11B) RGC flat mount counts demonstrate significantly decreased numbers in eyes of ONC mice treated with AAV2-eGFP (n=10; experiments performed in triplicate) compared with Sham injured mice (n=10; experiments performed in triplicate; ***p=0.001). Treatment with AAV7m8-SIRT1 (n=10; experiments performed in triplicate) showed a significant increase in retinal ganglion cell counted per flat month function in ONC (n=7) compared with control ONC mice (n=10; experiments performed in triplicate) (*p=0.03). FIG. 11C) Representative RGC counts by nuclear BRN3A staining Data represented as mean±SEM. *P<0.05, **P<0.01 by 1-way ANOVA with Tukey's HSD post-test.

FIGS. 12A-12C: Functional validation for the association between PPP1R13L and primary open angle glaucoma. FIG. 12A) Differential expression profile of Ppp1r131 transcript in mouse optic nerve head (ONH) with varying stages of intraocular pressure (IOP)-induced glaucoma. Data represent the fold change in Ppp1r131 expression between different stages of D2 mice (glaucoma) and D2 Gpnmb+ (control) samples. * represents q (FDR)<0.05. FIG. 12B) Localization of PPP1R13L protein in the human retina. Shown is the distribution of PPP1R13L by immunohistochemical localization in the retina from normal 68-year-old donor eyes. Overlay of images from DAPI (blue; nuclei) and PPP1R13L (red) in adult human retinal layers are shown on the right. The left represents primary antibody control. Scale bars are shown in each image. ONL, outer nuclear layer; OPL, outer plexiform layer; IPL, inner plexiform layer; GCL, ganglion cell layer. FIG. 12C) Relative expression of PPP1R13L transcript in response to oxidative stress in induced pluripotent stem cell-derived retinal ganglion cells (iPSC-RGCs). Expression of PPP1R13L in iPSC-RGCs is shown under increasing concentrations of H₂O₂ treatment. Plotted are the fold changes in comparison to no H₂O₂. Error bars represent standard error of the mean (SEM). *** denotes p<0.0001, ** denotes p<0.001, and * denotes p<0.01.

FIGS. 13A-13H: FIGS. 13A-13B) Mature iPSC-RGCs were differentiated according to Schematic. FIG. 13C) RPCs characterized using qRT-PCR using gene specific primers and ICC using RPC specified antibodies. FIG. 13D) Detailed description of iPSC-RGC characterization with MACS with CD90 antibody. FIG. 13E) FACS sorting of iPSC-RGCs with different markers. Characterization using ICC markers of RGCs. FIG. 13F) and other neuronal cells FIG. 13G) obtained using our method. FIG. 13H Electrophysiological responses of mature iPSC-RGCs response to light stimuli.

FIGS. 14A-14K: FIG. 14A. The schematic of iPSC-RGCs used for transplantation in a mouse retina. FIG. 14B-14C. Fundus Imaging and SD-OCT of a control mouse which received 1×PBS injection. FIG. 14D-14E. Fundus Imaging and SD-OCT of a mouse which received iPSC-RGC injection. FIG. 14F-14G. Fundus Imaging and follow-up studies for 10 mice (both sexes) to show the reproducibility of integrations post injections. FIG. 14H cryosections of mouse showing integration of eGFP labeled iPSC-RGCs in the retinal ganglion cell layer (left image) and Flatmount retinal imaging (right image) showing transplanted iPSC-RGCs integrated into normal mouse retina. FIG. 14I. Light dependent change in firing when recorded from transplanted iPSC-RGCs (n=6). FIG. 14J-14K. Immunohistochemistry showing staining of transplanted iPSC-RGCs in mouse optic nerve sections two months post-transplantation. iPSC-RGCs are labelled with AAV2.7m8.eGFP and they colocalize with Neurofilament.

FIGS. 15A-15C: Images of hiPSC-RGCs expressing SNCG-eGFP after intravitreal injection. FIG. 15A) Fluorescent images of the hiPSC-RGCs transduced with AAV2.7m8 SNCG-eGFP (4× and 10× magnification). FIGS. 15B, 15C) hiPSC-RGCs transduced with SNCG-eGFP vector were injected into the vitreous space of wild type C57BL/6J mice. Fundus photography of the transplanted hiPSC-RGCs was taken using BAF-cSLO and SD-OCT at 2-, 4-, and 6-weeks post-transplantation.

FIGS. 16A-16J: Images of transplanted hiPSC-RGCs in vivo. FIGS. 16A, 16B, 16C) Fluorescent images of the flattened whole-mount retina of saline-injected left eye and hiPSC-RGCs injected right eye (B=4×, C=10×). FIGS. 16D, 16E, and 16F) Neurite outgrowth of transplanted hiPSC-RGCs taken as two-photon images acquired from live retinal samples. Cryosection images of transplanted hiPSC-RGCs were detected in the GCL and INL layer of the murine retina and co-stained for FIG. 16G-16H) Brn3, FIG. 161) MAP2, and FIG. 16J) Synapsin I.

FIGS. 17A-17H: Immuno-staining of the transplanted hiPSC-RGCs in mouse retina with RGC-specific markers. Flattened whole-mount images of transplanted hiPSC-RGCs were co-stained with RGC-specific markers A) BRN3, B) RBPMS, C) TUJ1, D) MAP2, E) VGLUT2, and F) PSD95. Donor hiPSC-RGCs were also co-stained with G) human nuclear antigen and H) Ku80 to confirm the donor origin of SNCG-eGFP+ cells.* Since the human nuclear antigen antibody was raised in mice, some cross-reactivity with the murine retina was observed. Z-1: Zoom-1, Z-2: Zoom-2, Z-4: Zoom-4, and Z-5: Zoom-5.

FIGS. 18A-18F: Injected hiPSCs integrate into the host retina and generate light responses. Two-photon images at the top row show targeted hiPSC-RGCs (centered) before, during, and after recording its light responses. FIG. 18A) Image shows eGFP fluorescence observed from hiPSC-RGCs in the retinal sample before any attempt to approach it with the recording pipettes. The image is a projection of the Z-stack consisting of 234 optical slices acquired with a 0.5 μm step. FIG. 18B) The red fluorescence from the CF 633 filled pipette and targeted cell is shown. This image comes from the single optical slice acquired at the end of the patch-clamp recording session. The patch-pipette can be seen on the right side of the cell. A slight repositioning of the targeted cell was due to its re-centering for the IR video system used to control pipette manipulations. FIG. 18C) The Z-stack projection (151 optical slices with 0.5 μm step) shows combined eGFP and CF 633 fluorescence after completion of the pipette recording and withdrawal of the patch-pipette. The targeted cell has a bright yellow color. Note that eGFP+ hiPSC-RGC processes observed before the patch-clamp recording (FIG. 18A) can still be readily identified after its completion (FIG. 18C). FIGS. 18D and 18E) Light responses of the targeted cell recorded in the cell-attached configuration (voltage-clamp mode) and whole-cell configuration (voltage-clamp mode) are illustrated. Here upper graphs give the time course of light stimuli and indicate flash intensities, the middle graphs plot current vs time and membrane voltage vs time traces (FIG. 18D and FIG. 18E respectively), and the bottom graphs plot firing rate vs time traces. The current vs time trace from FIG. 18D has been high pass filtered, and the voltage vs time trace from FIG. 18E has not been corrected for the UP, the correction can be done by subtracting 15 mV from the reported values. The firing rate traces were calculated using a 200 ms time bin. The colored light bars indicate light stimulation events. FIG. 18F) Detection of transplanted hiPSC-RGCs in the optic nerve of the mouse and its expression colocalized with the neurofilament marker.

FIGS. 19A-19F: Transplanted hiPSCs increase firing in response to depolarization induced by light or current injection. Two-photon images obtained from the single optical slice acquired at the end of the patch-clamp recording session are shown on the left of the figure. EGFP fluorescence from hiPSCs is illustrated on FIG. 19A, red fluorescence from the CF 633 filled pipette and targeted cell on FIG. 19B, and combined EGFP and CF 633 fluorescence on FIG. 19C. The pipette can be seen on the right side of the cell on FIG. 19B and FIG. 19C. Targeted cell has a bright yellow color on FIG. 19C, the yellowish cell above it was stained with CF 633 during the preceding patching attempt. The scale bar is 50 μm. Traces from FIG. 19D and FIG. 19E show an increase in firing upon light stimulation similar to the corresponding traces from FIG. 18, but with a much lower baseline firing. Firing rates were calculated using a 200 ms time bin. Traces on FIG. 19F illustrate membrane depolarization and action potential firing caused by an injection of depolarizing currents (two current steps of 30 and 40 pA, and the current ramp from 0 to 40 pA). For simplicity, current vs time traces on FIG. 19F (1^{st} and 3^{rd} graphs from the top) show command current, not the actual membrane current measured by the amplifier (the latter will have the same amplitude and time course but will also include electrical noise and spiking activity). The membrane voltage from FIG. 19E and FIG. 19F wasn't corrected for UP, correction can be done by subtracting 15 mV from the reported values.

FIGS. 20A-20E: Example of hiPSC firing triggered by large and mostly spontaneous depolarizations. Two-photon images (single optical slice) on FIGS. 20A, 20B, and 20C show EGFP, CF 633, and combined EGFP+CF 633 fluorescence respectively at the end of the patch-clamp recording. The scale bar is 50 μm. Graphs from FIG. 20D show firing (bottom trace, firing rate calculated using 200 ms time bin) triggered by two depolarizations, one aligned with the flash and a spontaneous one (middle trace). The trace at the top shows the time course of the stimulation, colored bars indicate light stimulation. Traces on FIG. 20E show membrane depolarization and action potential firing in response to current injections (two steps of 20 and 30 pA and a ramp from 0 to 40 pA). To correct for the UP 15 mV should be subtracted from the reported membrane voltages. All traces were recorded in the current-clamp mode.

FIGS. 21A-21C: Larger membrane depolarizations caused by repeated stimuli can lead to smaller increases in the firing rate. All traces were recorded in the current-clamp mode from the same cell as that presented in FIG. 18. FIGS. 21A and 21B show depolarization and a related increase in the firing rate caused by light exposures. The flash from FIG. 21B was delivered 30 s after that from FIG. 21A. Traces on FIG. 21C were recorded following repeated stimulation with two flashes like that and including that illustrated on FIG. 21B, and two 2× brighter flashes. The traces at the top of each panel illustrate the time course and intensity of the stimulation (FIG. 21C trace shows command current, not the actual membrane current recorded by the amplifier). Stimulation events are also indicated by the colored (light stimulation) and gray (current injection) bars. The firing rates were calculated using a 200 milliseconds time bin. For each stimulation time bins were aligned with stimulation onset (stimulation onset is between two-time bins). The membrane voltages were not corrected for the UP, correction can be done by subtracting 15 mV from the reported values.

FIGS. 22A-22B: Fundus image of a murine eye was taken following Pronase E injection in BAF and IR mode in the cSLO. 0.0001% of Pronase E was intravitreally injected into the vitreous cavity of C57BL/6 mice to degrade the ILM/NFL layer of the retina. The use of Pronase E FIG. 22A) caused cataract formation and FIG. 22B) induced inflammation as observed in the fundus image obtained in the BAF and IR modes of the cSLO.

FIG. 23: Sample Transplantation of iPSC-RGCs and RPC in human eyes.

### DETAILED DESCRIPTION

Glaucoma is a group of progressive optic neuropathies that share common biological and clinical characteristics including irreversible changes to the optic nerve and visual field loss caused by death of retinal ganglion cells (RGCs). The loss of RGCs manifests as characteristic cupping or optic nerve degeneration, resulting in visual field loss in patients with Glaucoma. Published studies on in vitro RGC differentiation from stem cells utilized classical RGC signaling pathways mimicking retinal development in vivo. Although many strategies allowed for the generation of RGCs, increased variability between experiments and lower yield hampered the cross comparison between individual lines and between experiments. To address this critical need, we developed a reproducible, chemically defined in vitro methodology for generating retinal progenitor cell (RPC) populations from iPSCs, that are efficiently directed towards RGC lineage. Using this method, we reproducibly differentiated iPSCs into RGCs with greater than 80% purity, without any genetic modifications. We used small molecules and peptide modulators to inhibit BMP, Transforming growth factor beta (TGF-β or SMAD), and canonical Wnt pathways that reduced variability between iPSC lines and yielded functional and mature iPSC-RGCs. Using CD90.2 antibody and Magnetic Activated Cell Sorter (MACS) technique, we successfully purified Thy-1 positive RGCs with nearly 95% purity.

The RGCs developed in accordance with the invention can be used to express transgenes of interest to assess their effects on RGC viability and function. For example, SIRT1 prevents retinal ganglion cell (RGC) loss in models of optic neuropathy following pharmacologic activation or genetic overexpression. The exact mechanism of loss is not known, although prior evidence suggests this due to oxidative stress to either neighboring cells or RGC specifically. In Example 2, the neuroprotective potential of RGC-selective SIRT1 gene therapy in the optic nerve crush (ONC) model was assessed. We hypothesized that AAV-mediated overexpression of SIRT1 in RGCs reduces RGC loss, thereby preserving visual function. Cohorts of C57Bl/6J mice received intravitreal injection of experimental or control AAVs using either a ganglion cell promoter or a constitutive promoter and ONC was performed. Visual function was examined by optokinetic response (OKR) for 7 days following ONC. Retina and optic nerves were harvested to investigate RGC survival by immunolabeling. The AAV7m8-SNCG.SIRT1 vector showed 44% transduction efficiency for RGCs compared with 25% (P>0.05) by AAV2-CAG.SIRT1, and AAV7m8-SNCG.SIRT1 drives expression selectively in RGCs in vivo. Animal modeling of ONC demonstrated reduced visual acuity compared to controls. Intravitreal delivery of AAV7m8-SNCG.SIRT1 mediated significant preservation of the OKR and RGC survival compared to AAV7m8-SNCG.eGFP controls, an effect not seen with the AAV2 vector. RGC-selective expression of SIRT1 offers a targeted therapy in an animal model with significant ganglion cell loss. Over-expression of SIRT1 through AAV-mediated gene transduction suggests a RGC selective component of neuro-protection using the ONC model. This study expands our understanding of SIRT1 mediated neuroprotection in the context of compressive or traumatic optic neuropathy, making it a strong therapeutic candidate for testing in all optic neuropathies. Notably, application of resveratrol has been observed to preserve visual function and RGC survival by activating the SIRT1 pathway.

Leveraging 10,900 whole exomes linked to EHR data in the Penn Medicine Biobank PMBB) for discovery, we addressed the association of the cumulative effect of rare predicted loss-of-function (pLOF) variants per gene on an exome-wide scale in iPSC-RGCs. After discovering several significant gene-disease associations (p<10-6), we robustly replicated and tested these gene-disease associations in RGCs. This analysis revealed that variants in the PPPIR13L gene are associated with primary open angle glaucoma and the RGCs can be used as ideal in vitro models to study RGC pathobiology, evaluate stress conditions using chemicals and reagents to mimic Glaucoma or optic neuritis.

We further transplanted mature human iPSC-derived retinal ganglion cells (hiPSCs-RGCs) intravitreally in wild type C57BL/6J mice. Our protocol for generating hiPSC-RGCs represents one of the most versatile, robust, and highly reproducible methods for the large-scale production of pure RGC populations without any gene modification (26). Following intravitreal injections, eGFP+hiPSC-RGCs were detected in 15 of the 16 wild type mice, with an average successful transplantation rate of about 94%. Overall, an average of 672 donor hiPSC-RGCs were visible per explanted host retina. The transplanted RGCs integrated within the ganglion cell layer of host retinas and survived at least 5-months post-transplantation. Furthermore, the transplanted hiPSC-RGCs were functional and showed electrophysiologic profiles similar to native mouse RGCs. The data disclosed herein indicate that hiPSC-RGCs are a necessary source of donor cells that may aid in the development of patient-tailored cell therapies designed to improve visual function in glaucoma.

Disclosed herein are methods for generating RPCs and RGCs from iPSCs and methods of using said RGCs for treatment of ocular disorders and in screening assays to identify new biomarkers of disease and efficacious agents for the treatment of the same.

### Definitions

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific, and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

A “clonal cell population” refers to a group of identical cells that are derived from the same cell.

“Multipotent” implies that a cell is capable, through its progeny, of giving rise to several different cell types found in the adult animal.

“Pluripotent” implies that a cell is capable, through its progeny, of giving rise to all the cell types which comprise the adult animal including the germ cells. Both embryonic stem and embryonic germ cells are pluripotent cells under this definition.

The term “cell line” as used herein can refer to cultured cells that can be passaged at least one time without terminating. The invention relates to cell lines that can be passaged at least 1, 2, 5, 10, 15, 20, 30, 40, 50, 60, 80, 100, and 200 times. Cell passaging is defined hereafter.

The term “suspension” as used herein can refer to cell culture conditions in which cells are not attached to a solid support. Cells proliferating in suspension can be stirred while proliferating using apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “reprogramming” or “reprogrammed” as used herein can refer to materials and methods that can convert a cell into another cell having at least one differing characteristic. Also, such materials and methods may reprogram or convert a cell into another cell type that is not typically expressed during the life cycle of the former cell. For example, (1) a non-totipotent cell can be reprogrammed into a totipotent cell or (2) a precursor cell can be reprogrammed into a totipotent cell.

The term “differentiated cell” as used herein can refer to a precursor cell that has developed from an unspecialized phenotype to a specialized phenotype. For example, embryonic cells can differentiate into an epithelial cell lining the intestine. Materials and methods of the invention can reprogram differentiated cells into totipotent cells. Differentiated cells can be isolated from a fetus or a live born animal, for example.

The term “undifferentiated cell” as used herein can refer to a precursor cell that has an unspecialized phenotype and is capable of differentiating. An example of an undifferentiated cell is a stem cell.

The term “differentiated cell” means any cell that does not have stem cell capacity, where stem cell capacity is the ability to divide in a manner that renews a baseline state of relative undifferentiation (stem cell phenotype) will simultaneously producing cells of different and developmentally more restricted state of differentiation (i.e. differentiated non-stem cells). In practice, the terms differentiated and undifferentiated always require a developmental reference, whether explicit or implicit. Tissue stem cells are undifferentiated relative to their differentiated, non-stem progeny cells. However, relative to iPSCs, embryonic stem cells, and embryonic precursor cells, tissue stem cells are more differentiated. It should be noted that placing many primary cells in culture can lead to some loss of fully differentiated characteristics. Thus, simply culturing such cells are included in the term differentiated cells and do not render these cells tissue stem cells or pluripotent cells. The transition of a differentiated cell to pluripotency requires a reprogramming stimulus beyond the factors that lead to partial loss of differentiated character in culture. Reprogrammed cells also have the characteristic of the capacity of extended passaging without loss of proliferative potential, relative to their primary cell parents, which generally have capacity for only a limited number of divisions in culture.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germ line cells. In mammals, germ line cells (also known as “gametes”) are the spermatozoa and ova, which fuse during fertilization to produce a cell called a zygote, from which the entire mammalian embryo develops. Every other cell type in the mammalian body (aside from the sperm and ova), is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments, the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when a differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., Meneghel-Rozzo et al., 2004, Cell Tissue Res, 316:295-303, which is incorporated herein in its entirety by reference.

As used herein, the term “adult cell” refers to a cell found throughout the body after embryonic development.

As used herein, the terms “iPSC”, “iPS cell” and “induced pluripotent stem cell” are used interchangeably and refers to a pluripotent cell technically derived (e.g., induced by complete or partial reversal) from a differentiated cell (e.g. a non-pluripotent cell), typically an adult differentiated cell.

The term “progenitor cell” is used herein to refer to cells that have a cellular phenotype that is at an earlier step along a developmental pathway or progression than is a later differentiated cell relative to a cell to which it can give rise by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells are distinct from tissue stem cells in that they lack asymmetric self-renewal. In the absence of their own producer stem cell, progenitor cells' populations are rapidly exhausted because of their inability to simultaneously preserve their own initial degree of differentiation.

The phrase “Retinal Progenitor Cell” or “RPC” refers to multipotent progenitor cells that can give rise to all the various cell types of the retina. The cells retinal ganglion cells, amacrine cells, bipolar cells, horizontal cells, rod photoreceptors, cone photoreceptors, and Müller glia cells. The differentiation of retinal precursor cells into the mature cell types found in the retina is coordinated in time and space by factors within the cell as well as factors in the environment of the cell.

In the context of cell ontogeny, the term “differentiate”, or “differentiating” is a relative term meaning a “differentiated cell” which has progressed further down the developmental pathway than its precursor cell. Thus, in some embodiments, a reprogrammed cell as this term is defined herein, can differentiate to lineage-restricted precursor cells (such as a mesodermal stem cell or a endodermal stem cell), which in turn can differentiate into other types of precursor cells further down the pathway (such as a tissue specific precursor, for example, a retinal precursor, an eye-specific cell type precursor, a cardiomyocyte precursor, or a pancreatic precursor), and then to an end-stage differentiated cell, which plays a characteristic role in a certain tissue type, and may or may not retain the capacity to proliferate further.

The term “exogenous” refers to a substance present in a cell that was introduced from outside the cell by either a natural process or via genetic recombination. The terms “exogenous” when used herein refers to a nucleic acid (e.g., a nucleic acid encoding a reprogramming transcription factor) or a protein (e.g., a transcription factor polypeptide) that has been introduced by a process involving the hand of man into a biological system such as a cell or organism in which it is not normally found or in which it is found (17) in lower amounts. A substance (e.g. a nucleic acid or a protein) will be considered exogenous if it is introduced into a cell or an ancestor of the cell that inherits the substance. In contrast, the term “endogenous” refers to a substance that is produced within the cell by natural processes.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “contacting” or “contact” as used herein as in connection with contacting a differentiated cell (e.g. tissue stem cell) with a compound as disclosed herein (e.g., an expression vector), includes subjecting the cell to a culture media, which comprises the compound. Where the differentiated cell is in vivo, contacting the differentiated cell with a compound includes administering the compound in a composition to a subject via an appropriate administration route such that the compound contacts the differentiated cell in vivo.

As used herein, the term “expanding” refers to increasing the number of like cells through symmetrical cell division (mitosis). The term “proliferating” and “expanding” are used interchangeably.

The phrase “optic neuropathy” refers to optic nerve abnormalities or damage. This damage could be from blocked blood flow, certain medical conditions or toxic exposure. A “progressive optic neuropathy” refers to an optic neuropathy that causes progressive vision loss. “Glaucoma” is a group of progressive optic neuropathies that share common biological and clinical characteristics including irreversible changes to the optic nerve and visual field loss caused by the death of retinal ganglion cells (RGCs). The loss of RGCs manifests as characteristic cupping or optic nerve degeneration, resulting in visual field loss in patients.

The phrase “retinal ganglion cells” or “RGCs” refers to a type of neuron located near the inner surface (the ganglion cell layer) of the retina of the eye. Retinal ganglion cells collectively transmit image-forming and non-image forming visual information from the retina to several regions in the thalamus, hypothalamus, and mesencephalon, or midbrain.

With reference to nucleic acids of the invention, the term “isolated nucleic acid” is sometimes used. This term, when applied to DNA, refers to a DNA molecule that is separated from sequences with which it is immediately contiguous (in the 5′ and 3′ direction) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

The term “promoter region or expression control sequence” refers to the transcriptional regulatory regions of a gene, which may be found at the 5′ or 3′ side of the coding region, or within the coding region, or within introns. Such sequences regulate expression of a polypeptide coded for by a polynucleotide to which it is functionally (“operably”) linked. Expression can be regulated at the level of the mRNA or polypeptide. Thus, the term expression control sequence includes mRNA-related elements and protein-related elements. Such elements include promoters, domains within promoters, upstream elements, enhancers, elements that confer tissue or cell specificity, response elements, ribosome binding sequences, transcriptional terminators, etc.

Those skilled in the art will recognize that a nucleic acid vector can contain nucleic acid elements other than the promoter element and the nucleic acid molecule of interest. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

In some embodiments, the expression control sequence comprises a tissue- or organ-specific promoter. Many such expression control sequences will be evident to the skilled worker.

The term “vector” refers to a small carrier DNA molecule into which a DNA sequence can be inserted for introduction into a host cell where it will be replicated. An “expression vector” is a specialized vector that contains a gene or nucleic acid sequence with the necessary regulatory regions needed for expression in a host cell.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, a viral vector, a naked plasmid and the like.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

As used herein, the term “treatment” refers to the application or administration of a therapeutic agent to a patient, or application or administration of a drug to an isolated tissue or cell line from a patient, who has a medical condition, e.g., a disease or disorder, a symptom of disease, or a predisposition toward a disease, with the purpose to cure, heal, alleviate, relieve, alter, remedy, ameliorate, improve, or affect the disease, the symptoms of disease, or the predisposition toward disease. In an alternative embodiment, tissues or cells or cell lines from a normal donor may also be “treated”.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

A “pharmaceutical composition” comprises a pharmacologically effective amount of a therapeutic agent, optionally other drug(s), and a pharmaceutically acceptable carrier. As used herein, “pharmacologically effective amount,” “therapeutically effective amount” or simply “effective amount” refers to that amount of an agent effective to produce a commercially viable pharmacological, therapeutic, preventive or other commercial result.

“Pharmaceutically acceptable carrier” refers to a carrier or diluent for administration of a therapeutic agent. Pharmaceutically acceptable carriers for therapeutic use are well known in the pharmaceutical art, and are described, for example, in Remington's Pharmaceutical Sciences, A R Gennaro (editor), 18.sup.th edition, 1990, Mack Publishing, which is hereby incorporated by reference herein. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756. The single or double stranded oligos of the present invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, prodrugs and pharmaceutically acceptable salts of the compounds of the invention, pharmaceutically acceptable salts of such prodrugs, and other bioequivalents are also encompassed by the present invention. In addition, single stranded oligos may be formulated for oral delivery (Tillman et al., J Pharm Sci 97: 225, 2008; Raoof et al., J Pharm Sci 93: 1431, 2004; Raoof et al., Eur J Pharm Sci 17: 131, 2002; U.S. Pat. No. 6,747,014; U.S. 2003/0040497; U.S. 2003/0083286; U.S. 2003/0124196; U.S. 2003/0176379; U.S. 2004/0229831; US 2005/0196443; U.S. 2007/0004668; U.S. 2007/0249551; WO O_{2/092616}; WO 03/017940; WO 03/018134; WO 99/60012). Such formulations may incorporate one or more permeability enhancers such as sodium caprate that may be incorporated into an enteric-coated dosage form with the oligo.

The term “inhibitor” refers to compounds and/or molecules that reduce the expression, amount, or activity of another biological compound. The phrase “bone morphogenic protein inhibitor” or “BMP inhibitor” refers to an agent that inhibits BMP activity and/or BMP nucleic acid expression.

The phrase “TGF-β inhibitor” or “transforming growth factor beta inhibitor” refers to an agent that inhibits TGF-β activity and/or TGF-β nucleic acid expression.

The phrase “Wnt signaling pathway” refers to an evolutionarily conserved pathway that regulates crucial aspects of cell fate determination, cell migration, cell polarity, neural patterning and organogenesis during embryonic development. The Wnts are secreted glycoproteins that act as short-range ligands to activate receptor-mediated signaling pathways and comprise a large family of proteins in humans. To date major signaling branches downstream of the Fz receptor have been identified including a canonical or Wnt/β-catenin dependent pathway and the non-canonical or β-catenin-independent pathway. The phrase “canonical Wnt pathway” or “canonical Wnt signaling pathway” refers to Wnt signaling that inhibits the degradation of β-catenin, which can regulate transcription of a number of genes. Canonical WNTs control the β-catenin dynamics as the cytoplasmic level of β-catenin is tightly regulated via phosphorylation by the ‘destruction complex’, consisting of glycogen synthase kinase 3β (GSK3β), casein kinase 1α (CK1α), the scaffold protein AXIN, and the tumor suppressor adenomatous polyposis coli (APC). An “inhibitor of the canonical WNT pathway” refers to any agent or compound that inhibits this pathway.

The phrase “optokinetic response” refers to a combination of a slow and fast phase eye movements. It is observed seen when a mouse/individual tracks (pursuit movement) a moving object with their eyes, which then moves out of the field of vision at which point their eye moves back to the position it was in (saccade movement) when it first saw the object. The reflex/response is monitored and tracked as a measure to assess visual function.

The following examples are provided to illustrate certain embodiments of the invention. They are not intended to limit the invention in any way.

### Example 1

### Methods for Reproducible Differentiation of iPSCs into RPCs and RGCs

We detail a chemically defined, in vitro technique for cultivating RPC populations from iPSCs that are then directed toward the RGC lineage. True to their in vivo development, we directed control lines of iPSCs to differentiate in a stepwise manner using a temporal induction method centered on small molecule and peptide modulator treatment to inhibit BMP and TGF-β (SMAD), and canonical Wnt, yielding a robust population of iPSC-RGCs after Day 36. Analysis at the molecular and physiologic levels using flow cytometry, immunolabeling, gene expression, and electrophysiology yielded results in accordance with the RGC lineage and allowed specific subtype identification.

The following materials and methods are provided to facilitate the practice of the Example 1.

Human iPSC Culture

Undifferentiated iPS cells, Control 1 (CHOPWT8), Control 2 (CHOPWT9), and Control 3 (CHOPWT10) were derived and characterized as previously published showing a complete analysis of iPSC characteristics.³⁴⁻³⁶ All human sample collection protocols were approved by the University of Pennsylvania and Children's Hospital of Philadelphia Human Subjects Research Institutional Review Board following the Declaration of Helsinki. All methods were performed in accordance with the relevant research guidelines and regulations of University of Pennsylvania. Written informed consent was obtained from all human cell donors. The iPSC cells were maintained in iPSC medium (Dulbecco's modified essential medium/Ham's F12 nutrient media; DMEM/F12 (50:50; Corning)) containing 1% Glutamax, 1% penicillin/streptomycin (PS), 15% Knockout serum replacement (KSR), 1% non-essential amino acids (NEAA), 0.1 mM β-Mercaptoethanol (2-ME) (Life Technologies, CA), and 5 ng/mL of basic fibroblast growth factor (bFGF; R&D Systems) on 0.1% gelatin coated dishes with irradiated mouse embryonic fibroblast (iMEFs).

### Retinal Progenitor Cell Generation and Conditions

iPSCs were cultured on 0.1% gelatinized plates containing iMEFs in 37° C. 5% O₂ and 5% CO₂ conditions. Cells were maintained until approximately 75% confluence, then feeder cells were depleted and approximately 1.5×10⁶ iPSCs were seeded in one well of 6-well tissue culture dish (Corning) coated with 1:100 diluted growth factor reduced Matrigel. iPSCs were maintained in iPSC: MEF-conditioned medium (80:20)+20 ng/mL of bFGF and 5 ng/ml of stable bFGF. The MEF-conditioned media was prepared by plating iMEFs onto 0.1% gelatin at a density of 20,000 cells/cm² in iPSC media. Two days post-plating, media was collected, filtered, and either used directly or cryopreserved for later use. iPSCs were maintained in 37° C. at 5% O₂ and 5% CO₂ until reaching 100% confluence then transferred to 37° C., 5% CO₂ overnight prior to induction. On day 0, iPSC: MEF-conditioned media was changed to RPC induction media: DMEM/F12 (50:50), 1% P/S, 1% Glut, 1% NEAA, 0.1 mm 2-ME, 2% B27 supplement (w/o vitamin A), 1% N2 supplement, containing 2 μM XAV939 (X) (Wnt inhibitor; SelleckChem), 10 μM SB431542 (SB) (TGF-β inhibitor; SelleckChem), 100 nM LDN193189 (L) (BMP inhibitor; Stemgent), 10 mM nicotinamide (Sigma-Aldrich), and 10 ng/mL IGF1 (R&D Systems). Cultures were fed daily for 4 days. On day 4, culture media was exchanged with RPC induction media containing: 2 μM XAV939, 10 μM SB431542, 100 nM LDN193189, 10 ng/mL IGF1, and 10 ng/mL bFGF. Media was changed daily for 12 days. On day 16 of differentiation, seven conditions—1) L, 2) X, 3) 1.5 μM CHIR99021 (CHIR) (Wnt agonist; SelleckChem), 4) LX, 5) LSB, 6) LSB-CHIR and 7) LSBX were evaluated for RPC and RGC markers on day 23. Cultures from the following four conditions—1) LSBX, 2) LSB, 3) LX, and 4) 1.5 μM CHIR99021 (CHIR) (Wnt agonist; SelleckChem) were further evaluated at 35. An additional five conditions—1) L only, 2) X only, 3) LSB, 4) LSBX, and 5) LSB-CHIR were evaluated for RPC markers on day 23 to evaluate for transcript expression differences across different conditions in RPCs. Cultures were treated for 6 days before transcript analysis.

### Retinal Ganglion Cell (RGC) Differentiation.

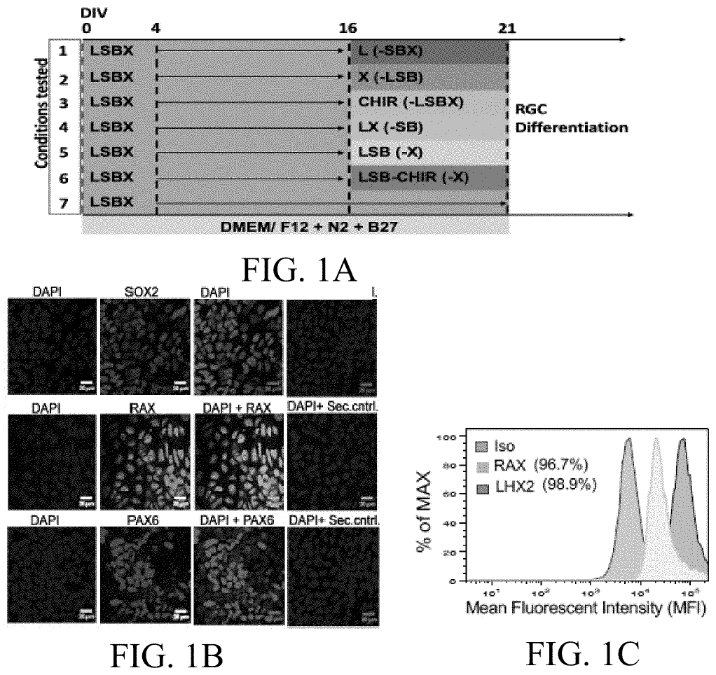

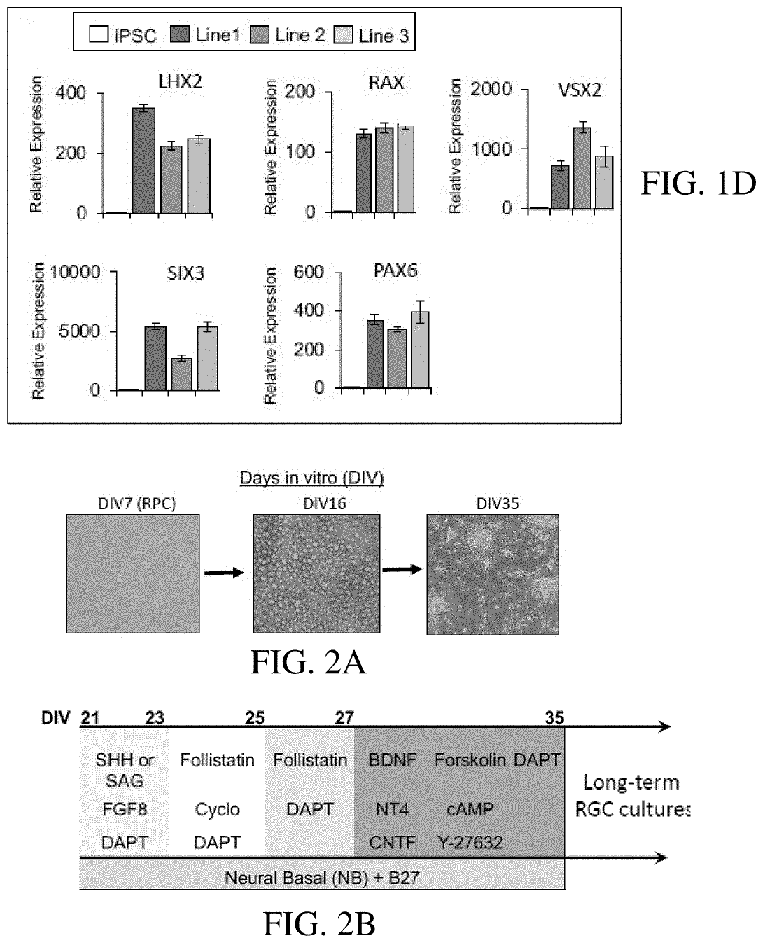

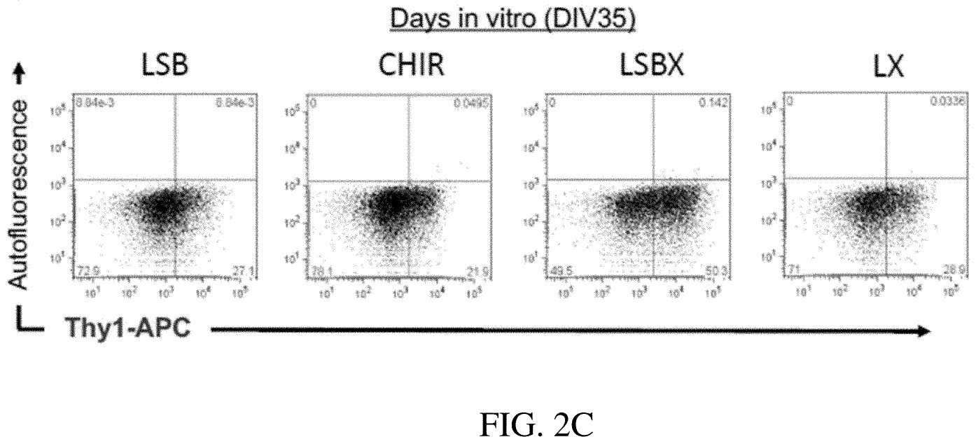

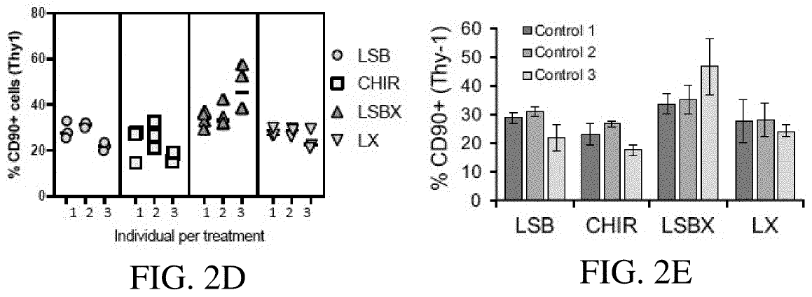

Prior to RGC differentiation, medium was changed daily using RGC induction media containing SHH (sonic hedgehog) (250 ng/mL; R&D systems) or SAG (100 nM; Tocris), SHH was replaced with SAG after establishing equal competence, FGF8 (100 ng/mL, R&D systems) for two days. For RGC differentiation (see FIGS. 1D and 2B for kinetics), on day 24, cells were manually crossed into small clusters with Leibovitz's medium containing 34 uM Glucose using the crosshatching technique as previously described (Espuny-Camacho et al., 2013), then replated at a density of 1.0×10⁵ cells/well of 6-well plate coated with 1:100 diluted growth factor reduced Matrigel in RGCs induction media containing: Follistatin 300 (100 ng/ml), cyclopamine (0.5 uM, Tocris), DAPT (3 uM, Tocris), and 4.2 μM Rock inhibitor (Y27632). Media was changed 24 h post-plating with RGC induction media containing: Follistatin 300 (100 ng/ml) and DAPT (3 uM), daily for two days. From day 27, media was changed to RGC induction media containing: DAPT (3 uM, Tocris), 10 μM Rock inhibitor (Y27632), forskolin (5 uM; SelleckChem), cAMP (400 uM; Sigma-Aldrich), BDNF (40 ng/mL; R&D systems), NT4 (5 ng/mL; R&D systems), and CNTF (10 ng/mL; R&D systems). Media was changed every 2-3 days until day 36. After maturation (D36), medium was exchanged every 3-4 days with RGC induction media containing: DAPT (3 uM, Tocris), 10 μM Rock inhibitor (Y27632). Detailed step-wise methodology of iPSC-RGC generation, reagents and concentrations used in our study are provided below. In certain embodiments, activators of the SIRT1 pathway, such as resveratrol, may be included in the differentiation media during RGC differentiation.

Protocol for Differentiation of iPSC-RGCs Using Small Molecules and Proteins

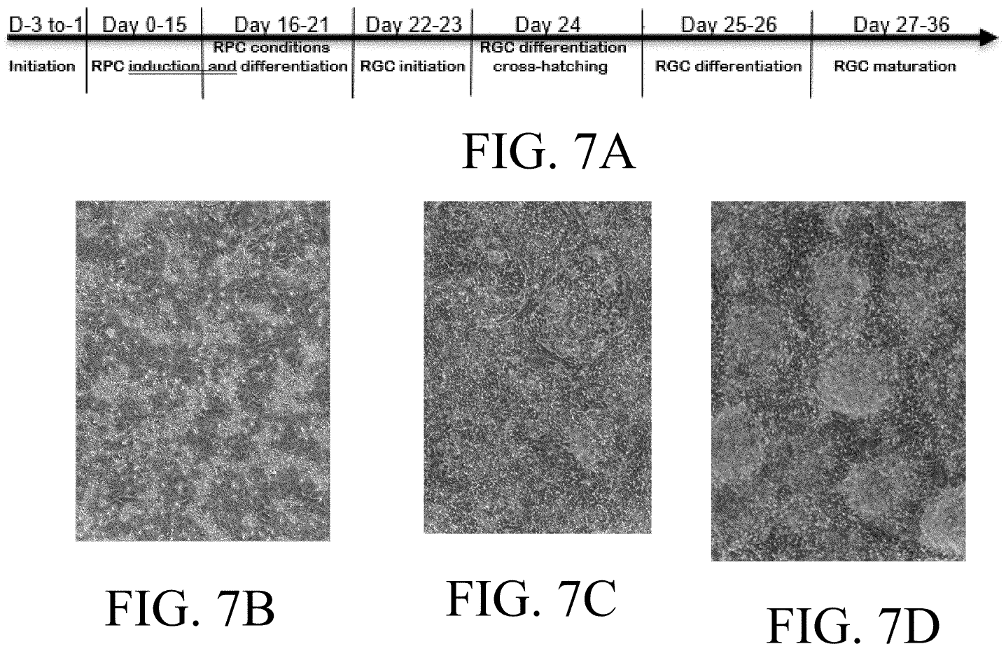

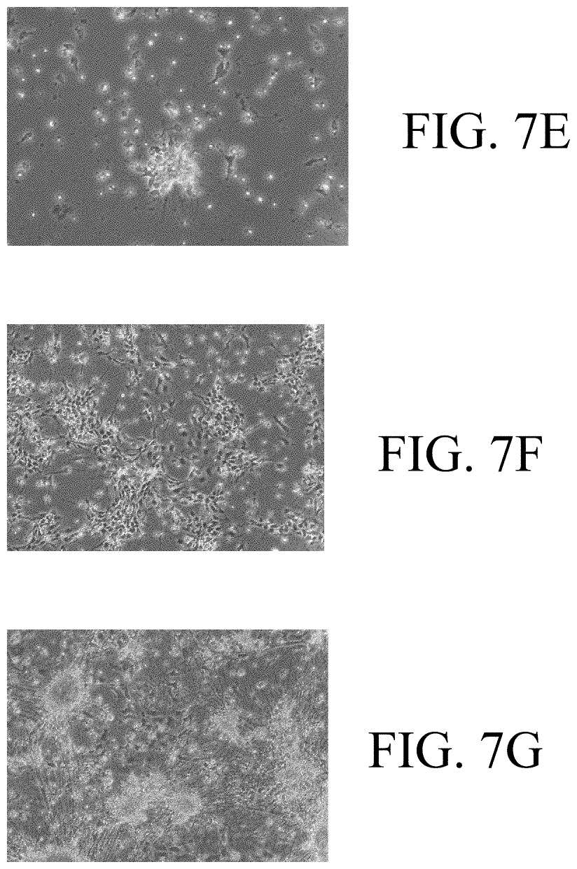

A timeline showing the differentiation of iPSC-RGCs is shown in FIG. 7A.

iPSC Initiation: Day-3 to Day-1

Day1: MEFs cell depletion: When iPSC cells reach 80% confluent, 1 ml of TrypLE solution was applied and the cells incubated at 37° C. for 4-5 minutes. The TrypLE medium was removed after incubation and iPSCs replated into a 100 mm dish precoated with matrigel, in medium containing: 80% HES/20% MEF-CM+20 ng/ml bFGF+4 uM Y27632. The plate was incubated in a 37° C., 5% CO₂ and 5% O₂ incubator. In an alternate embodiment, when iPSC cells reach 60% confluence, the iPSC cells are plated on 100 mm dish precoated with Matrigel (without MEFs) and in medium containing either:

i) 80% HES/20% MEF-CM+20 ng/ml bFGF+4 uM Y27632. Cells incubated in 37° C., 5% CO₂ and 5% O₂ incubator.

ii) 100% HES+20 ng/ml bFGF+4 uM Y27632. Cells incubated in 37° C., 5% CO2 and 5% O₂ incubator.

iii) 100% STEMACS iPS Brew XF (Miltenyi Biotec)+20 ng/ml bFGF+4 uM Y27632. Cells incubated in 37° C., 5% CO₂ and 5% O₂ incubator.

In certain embodiments Vitronectin (without MEFs) may replace Matrigel as a matrix for iPSC growth.

Day2: Second MEFs cell depletion step: After iPSC cells reach 80% or more confluency, 2 ml of TrypLE solution was added to 100 mm dish and incubated at 37° C. for 4-5 minutes. After TrypLE treatment, iPSC cells were replated into one well of a 6 well plate, precoated with Matrigel (seeding density of 1.5×10⁶ cells per well), in medium containing: 80% HES/20% MEF-CM+20 ng/ml bFGF+5 ng/ml stable bFGF+4 uM Y27632 and incubated in 37° C., 5% CO₂ and 5% O₂ incubator.

In an alternative embodiment, iPSCs at 80% confluence are cultured on 100 mm dishes precoated with Matrigel/Vitronectin in the absence of MEFs. Media is then replaced with any of the above three conditions without passaging. The Day1 culture will yield highly pure iPSC-RGCs following the differentiation protocol and avoid using MEF cells during the process of differentiation.

Day3: The spent medium is replaced with a fresh medium without Y27632. (selected from any of i), ii), or iii) listed above). The plate is moved from the 5% O₂ incubator to the regular O₂ incubator and maintained in 37° C., 5% CO₂ incubator (the night before) to continue with RPC induction and differentiation.

### RPC Induction and Differentiation: Day 0-21

Day 0-Day 3: medium is changed daily with RPC induction media containing: 0.1 μM LDN, 10 μM SB, 2 μM XAV, 10 mM NIC, 10 ng/ml IGF1. All small molecule and proteins added into each day's medium are freshly aliquoted (just before medium change). (See FIG. 7B).

TABLE 1
RPC induction Media

| | | Final |
|---|---|---|
| | Reagent | Concentration |
| 1 | Regular DMEM/F12 | |
| 2 | 1 x Penn/Strep (P/S; 100X) | 1% |
| 3 | 1 x Glutamine MAX (100X) | 1% |
| 4 | NEAA (MEM Non Essential Amino acid) | 1% |
| 5 | 2-Mercaptoethanol (55 mM stock) | 0.1 mM |
| 6 | N2 supplement (1%) | 1% |
| 7 | B27 Supplement without Vitamin A (2%) | 2% |

Day 4-Day 21: Medium is changed daily with RPC induction media containing: 0.1 μM LDN, 10 μM SB, 2 μM XAV, 1.5 μM. CHIR, 10 ng/ml IGF1, and 10 ng/ml bFGF. All small molecules and proteins are into each day's medium after freshly aliquoting just before medium change. (FIG. 7C)

### RGC Initiation and Differentiation: Day 22-36

Day 22-Day 23: Medium is changed daily with RGC induction media containing: 3 μM DAPT, 250 ng/ml SHH, and 100 ng/ml FGF8. (FIG. 7D)

TABLE 2
RGC Induction Media

| | | |
|---|---|---|
| 1 | Regular DMEM/F12 | 50% |
| 2 | Neuraobasal media | 50% |
| 3 | 1 x Penn/Strep (P/S; 100X) | 1% |
| 4 | 1 x Glutamine MAX (100X) | 1% |
| 7 | B27 Supplement without Vitamin A (2%) | 2% |

On Day 24:
1. 30 ml of Leibovitz's (LM) medium L15+1 M Glucose is added into 50 ml tube stored in an ice bucket.
2. The 6 well cell plate is removed from the incubator, and the wells washed once with cold LM+Gluc. (LMG) media (2 mL/well).
3. 2 mL of LMG media is added to each well of the 6-well dish and the cells firmly scraped in horizontal and vertical lines across the bottom surface of the well using a p1000 tip with the pipette, to dislodge cells into small clusters. Rinse plate surface well with a pipette to ensure all cells are being removed (>90%). This can be achieved by drawing medium up and down several times, each time to rinse off cells from different bottom area. Pipetting should be done until any cell clusters are broken down. Cells are then checked under a microscope, if cell clusters remain large, pipette them again vigorously to obtain single cells.
4. Cells are removed with LM+Gluc from above into 20 mL of LMG media in a 50 ml tube, and centrifuged at 3000 rpm for 3 min.
5. The supernatant is removed and cell pellets resuspended in 8 ml Day24 medium+4 uM Y27632), and the cells pipetted up and down about 4-6 times to break up clumps or clusters. Day24 medium+Y27632 is then added for plating. Cell seeding density should be about 1×10⁵/well of 6 well plates. (FIG. 7E)

Day 25-26: Change medium daily with RGC induction media containing: 3 μM DAPT and 100 ng/ml Follistatin

Day 27-36: Change medium every 2-3 days with RGC induction media containing: 3 μM DAPT, 10 μM Y27632, 400 μM cAMP, 5 μM Forskolin, 40 ng/ml BDNF, 5 ng/ml NT4 and 10 ng/ml CNTF. (FIG. 7F). In certain embodiments, the RGC induction media may contain concentrations of BDNF may be selected from 10 ng/μl, 20 ng/μl, 30 ng/μl, 40 ng/μl, and 50 ng/μl.

### Mature RGC Maintenance (Day 37 on)

From Day 37 on: Medium can be changed twice a week with RGC induction media containing: 3 μM DAPT, 10 μM Y27632. (FIG. 7G)

### Removal of Neuronal Progenitor Cells (NPCs)

In certain embodiments, neuronal progenitor cells may contaminate mature iPSC-RGC cultures either in RGCs isolated from 3D-retinal organoids or using 2D-RGC cultures. To maintain long term cultures and use these cultures in in vitro studies, growth of neuronal progenitor cells should be minimized or eliminated.

We optimized compounds to enrich long term cultures of iPSC-RGCs by treatment with mitotic inhibitors like AraC-Cytosine β-D-arabinofuranoside, Sigma (0.05 μM to 10 μM) and Aphidicolin (Aphi) (0.05 μM-2 μM), treatment with polyIC peptide and/or transduction with AAV virus with eGFP in culture to prevent the growth of neuronal progenitor cells. Treatment of these compounds at Days 26-37 and Days 36-44 improved RGC purity and assisted long term invitro iPSC-RGC cultures.

### Flow Cytometry Analysis of RGCs

iPSC-RGC cultures were lifted using TrypLE (Invitrogen) and collected by centrifugation at 1600 rpm for 5 minutes at 4° C. The pelleted RGCs were resuspended in 1×HBSS and run through a 100 μm filter to ensure single cell suspension. A portion of cells were incubated with various antibodies anti-CD90-PE-Cy7 (Thy1), anti-Brn-3b Alexa Fluor 647, and anti-RBPMS Alexa Fluor 647 in 1% PBS supplemented with 0.5% bovine serum albumin plus 0.1% Na-Azide (FACS buffer) and incubated at room temperature (RT) for 40 minutes on ice at 4° C. as previously described.⁴¹ We analyzed stained cells using AccuriC6 and data analyzed using FlowJo Version 10.0.8 software (TreeStar).

### Immunohistochemistry

Immunohistochemical analysis was performed as previously published (Duong et al. 2018). For immunolocalization of antigens, RGCs plated on 14 mm coverslips were fixed in 4% paraformaldehyde followed by permeabilization using 0.1% Triton X-100 for 5 minutes. Cells were incubated for 1 hour at RT with a blocking buffer (1% BSA, 5% normal goat serum, 0.1% Triton X-100 in 1% PBS). RGCs were incubated with primary antibodies diluted in 1% BSA, 0.1% Triton X-100 in 1% PBS buffer for overnight at 4° C. After washing thrice with 1×PBS, cells were incubated with secondary antibodies diluted in 1% BSA, 0.1% Triton X-100 in 1% PBS buffer for 1 hour at RT. The coverslips were then mounted on slides using prolong Gold mounting medium with DAPI (4′,6-diamidino-2-phenylindole) (Invitrogen). RGCs immunohistochemistry was performed as described earlier 36-39 using SOX2, PAX6, RAX, Tuj1, Thy1/CD90, MAP2, GFAP, CRALBP, Bm-3a, and RBPMS antibodies with dilution as provided in Tables 3-5. Slides were observed under an Olympus FV1000 Confocal microscope and images were captured with the use of appropriate filters and lasers.

TABLE 3
Antibodies, their source and concentration used in Example 1

| Protein | Antibody | Clone/ | | Concentration/ | | | |
|---|---|---|---|---|---|---|---|
| species | Source | Catalog No | Species | Volume | | Dilution | Application |
| SOX2 | Cell | 3579S | Rabbit | | | 1/100 | ICC |
| | Signaling | | | | | | |
| | Technology | | | | | | |
| PAX6 | Biolegend | PRB-278P100 | Rabbit | 2 | mg/ml | 1/100 | ICC |
| RAX | Abcam | ab23340 | Rabbit | | | 1/100 | ICC |
| THY1/CD90 | R&D | AF2067 | Sheep | 25 | μg | 1/100 | ICC, How |
| | Systems | | | | | | cytometry |
| BRN3b | Santa cruz | sc-514474 | Mouse | 200 | μg | 1 μg/10⁶ cells | Flow |
| | | | | | | | cytometry |
| RBPMS | Novus | NBP2- | Mouse | 0.64 | mg/ml | 1 μL/10⁶ cells | Flow |
| | Biologicals | 73835AF647 | | | | | cytometry |
| TUJ1 | Biolegend | MRB-435P | Rabbit | 1 | mg/ml | 1/250 | ICC |
| RBPMS | Sigma- | ABN1362 | Rabbit | 100 | μg | 1/1500 | ICC |
| | Aldrich | | | | | | |
| MAP2 | Sigma | M1406 | Mouse | 2 | mg/ml | 1/500 | ICC |
| BRN3a | Santa cruz | sc-8429 | Mouse | 200 | μg/ml | 1/100 | ICC |
| BRN3b | Santa cruz | sc-6026 | Goat | 100 | μg/ml | 1/100 | ICC |
| TUBB3 | Biolegend | PRB-435P | Rabbit | 1.0 | mg/ml | 1/100 | ICC |
| CRALBP | Abcam | ab15051 | Mouse | 1.0 | mg/ml | 1/1000 | ICC |
| GFAP | Abcam | ab7260 | Rabbit | | | 1/1000 | ICC |
| SSEA4-APC | Biolegend | MC-813-70 | Mouse | 25 | μg/ml | 1/100 | Flow |
| | | | | | | | cytometry |
| BRN3a | Santa cruz | sc-31984 | Goat | 100 | μg | 1/100 | ICC (after |
| | | | | | | | MACS) |
| MAP2 | Santa cruz | sc-74421 | Mouse | 200 | μg | 1/100 | ICC (after |
| | | | | | | | MACS) |
| GFAP | Abcam | ab7260 | Rabbit | 50 | μl | 1/1000 | ICC (after |
| | | | | | | | MACS) |
| Alexa Fluor | Invitrogen | A11020 | Goat | 500 | μg | 1/500 | ICC |
| 594 Goat | | | | | | | |
| Anti-Mouse | | | | | | | |
| Alexa Fluor | Invitrogen | A21467 | Chicken | 2 | mg/mL | 1/500 | ICC |
| 488 Chicken | | | | | | | |
| anti- Goat | | | | | | | |
| Alexa Fluor | Invitrogen | A11034 | Goat | 1 | mg | 1/500 | ICC |
| 488 Goat | | | | | | | |
| anti- Rabbit | | | | | | | |

*ICC—Immunocytochemistry

TABLE 4
Small molecules and protein source information

| Small molecules/recombinant | | | |
|---|---|---|---|
| protein | Company | Cat No. | Reconstitution |
| Recombinant Human FGF | R & D Systems | 233-FB | 0.1% BSA in PBS |
| basic (146 aa) Protein | | | |
| Recombinant Human | R & D Systems | 8908-SH | 0.1% BSA in PBS |
| Sonic Hedgehog/Shh | | | |
| Protein | | | |
| Recombinant Human/Mouse | R & D Systems | 423-F8 | 0.1% BSA in PBS |
| FGF-8b Protein | | | |
| DAPT | Stemgent | 04-0041 | DMSO |
| Recombinant Human | R & D Systems | 669-FO | 0.1% BSA in PBS |
| Follistatin 300 Protein | | | |
| Cyclopamine | R & D Systems | 1623/1 | DMSO |
| 0BRecombinant Human BDNF | R & D Systems | 248-BDB | 0.1% BSA in PBS |
| Protein | | | |
| Forskolin | Selleckchem | S2449 | |
| Recombinant Human | R & D Systems | 268-N4 | 0.1% BSA in PBS |
| NT-4 Protein | | | |
| Recombinant Human | R & D Systems | 257-NT | 0.1% BSA in PBS |
| CNTF Protein | | | |
| cAMP | Sigma-aldrich | D0627 | DMSO |
| N⁶,2′-O- | | | |
| Dibutyryladenosine 3′,5′- | | | |
| cyclic monophosphate | | | |
| sodium salt | | | |
| Y-27632 | R & D Systems | 1254/1 | DMSO |
| dihydrochloride | | | |
| LDN 193189 | R & D Systems | 6053 | DMSO |
| dihydrochloride | | | |
| SB 431542 | R & D Systems | 1614 | DMSO |
| CHIR 99021 | Selleckchem | S2924 | DMSO |
| XAV 939 | R & D Systems | 3748 | DMSO |
| Nicotinamide | Sigma-aldrich | N0636 | DMSO |
| IGF | R & D Systems | 291-G1 | 0.1% BSA in PBS |
| CHIR99021 | Selleckchem | S2924 | DMSO |

TABLE 5
Media and reagents used in Example 1

| Media/Reagent | Product | Catalog no. |
|---|---|---|
| DMEM/F12 + Glutamine + Hepes | Corning | 10-092-cm |
| 1 x Penn/Strep | Life Technologies | 10378-016 |
| 1 x Glutamine MAX | Life Technologies | 35050-061 |
| NEAA | Life Technologies | 11140-050 |
| 2ME | Life Technologies | 21985-023 |
| N2 supplement | Life Technologies | 17502-048 |
| B27 Supplement without Vitamin A | Life Technologies | 12587-010 |
| Neurolbasal | Life Technologies | 21103049 |
| TrypLE | Life Technologies | 12605-010 |

### Voltage Recordings

The circular cover glass coated with 1:100 growth factor reduced matrigel containing adherent cells was transferred to the recording chamber filled with Neurobasal media supplemented with 2% B27 without vitamin-A and 1% Glutamax. The chamber was placed on the microscope stage (Olympus BX-61 microscope) and perfused with oxygenated Ames's solution (Sigma-Aldrich) resulting in gradual replacement of the growth medium with the Ames medium. The temperature of the solution inside the chamber was gradually increased to 37° C. prior to imaging and recording session using Warner Instruments TC 344B. Confocal images of the cells were acquired with Olympus Fluoview 1000 MPE system and cells demonstrating strong GFP expression were selected for patch-clamp recording. Whole cell configuration was achieved in the voltage-clamp mode at −60 mV holding potential. After achieving whole-cell configuration, cells were either maintained in the current clamp mode at zero holding current and depolarized with calibrated current steps, or in the voltage-clamp mode at −60 mV holding potential from which they were depolarized with voltage steps. Warner Instruments PC 505B amplifier and Molecular Devices Digidata 1440 digitizer under the control of the Clampex software were used for recording of the membrane voltage and current. Patch pipettes (1.2/0.69 mm) were filled with (in mM) 110 K-gluconate, 12 NaCl, 10 HEPES, 1 EGTA solution. Pipettes were mounted on Sutter MPC-200 micromanipulators; MTI CCD 72 camera system was used to provide video control over pipette and cell positioning in the chamber.

### Quantitative Real-Time PCR

Total RNA was extracted using Purelink RNA isolation kit (invitrogen). For each sample, 1 μg of total RNA was reverse transcribed using the SuperScript III first stand cDNA synthesis kit with random primers (ThermoFisher). Amplified cDNA was used to quantify Taqman probes (target-FAM and housekeeping-TET) and TaqMan Fast advanced Master Mix (ThermoFisher) on a 7900 Fast Real-time PCR system (Applied Biosystems). All results were normalized to a B2M housekeeping control and were from 3 technical replicates of 3 independent biological samples for each time-point and experimental condition.

### Magnetic Activated Cell Sorting (MACS) to Purify CD90+ve RGCs

RGC cells were lifted using TrypLE (Invitrogen), pelleted by centrifugation at 350 g for 5 minutes, and total cell number was determined. Cell pellet was resuspended in 90 μL buffer (1×PBS pH 7.2, 0.5% BSA, and 2 mM EDTA) and 10 μL of CD90.2 microbeads (catalog #130-121-278, Miltenyi Biotec) per 107 total cells. Cell suspension was mixed well and incubated at RT for 15 min in a tube rotator. In the meantime, MS column was placed onto a MACS separator and the column was prepped. Following the 15 min incubation, the cell suspension was applied onto the column. Flow-through from the column represented the unlabeled or CD90.2− cell fraction. The column was washed with appropriate volume of buffer for at least twice. The column was then removed from the separator and placed on a suitable collection tube. Appropriate volume of buffer was added to the column and magnetically labeled CD90.2+ cells were immediately flushed out by firmly pushing the plunger into the column. The cells were plated using RGCs induction media containing 3 μM DAPT and 10 μM ROCK inhibitor.

### Statistical Analysis

Quantitative data were obtained from three independent experiments per cell line in triplicate. Statistical analysis was performed with Student T-test in Prism. *p-values of <0.05 were considered statistically significant.

### Results for Example 1

Combined Inhibition of Wnt, BMP, TGF-β and Nicotinamide Contributes to Efficient Generation of Early RPCs.

hiPSC retinal differentiation methods can differ between 2D and 3D culture conditions, and the use of proteins and small molecules to recapitulate processes responsible for vertebrate retina development have been studied.^{26,40} Previously, the generation of RGCs from hPSCs required that cells be maintained on matrigel as an adherent support to culture cells obtained from 3D cell aggregates and several additive factors like Noggin, DKK-1, IGF-1, bFGF2, and proneural supplements.^{41,42,43} Teotia et al, successfully used a modified version of the “Lamba protocol” by differentiating hiPSCs initially into neural rosettes, manual picking, and then induction to functional RGCs, by recapitulating retinal developmental pathways using chemical regulators.^{26,41} These conditions require mid-differentiation enrichment steps that involves the establishment of retinal rosettes followed by manual isolation and maturation to generate retinal subtypes. CRISPR engineered iPSC lines containing fluorescent RFP reporter tag in the Brn3B locus, greatly assisted in evaluation of pathways necessary for RGC differentiation and characterization.⁴⁴ This methodology provided a protocol which utilized a monolayer cultures with defined factor supplementations; however, the evaluation were only performed using human embryonic stem cells (hESCs) and resulted in proportions of RGCs between 20-30% of the overall retinal differentiation.

A major challenge in the regenerative medicine and disease modeling field are the reproducibility between experiments, and variation between individual to individual. Therefore, we set out to develop and characterize a modified two-stage protocol that differentiates hiPSCs into an enriched population of retinal progenitor cell (RPC) cultures followed by targeted differentiation to RGCs that is reproducible, efficient, and requires minimal personnel interpretation in RGCs generation and maintenance.²⁶ To accomplish this, hiPSCs were grown to confluence and subsequently treated with a RPC induction media containing: DMEM/F12 plus N2, B27, XAV939 (WNT inhibitor), SB431542 (TGF-β inhibitor), LDN193189, (BMP inhibitor), nicotinamide, and IGF1 for 4 days (FIG. 1A). The inhibition of Wnt and BMP signaling has been documented to enhance the expression of eye field transcription factors (EFTFs) during retinal differentiations of hiPSC. We observed that addition of TGF-β inhibition induced greater EFTFs expression during early retinal differentiation. Nicotinamide was added to the differentiation media (DO-D3) to promote the expression of early eye field markers LHX2 and RAX. Nicotinamide has been shown to promote cell expansion and adaptation to a radial/rosette morphology. Differentiation factors such as IGF-1 and bFGF2 aid in the specification of eye field identity to differentiating retinal progenitors.²⁷ From Day 4-21, nicotinamide was removed and bFGF was added to RPC induction media. Analysis at day 7 showed a uniform population of SOX2, RAX and PAX6 positive cells (FIG. 1A). The expression of early retinal progenitor markers, LHX2 and RAX, were identified in over 95% of day 7 cultures (FIG. 1B) indicating an efficient and robust generation of RPCs. Quantification of EFTFs, LHX2, RAX, PAX6, SIX6, SIX3, and VSX2, showed significant expression relative to pluripotent stem cells in all three individual cell lines (FIG. 1C).

The efficient induction of retinal progenitors is largely dependent on the inhibition of pathways responsible for patterning of diencephalon; however, it was previously unclear if modification of these inhibitory pathways play a role on retinal ganglion cell generation. Here, we tested whether pharmacological inhibition of Wnt, TGF-β, and BMP can improve RGC induction and subsequently enable the controlled generation of mature iPSC-RGC neurons. We evaluated seven different treatment conditions to determine pathways that enhance or restrict RPC generation from iPSCs. The following 7 conditions were tested from d16-d21: 1) BMP (L), 2) Wnt inhibition (X), 3) CHIR (Wnt agonist), 4) LX, 5) BMP and TGF-β (LSB), 6) LSB-CHIR and LSBX (Schematic in FIG. 1A).

Extended Exposures of BMP, Wnt, and TGF-β Inhibition Leads to Efficient Generation of RGC Neurons.

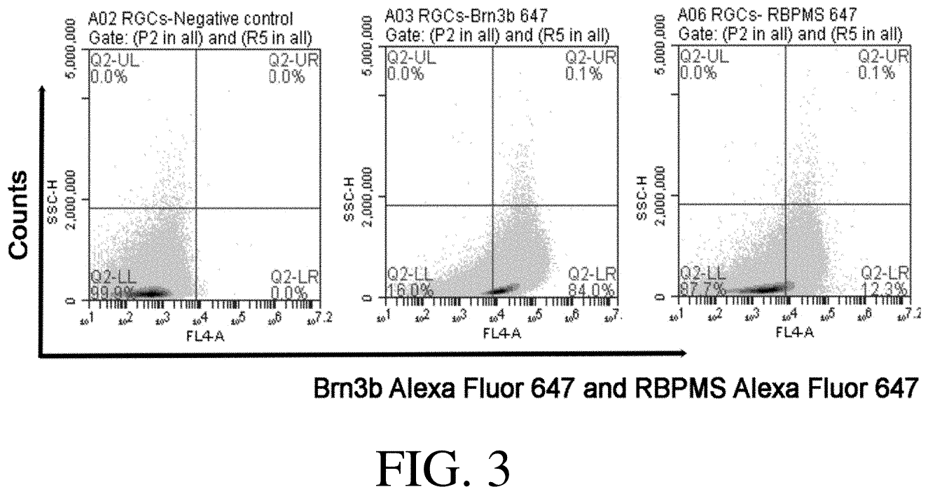

RGC differentiation proceeded through two phases: 1) uniform RPC cultures (up to D21), and 2) late stage RGC differentiation (D22-36) (FIG. 2A,B). At day 22-23, cells were treated with RGC induction conditions that included the activation of sonic hedgehog, by augmenting with SHH (250 ng/mL). Comparative studies in our lab showed the use of SAG (100 nM, smoothened agonist) could replace SHH with no difference in RGC induction changes. Fibroblast growth factor 8 (FGF8) and notch inhibitor (DAPT) were added along with SHH or SAG. During iPSC generation, SHH pathway signaling was shown to enhance the specification of RPCs to RGCs during this 48 h time frame. On day 24, cultures were expanded using a cross-hatching technique known separate cells into small clusters and SHH, TGF-β, and notch signaling were inhibited to promote RGC maturation using cyclopamine, follistatin, and DAPT, cyclopamine was removed for the following 2 days. To improve survival of RGCs during the expansion, cAMP, BDNF, neurotrophin 4 (NT4) and CNTF, were added to the media to promote the development and survival of cells with neuronal fate. Forskolin and Y-27632 were included as compounds shown to promote RGC neurite growth, survival and lineage commitment. After 35 days in vitro, cultures were disassociated and flow cytometric analysis were performed on single cell suspension obtained from three independent iPSC lines, and data showed that highest proportions of RGCs were generated from the LSBX conditions, with a range between 40-50% Thy-1 (CD90) (FIG. 2C, E), 82-84% Brn-3b, and 11-12% RBPMS (FIG. 3). The LX, CHIR, and LSB conditions resulted in 26.7% (±3.2), 22.5% (±4.7), and 27.2% Thy-1 positive cells (±6.2), respectively (FIG. 2C). We observed consistent results across individuals and experiments (FIG. 2D).

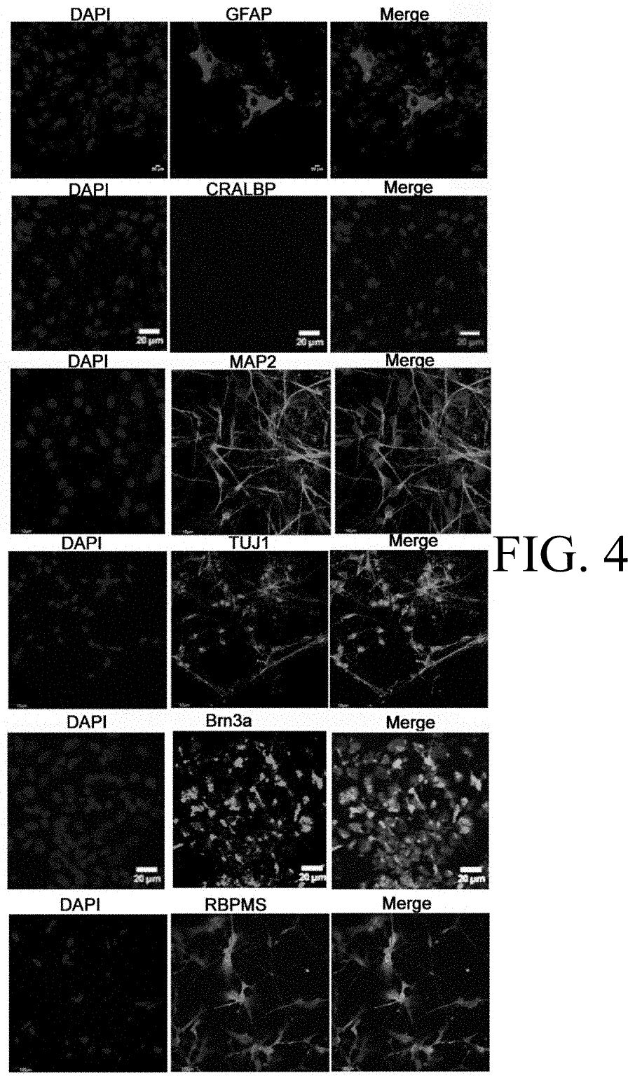

Additionally, on Day 35 we further concentrated the number of RGCs in our culture by employing the MACS technique and CD90.2 microbeads. Since RPCs are multipotent cells, they have the potential to differentiate into neuronal cell types and one glial cell type called the Müller glial cells^{61,62}. Therefore, we utilized the CD90.2 microbeads for the positive selection of RGCs expressing Thy1 cell surface marker and the removal of Müller glial cells from our culture. Following the purification of RGCs using MACS, the cells were analyzed using immunocytochemistry by detecting for the presence of Müller glial cells, astrocytes, and RGCs using CRALBP, GFAP, MAP2 TUJ1, RBPMS and Brn-3a markers, respectively (FIG. 4). Our results show that roughly 95% of the cells in our culture were positive for Brn-3a with the presence of extended synaptic connections between RGCs. Whereas, about 5% of the cells were positive for astrocytes and we detected no presence of Müller glial cells in our system (FIG. 4). However, the presence of astrocytes is significant since they are essential for the functional activity of the neuronal cell types by ensuring proper synaptic maturation and signaling⁶³.

For long-term cultures, we initially used RGC induction media supplemented with 1% N₂ and 1% B27 for the maintenance of RGC cultures after DIV35; however, this resulted in significant overgrowth of proliferative late RPCs. The removal of N₂ supplementation reduced the number of dividing cells in our cultures, providing a culture with predominantly RGCs. We have also cultured RGCs in 1% CultureOne supplement (Life Technologies) in the RGC maturation media, which reduced the number of non-RGC cell growth in day 45 (only in two weeks, from Day 27-40) cultures and beyond. After Day 35 in culture, the iPSC derived RGCs produce appropriate morphological and physiological features of mature RGCs.

Gene Expression Profiles During Differentiation of iPSC to RPCs and RGCs:

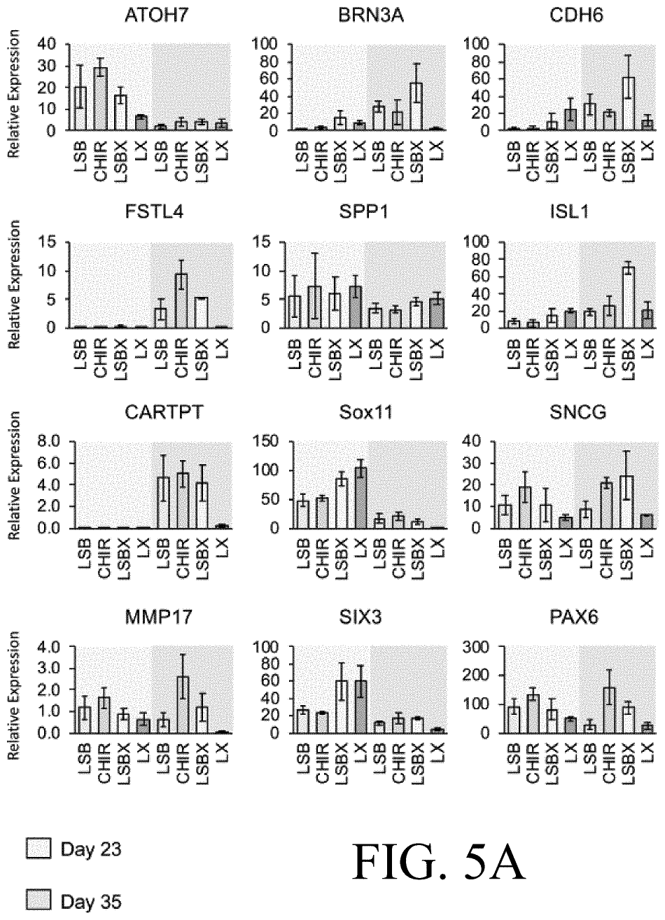

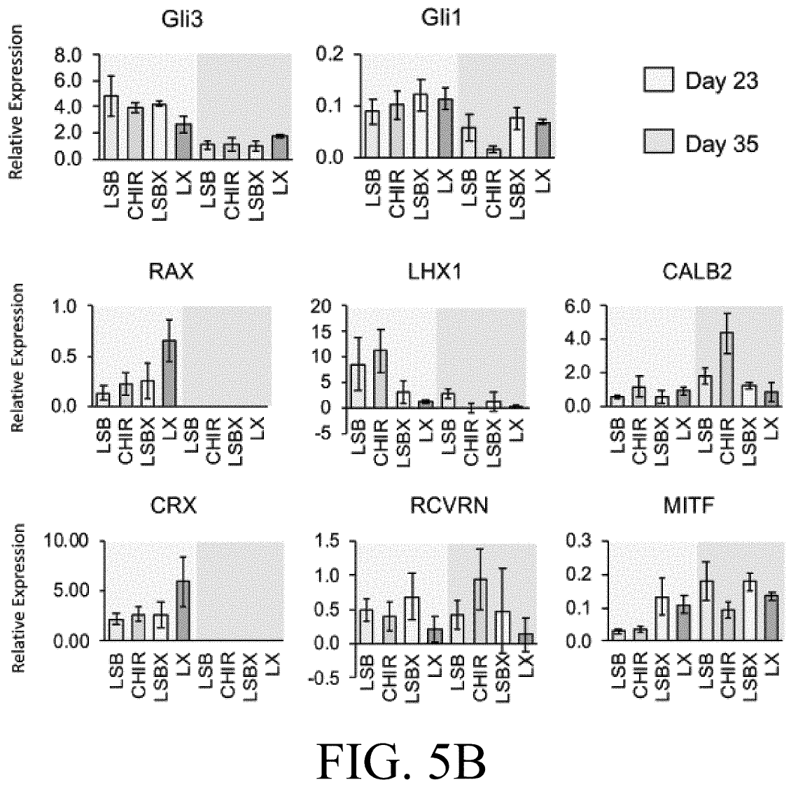

During eye formation in vertebrates, cell intrinsic signals, extrinsic signals and/or transcription factors control the differentiation and fate determination of retinal cells. We evaluated the gene expression profile of three EFTs, RAX, PAX6, and SIX3 that play a role in the anterior neural plate (FIG. 5). The expression of Rx (encoded by RAX gene) was maximum in the RPC inhibited by BMP and Wnt inhibition when compared to the other conditions at DIV23 (FIG. 5, Table 6).

TABLE 6

| Detector | | | | |
|---|---|---|---|---|
| Name | Conditions | ΔΔCt | p value | Fold change |
| ATOH7 | L vs LSB | 0.962 | 0.012 | 0.513 |
| | L vs LSBX | 0.409 | 0.185 | 0.753 |
| | L vs LSB-CHIR | 0.435 | 0.008 | 0.740 |
| | X vs LSB | 0.308 | 0.094 | 0.808 |
| | X vs LSBX | −0.246 | 0.352 | 1.186 |
| | X vs LSB-CHIR | −0.220 | 0.034 | 1.165 |
| | LSB vs LSBX | −0.553 | 0.133 | 1.467 |
| | LSB vs LSB-CHIR | −0.527 | 0.034 | 1.441 |
| | LSBX vs LSB-CHIR | 0.026 | 0.910 | 0.982 |
| BRN3a | L vs LSB | −0.417 | 0.067 | 1.335 |
| | L vs LSBX | −1.471 | 0.003 | 2.772 |
| | L vs LSB-CHIR | −1.866 | 0.008 | 3.645 |
| | X vs LSB | 0.026 | 0.888 | 0.982 |
| | X vs LSBX | −1.029 | 0.018 | 2.040 |
| | X vs LSB-CHIR | −1.423 | 0.016 | 2.682 |
| | LSB vs LSBX | −1.054 | 0.010 | 2.077 |
| | LSB vs LSB-CHIR | −1.449 | 0.016 | 2.730 |
| | LSBX vs LSB-CHIR | −0.395 | 0.141 | 1.315 |
| CALB2 | L vs LSB | −0.849 | 0.018 | 1.801 |
| | L vs LSBX | −1.554 | 0.000 | 2.937 |
| | L vs LSB-CHIR | −1.477 | 0.002 | 2.783 |
| | X vs LSB | 1.000 | 0.027 | 0.500 |
| | X vs LSBX | 0.295 | 0.149 | 0.815 |
| | X vs LSB-CHIR | 0.372 | 0.036 | 0.773 |
| | LSB vs LSBX | −0.705 | 0.025 | 1.630 |
| | LSB vs LSB-CHIR | −0.628 | 0.036 | 1.545 |
| | LSBX vs LSB-CHIR | 0.078 | 0.304 | 0.948 |
| CARTPT | L vs LSB | 0.860 | 0.006 | 0.551 |
| | L vs LSBX | 0.733 | 0.010 | 0.602 |
| | L vs LSB-CHIR | −0.864 | 0.005 | 1.820 |
| | X vs LSB | 0.942 | 0.040 | 0.520 |
| | X vs LSBX | 0.815 | 0.053 | 0.568 |
| | X vs LSB-CHIR | −0.782 | 0.000 | 1.719 |
| | LSB vs LSBX | −0.127 | 0.075 | 1.092 |
| | LSB vs LSB-CHIR | −1.724 | 0.000 | 3.303 |
| | LSBX vs LSB-CHIR | −1.597 | 0.000 | 3.025 |
| CDH6 | L vs LSB | −0.215 | 0.371 | 1.160 |
| | L vs LSBX | −0.672 | 0.090 | 1.593 |
| | L vs LSB-CHIR | −1.911 | 0.012 | 3.761 |
| | X vs LSB | 0.049 | 0.859 | 0.967 |
| | X vs LSBX | −0.408 | 0.264 | 1.327 |
| | X vs LSB-CHIR | −1.648 | 0.004 | 3.133 |
| | LSB vs LSBX | −0.457 | 0.064 | 1.373 |
| | LSB vs LSB-CHIR | −1.697 | 0.004 | 3.241 |
| | LSBX vs LSB-CHIR | −1.240 | 0.015 | 2.361 |
| CRX | L vs LSB | −2.150 | 0.003 | 4.437 |
| | L vs LSBX | −1.928 | 0.020 | 3.806 |
| | L vs LSB-CHIR | −2.775 | 0.005 | 6.843 |
| | X vs LSB | −2.550 | 0.002 | 5.855 |
| | X vs LSBX | −2.329 | 0.014 | 5.023 |
| | X vs LSB-CHIR | −3.175 | 0.081 | 9.031 |
| | LSB vs LSBX | 0.221 | 0.501 | 0.858 |
| | LSB vs LSB-CHIR | −0.625 | 0.081 | 1.542 |
| | LSBX vs LSB-CHIR | −0.846 | 0.118 | 1.798 |
| FSTL4 | L vs LSB | −0.591 | 0.011 | 1.506 |
| | L vs LSBX | −0.665 | 0.012 | 1.585 |
| | L vs LSB-CHIR | −1.344 | 0.024 | 2.539 |
| | X vs LSB | 0.642 | 0.081 | 0.641 |
| | X vs LSBX | 0.568 | 0.104 | 0.675 |
| | X vs LSB-CHIR | −0.112 | 0.074 | 1.081 |
| | LSB vs LSBX | −0.074 | 0.491 | 1.053 |
| | LSB vs LSB-CHIR | −0.754 | 0.074 | 1.686 |
| | LSBX vs LSB-CHIR | −0.680 | 0.091 | 1.602 |
| Gli1 | L vs LSB | −1.790 | 0.006 | 3.459 |
| | L vs LSBX | −1.195 | 0.002 | 2.289 |
| | L vs LSB-CHIR | −2.035 | 0.002 | 4.098 |
| | X vs LSB | −0.345 | 0.139 | 1.270 |
| | X vs LSBX | 0.250 | 0.069 | 0.841 |
| | X vs LSB-CHIR | −0.590 | 0.278 | 1.505 |
| | LSB vs LSBX | 0.595 | 0.054 | 0.662 |
| | LSB vs LSB-CHIR | −0.245 | 0.278 | 1.185 |
| | LSBX vs LSB-CHIR | −0.840 | 0.016 | 1.790 |
| Gli3 | L vs LSB | −0.597 | 0.004 | 1.513 |
| | L vs LSBX | −0.980 | 0.025 | 1.972 |
| | L vs LSB-CHIR | −1.562 | 0.005 | 2.953 |
| | X vs LSB | 0.118 | 0.234 | 0.922 |
| | X vs LSBX | −0.265 | 0.255 | 1.201 |
| | X vs LSB-CHIR | −0.847 | 0.014 | 1.799 |
| | LSB vs LSBX | −0.383 | 0.136 | 1.304 |
| | LSB vs LSB-CHIR | −0.965 | 0.014 | 1.952 |
| | LSBX vs LSB-CHIR | −0.582 | 0.094 | 1.497 |
| ISL1 | L vs LSB | −0.095 | 0.417 | 1.068 |
| | L vs LSBX | −0.264 | 0.063 | 1.201 |
| | L vs LSB-CHIR | −1.012 | 0.002 | 2.016 |
| | X vs LSB | −0.426 | 0.065 | 1.343 |
| | X vs LSBX | −0.595 | 0.025 | 1.511 |
| | X vs LSB-CHIR | −1.343 | 0.008 | 2.537 |
| | LSB vs LSBX | −0.169 | 0.225 | 1.125 |
| | LSB vs LSB-CHIR | −0.917 | 0.008 | 1.888 |
| | LSBX vs LSB-CHIR | −0.748 | 0.005 | 1.679 |
| LHX2 | L vs LSB | −2.009 | 0.009 | 4.025 |
| | L vs LSBX | −0.911 | 0.033 | 1.880 |
| | L vs LSB-CHIR | −1.243 | 0.018 | 2.368 |
| | X vs LSB | −1.826 | 0.024 | 3.546 |
| | X vs LSBX | −0.728 | 0.118 | 1.657 |
| | X vs LSB-CHIR | −1.061 | 0.016 | 2.086 |
| | LSB vs LSBX | 1.098 | 0.008 | 0.467 |
| | LSB vs LSB-CHIR | 0.765 | 0.016 | 0.588 |
| | LSBX vs LSB-CHIR | −0.333 | 0.022 | 1.259 |
| MMP17 | L vs LSB | −1.903 | 0.013 | 3.741 |
| | L vs LSBX | −1.883 | 0.014 | 3.687 |
| | L vs LSB-CHIR | −2.266 | 0.009 | 4.811 |
| | X vs LSB | −1.373 | 0.025 | 2.590 |
| | X vs LSBX | −1.352 | 0.027 | 2.553 |
| | X vs LSB-CHIR | −1.736 | 0.043 | 3.331 |
| | LSB vs LSBX | 0.021 | 0.842 | 0.986 |
| | LSB vs LSB-CHIR | −0.363 | 0.043 | 1.286 |
| | LSBX vs LSB-CHIR | −0.384 | 0.054 | 1.305 |
| RCVRN | L vs LSB | −0.764 | 0.223 | 1.698 |
| | L vs LSBX | −2.338 | 0.017 | 5.055 |
| | L vs LSB-CHIR | −4.277 | 0.041 | 19.388 |
| | X vs LSB | −1.798 | 0.079 | 3.477 |
| | X vs LSBX | −3.372 | 0.016 | 10.351 |
| | X vs LSB-CHIR | −5.311 | 0.072 | 39.704 |
| | LSB vs LSBX | −1.574 | 0.097 | 2.977 |
| | LSB vs LSB-CHIR | −3.513 | 0.072 | 11.419 |
| | LSBX vs LSB-CHIR | −1.939 | 0.177 | 3.836 |
| SIX3 | L vs LSB | −0.163 | 0.079 | 1.120 |
| | L vs LSBX | −0.639 | 0.006 | 1.557 |
| | L vs LSB-CHIR | −0.990 | 0.003 | 1.986 |
| | X vs LSB | 0.215 | 0.243 | 0.862 |
| | X vs LSBX | −0.260 | 0.184 | 1.198 |
| | X vs LSB-CHIR | −0.612 | 0.001 | 1.528 |
| | LSB vs LSBX | −0.475 | 0.001 | 1.390 |
| | LSB vs LSB-CHIR | −0.827 | 0.001 | 1.774 |
| | LSBX vs LSB-CHIR | −0.351 | 0.004 | 1.276 |
| SNCG | L vs LSB | −0.351 | 0.235 | 1.276 |
| | L vs LSBX | −0.354 | 0.036 | 1.278 |
| | L vs LSB-CHIR | −0.411 | 0.025 | 1.330 |
| | X vs LSB | 0.102 | 0.737 | 0.932 |
| | X vs LSBX | 0.099 | 0.629 | 0.933 |
| | X vs LSB-CHIR | 0.042 | 0.794 | 0.971 |
| | LSB vs LSBX | −0.003 | 0.991 | 1.002 |
| | LSB vs LSB-CHIR | −0.060 | 0.794 | 1.042 |
| | LSBX vs LSB-CHIR | −0.057 | 0.212 | 1.040 |
| Sox11 | L vs LSB | −2.023 | 0.005 | 4.063 |
| | L vs LSBX | −1.797 | 0.003 | 3.474 |
| | L vs LSB-CHIR | −1.833 | 0.013 | 3.564 |
| | X vs LSB | −2.054 | 0.002 | 4.152 |
| | X vs LSBX | −1.828 | 0.000 | 3.550 |
| | X vs LSB-CHIR | −1.865 | 0.458 | 3.642 |
| | LSB vs LSBX | 0.226 | 0.157 | 0.855 |
| | LSB vs LSB-CHIR | 0.189 | 0.458 | 0.877 |
| | LSBX vs LSB-CHIR | −0.037 | 0.861 | 1.026 |
| SPP1 | L vs LSB | −0.986 | 0.094 | 1.981 |
| | L vs LSBX | 0.086 | 0.816 | 0.942 |
| | L vs LSB-CHIR | −1.270 | 0.076 | 2.412 |
| | X vs LSB | −1.127 | 0.007 | 2.183 |
| | X vs LSBX | −0.054 | 0.628 | 1.038 |
| | X vs LSB-CHIR | −1.411 | 0.255 | 2.659 |
| | LSB vs LSBX | 1.072 | 0.001 | 0.475 |
| | LSB vs LSB-CHIR | −0.284 | 0.255 | 1.218 |
| | LSBX vs LSB-CHIR | −1.357 | 0.017 | 2.561 |
| RAX | L vs LSB | −3.264 | 0.000 | 9.607 |
| | L vs LSBX | −1.326 | 0.027 | 2.507 |
| | L vs LSB-CHIR | −1.526 | 0.002 | 2.880 |
| | X vs LSB | −2.416 | 0.000 | 5.336 |
| | X vs LSBX | −0.478 | 0.167 | 1.392 |
| | X vs LSB-CHIR | −0.678 | 0.002 | 1.600 |
| | LSB vs LSBX | 1.938 | 0.013 | 0.261 |
| | LSB vs LSB-CHIR | 1.738 | 0.002 | 0.300 |
| | LSBX vs LSB-CHIR | −0.200 | 0.473 | 1.149 |
| MITF | L vs LSB | −1.349 | 0.001 | 2.548 |
| | L vs LSBX | −1.335 | 0.002 | 2.523 |
| | L vs LSB-CHIR | −1.567 | 0.002 | 2.963 |
| | X vs LSB | −0.355 | 0.014 | 1.279 |
| | X vs LSBX | −0.340 | 0.018 | 1.266 |
| | X vs LSB-CHIR | −0.572 | 0.107 | 1.487 |
| | LSB vs LSBX | 0.014 | 0.837 | 0.990 |
| | LSB vs LSB-CHIR | −0.218 | 0.107 | 1.163 |
| | LSBX vs LSB-CHIR | −0.232 | 0.099 | 1.175 |
| PAX6 | L vs LSB | −1.015 | 0.003 | 2.021 |
| | L vs LSBX | −0.960 | 0.003 | 1.945 |
| | L vs LSB-CHIR | −1.303 | 0.010 | 2.467 |
| | X vs LSB | −0.291 | 0.028 | 1.224 |
| | X vs LSBX | −0.236 | 0.031 | 1.178 |
| | X vs LSB-CHIR | −0.579 | 0.174 | 1.493 |
| | LSB vs LSBX | 0.055 | 0.488 | 0.962 |
| | LSB vs LSB-CHIR | −0.287 | 0.174 | 1.220 |
| | LSBX vs LSB-CHIR | −0.343 | 0.129 | 1.268 |

The RAX expression at DIV35 RGCs was minimal suggesting a commitment to a more differentiated retinal fate, a consequence of retinal progenitor cell (RPC) expansion. PAX6 is expressed in the cornea, lens, ciliary body, and retina through development and plays a role in determining their cell fate. The PAX6 transcript expression was observed in all experimental conditions in RPCs and RGCs; however, predominant expression of PAX6 is detected at DIV23 and DIV35 in the CHIR condition (FIG. 5), which stimulates the canonical Wnt signaling. Our results indicate that prolonged stimulation of RPCs with Wnt restricts their differentiation potential and maintains majority of the cells as multipotent progenitors.

Among other gene transcripts analyzed, we observed an increase in SIX3 expression in both LX and LSBX conditions at DIV23 days, when compared to RPCs stimulated with LSB or CHIR. The expression of SIX3 decreased in RGCs at DIV35 indicating that neurospecification was reaching completion at this stage.

The SOX11 transcript is heavily expressed in developing retina during embryonic stages⁵⁰. It is required for the maintenance of hedgehog signaling and is critical for axonal growth, extension and driving adult neurogenesis⁵¹⁻⁵³. The expression of SOX11 significantly increased in LSBX and LX conditions when compared to LSB and CHIR in RPCs at DIV23. The expression of SOX11 was significantly reduced in RGCs at DIV35 (FIG. 5). GLI3 has a dual function as a transcriptional activator and a repressor of the sonic hedgehog (Shh) pathway. GLI1 is a simple transcriptional activator encoded by a target gene of Shh signaling. We observed increased expression of GLI3 in RPCs at DIV23 when compared to RGCs at DIV35. Expression of both GLI3 and GLI1 genes demonstrated that sonic hedgehog signaling is important for development of RGCs.

Direction selective RGC (DS-RGC) are subtypes of RGCs that respond to motion of light in different directions can be identified by expression of specific molecular markers, such as CART, CDH6, and FSTL4, among other genes.⁵⁴⁻⁵⁷ The CDH6 mediates axon-target matching and promotes wiring specificity that does not lead to image formation in the mammalian visual system. Cadherin mediated cell-cell adhesion ensures precise connectivity of neurons in the eye to target nuclei in the brain. Increased expression of CDH6 was observed in LSBX condition in DIV35 RGCs demonstrating their maturation towards increased specificity for axonal wiring between RGCs. The CARTPT (cocaine- and amphetamine-regulated transcript) is expressed by a major subtype of RGCs, ooDSGCs. Here, the expression of CARTPT was seen in RGCs matured only in LSB, CHIR and LSBX conditions at DIV35 indicating that these conditions develop specific subtypes of mature RGCs that are known to be markers for ON-OFF direction-selective RGCs.

FSTL4 is a gene expressed in ON DS-RGCs and is colocalized with BRN3B in few RGCs.⁴³ We have seen increased expression of FSTL4 gene transcript in CHIR induced RGCs when compared with other conditions at DIV35 indicating the development of various subtypes of mature RGCs in our culture conditions. We did not observe expression of CARTPT or FSTL4 transcripts in RPCs at DIV23 (FIG. 5).

The RGCs at DIV35 expressed BRN3A transcript predominantly in LSBX growth condition when compared to other conditions. Interestingly, ATOH7 expression was observed early in RPC differentiation at DIV23 and decreased in RGCs by DIV35. We detected low CRX expression levels in DIV35 RGC cultures across all culture conditions indicating that the RGC differentiation conditions at this stage restrict the photoreceptor precursor cell populations. We observed low expression of RCVRN (expressed by photoreceptors) and MITF in our cultures at DIV23 and 35 indicating that our cultures are differentiated predominantly towards RGC fate, with minimal retinal pigment epithelium cell identity (FIG. 5). Development of few interneurons in mature RGCs (amacrine or horizontal cells) was also observed, as evidenced by expression of CALB2 especially in RGCs with Wnt activation at DIV35.

To understand the importance and requirement of BMP, Wnt, TGF-β inhibition in differentiation and maintenance of RPCs, we performed pairwise gene expression analysis of the RPC cultures at DIV23 for the four conditions (L, SB, X and CHIR) in different combinations (Table 6). We observed increased expression of RAX, PAX6, LHX2 and SOX11 at DIV23 in L and X when compared to LSB, LSBX and LSB-CHIR indicating that BMP and Wnt inhibition is required for maintaining majority of cells in retinal progenitor state. High expression of CRX, and RCVRN at DIV23 in L, X and LSB when compared to LSB-CHIR indicates that Wnt signaling may play role in preventing the differentiation of RPCs toward photoreceptors lineage. Decreased expression of CALB2 at DIV23 in X (Wnt inhibitor) condition when compared to LSB, LSBX and LSB-CHIR; while increased expression at DIV35 under CHIR (Wnt agonist) condition indicates that Wnt signaling is crucial for promoting RPC differentiation to RGC. Increased expression of GLI1 and GLI3 transcripts at DIV23 in L (BMP inhibitor) condition when compared to LSB, LSBX and LSB-CHIR indicates that BMP inhibition is important for promoting SHH signaling (Table 6).

Functional Analysis of iPSC-RGCs

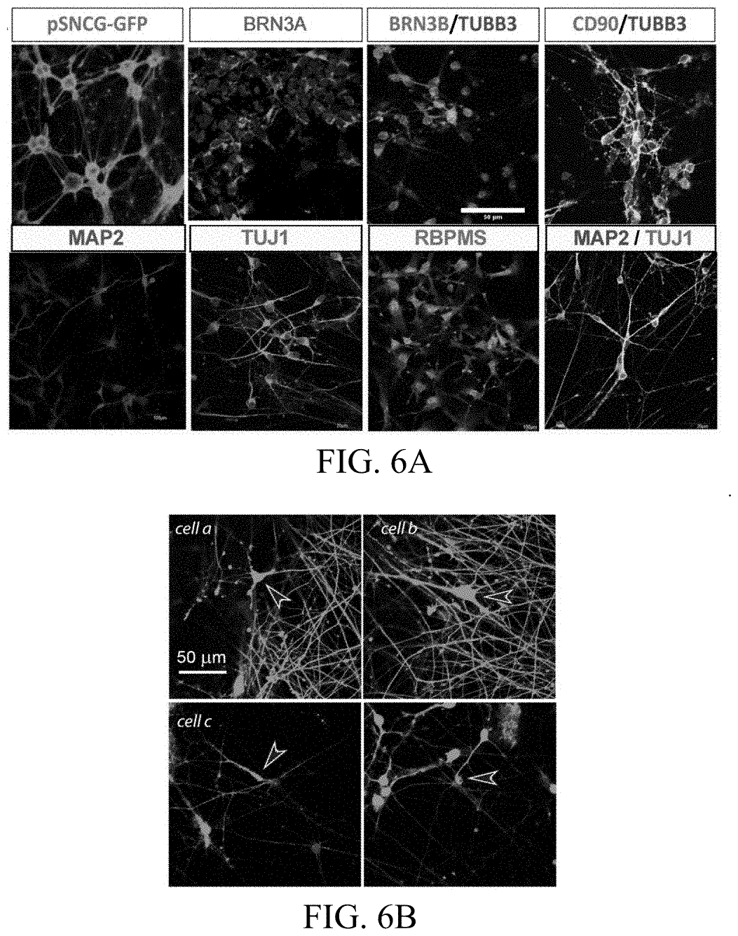

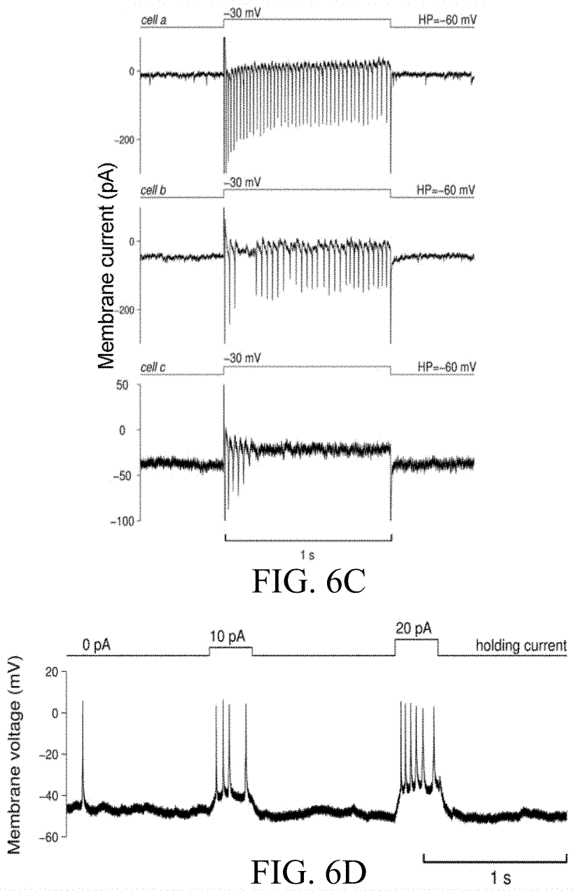

Confocal imaging of iPSC-RGC cultures at DIV35 show the presence of the GFP-expressing neurons with neurite projections, and an increase in the density and complexity of the projections over the period of one month. The iPSC-derived RGCs exhibited morphological features, with large somas connected by elongated axonal processes. Immunocytochemical analysis shows the expression of transcription factors like BRN3A, BRN3B, SNCG, RNA-binding proteins like RBPMS, CD90/THY1 and other cytoskeletal markers like MAP2 and TUJ1; signifying the characteristic of RGCs and RGC expression (FIG. 6A).

Two out of the six cells tested demonstrated action potential firing in the current clamp mode in response to the depolarizing currents. The responses of one of these cells (cell a) are illustrated in FIG. 6B. Although cells are not expected to fire action potentials under ideal voltage-clamp conditions, we believe that imperfect clamping in distal processes can lead to such firing observed as illustrated in FIG. 5C for the same cell a from FIG. 6D. Interestingly, all but one cell (6 out of 6 and 4 out of 5 DIV35 old cells) demonstrated reliable firing when depolarized with voltage steps in the voltage-clamp mode. In FIG. 6B, cell b illustrates firing of the older cells and cell c illustrates firing of the younger cells in response to the depolarizing voltage steps. As expected, later stage iPSC-RGCs cultures (D75) produced higher frequency, sustained firing and generated larger spikes when compared to early born iPSC-RGCs.

Cells firing under current-clamp conditions had resting membrane potentials around −50 mV. In contrast, cells that fired under voltage-clamp but not current-clamp conditions had resting potentials around or above −30 mV. In voltage clamp mode membrane potential was maintained at −60 mV allowing more effective recovery of sodium channels from inactivation after depolarizing steps, thus enabling action potential firing in response to depolarization.

### Discussion of Example 1

We employed a two-step/stage differentiation to induce RGC differentiation from iPSCs. The first stage involved differentiation of iPSCs to RPCs. The RPCs were matured in a stage-specific manner using small molecules and recombinant proteins to modulate SHH pathway, Wnt pathway and Notch signaling to reliably produce abundant RGCs, which stained positive for RGC markers and emulated action potential.

This method is a quick and efficient RGC generation protocol without the need for 3D aggregate formation or manual enrichment to initiate RGC differentiation. In our method, the entire hiPSCs monolayer was differentiated to RPCs using a chemically defined medium in 2D cultures. We employed crosshatching technique to generate clumps of cells that underwent stage-specific differentiation to produce functionally mature RGCs by DIV28. This provided an accelerated timeline considering other published methods to date. Our methodology involved a chemically defined media of RGC differentiation by pairing with our novel RPC generation protocol to produce RGCs. The RPCs generated showed immunoreactivity to RAX and LHX2 in the RPC lines for over 97% of cells in iPSC culture indicating that our protocol committed cells towards RPC lineage.

Using our method, we reliably differentiated six normal iPSC lines and multiple clones from those lines (data not shown) to generate mature RGCs that stain positive for Thy1/Tuj1, BRN3A, BRN3B, TUBB3, RBPMS, MAP2 and SNCG markers. Using different chemical conditions, we generated over 80% of pure iPSC-RGC cultures. Using FACS sorting analysis, we quantified the presence of Thy1/CD90.1 positive RGCs (˜91%), Brn3b positive (˜94%), SNCG positive (˜95%) and RBPMS positive (˜35%) iPSC-RGCs in our differentiated cultures.

In addition to the matured RGCs, we also generated fewer other retinal cell types that express pan-retinal markers and appear to be astrocytes, amacrine and/or bipolar cells. We did not produce RPE and photoreceptor outer or inner segments in our differentiations.

Here, we also show valuable data representing a heterogenous population of RGCs that can be characterized by cell type specific gene expression. This is extremely useful in providing a runway for identification of new surface markers specific to RGC subtypes, allowing for selection using magnetic bead isolation, fluorescent activated cell sorting or immunopanning of iPSC derived RGCs.

Furthermore, the RGCs differentiated using the LSBX condition exhibited electrophysiological function, with the ability to conduct sodium and potassium through voltage-dependent channels and fire action potentials. When comparing action potentials obtained from RGCs at day 35, day 75 and day 110, we observed that RGCs were able to fire continuously producing larger spikes with higher frequency, as they matured and aged in culture when compared to younger cells. Based on the nature and type of physiological responses, several RGC subtypes like ON- OFF- and alpha-RGCs were observed. Therefore, our validated methodology can reliably harness iPSC technology as a renewable source of RPCs to efficiently produce highly enriched populations of RGCs for in vitro studies of glaucoma and potential therapeutic modalities for incurable RGC-related diseases.

The present method is a reproducible and efficient chemically defined in vitro approach for generating unprecedented yields of RPC populations from multiple iPSCs, that are then directed toward the RGC lineage. We differentiated multiple iPSC lines into RGCs in a step-wise manner using small molecules and peptide modulators by inhibiting bone morphogenetic protein, TGF-β, and canonical Wnt pathways. Purified populations of these mature iPSC-RGCs can be used to advantage for in vitro studies of glaucoma and for therapeutic purposes for many RGC-related ocular diseases.

### REFERENCES FOR EXAMPLE 1

1 Janssen, S. F. et al. The vast complexity of primary open angle glaucoma: disease genes, risks, molecular mechanisms and pathobiology. Progress in retinal and eye research 37, 31-67 (2013).
2 Tham, Y. C. et al. Global prevalence of glaucoma and projections of glaucoma burden through 2040: a systematic review and meta-analysis. Ophthalmology 121, 2081-2090, doi:10.1016/j.ophtha.2014.05.013 [doi] (2014).
3 Weinreb, R. N. et al. Primary open-angle glaucoma. Nat Rev Dis Primers 2, 16067, doi:10.1038/nrdp.2016.67 (2016).
4 Kapetanakis, V. V. et al. Global variations and time trends in the prevalence of primary open angle glaucoma (POAG): a systematic review and meta-analysis. Br J Ophthalmol 100, 86-93, doi:10.1136/bjophthalmol-2015-307223 (2016).
5 Quigley, H. A. & Broman, A. T. The number of people with glaucoma worldwide in 2010 and 2020. Br J Ophthalmol 90, 262-267, doi:10.1136/bjo.2005.081224 (2006).
6 Yu, D. Y. et al. Retinal ganglion cells: Energetics, compartmentation, axonal transport, cytoskeletons and vulnerability. Prog Retin Eye Res 36, 217-246, doi:10.1016/j.preteyeres.2013.07.001 (2013).
7 Dahlmann-Noor, A. H., Vijay, S., Limb, G. A. & Khaw, P. T. Strategies for optic nerve rescue and regeneration in glaucoma and other optic neuropathies. Drug Discov Today 15, 287-299, doi:10.1016/j.drudis.2010.02.007 (2010).
8 Danesh-Meyer, H. V. & Levin, L. A. Neuroprotection: extrapolating from neurologic diseases to the eye. Am J Ophthalmol 148, 186-191 e182, doi:10.1016/j.ajo.2009.03.029 (2009).
9 Sluch, V. M. & Zack, D. J. Stem cells, retinal ganglion cells and glaucoma. Dev Ophthalmol 53, 111-121, doi:10.1159/000358409 (2014).
10 Meyer, J. S. et al. Optic vesicle-like structures derived from human pluripotent stem cells facilitate a customized approach to retinal disease treatment. Stem Cells 29, 1206-1218, doi:10.1002/stem.674 (2011).
11 Singh, R. et al. iPS cell modeling of Best disease: insights into the pathophysiology of an inherited macular degeneration. Hum Mol Genet 22, 593-607, doi:10.1093/hmg/dds469 (2013).
12 Takahashi, K. et al. Induction of pluripotent stem cells from adult human fibroblasts by defined factors. Cell 131, 861-872, doi:10.1016/j.cell.2007.11.019 (2007).
13 Takahashi, K. & Yamanaka, S. Induction of pluripotent stem cells from mouse embryonic and adult fibroblast cultures by defined factors. Cell 126, 663-676, doi:10.1016/j.cell.2006.07.024 (2006).
14 Chamling, X., Sluch, V. M. & Zack, D. J. The Potential of Human Stem Cells for the Study and Treatment of Glaucoma. Invest Ophthalmol Vis Sci 57, ORSFil-6, doi:10.1167/iovs.15-18590 (2016).
15 Tang, Z. et al. Progress of stem/progenitor cell-based therapy for retinal degeneration. J Transl Med 15, 99, doi:10.1186/s12967-017-1183-y (2017).
16 Wright, L. S., Phillips, M. J., Pinilla, I., Hei, D. & Gamm, D. M. Induced pluripotent stem cells as custom therapeutics for retinal repair: progress and rationale. Exp Eye Res 123, 161-172, doi:10.1016/j.exer.2013.12.001 (2014).
17 Eiraku, M. et al. Self-organizing optic-cup morphogenesis in three-dimensional culture. Nature 472, 51-56, doi:10.1038/nature09941 (2011).
18 Nakano, T. et al. Self-formation of optic cups and storable stratified neural retina from human ESCs. Cell Stem Cell 10, 771-785, doi:10.1016/j.stem.2012.05.009 (2012).
19 Gill, K. P., Hewitt, A. W., Davidson, K. C., Pebay, A. & Wong, R. C. Methods of Retinal Ganglion Cell Differentiation From Pluripotent Stem Cells. Transl Vis Sci Technol 3, 7, doi:10.1167/tvst.3.3.7 (2014).
20 Chuang, J. H. et al. Expression profiling of cell-intrinsic regulators in the process of differentiation of human iPSCs into retinal lineages. Stem Cell Res Ther 9, 140, doi:10.1186/s13287-018-0848-7 (2018).
21 Marquardt, T. & Gruss, P. Generating neuronal diversity in the retina: one for nearly all. Trends Neurosci 25, 32-38 (2002).
22 Tanaka, T. et al. Generation of retinal ganglion cells with functional axons from human induced pluripotent stem cells. Sci Rep 5, 8344, doi:10.1038/srep08344 (2015).
23 Maekawa, Y. et al. Optimized Culture System to Induce Neurite Outgrowth From Retinal Ganglion Cells in Three-Dimensional Retinal Aggregates Differentiated From Mouse and Human Embryonic Stem Cells. Curr Eye Res 41, 558-568, doi:10.3109/02713683.2015.1038359 (2016).
24 Ohlemacher, S. K. et al. Stepwise Differentiation of Retinal Ganglion Cells from Human Pluripotent Stem Cells Enables Analysis of Glaucomatous Neurodegeneration. Stem Cells 34, 1553-1562, doi:10.1002/stem.2356 (2016).
25 Deng, F. et al. Stage-specific differentiation of iPSCs toward retinal ganglion cell lineage. Mol Vis 22, 536-547 (2016).
26 Teotia, P. et al. Generation of Functional Human Retinal Ganglion Cells with Target Specificity from Pluripotent Stem Cells by Chemically Defined Recapitulation of Developmental Mechanism. Stem Cells 35, 572-585, doi:10.1002/stem.2513 (2017).
27 Lee, J. et al. Defined Conditions for Differentiation of Functional Retinal Ganglion Cells From Human Pluripotent Stem Cells. Invest Ophthalmol Vis Sci 59, 3531-3542, doi:10.1167/iovs.17-23439 (2018).
28 Zhou, L. et al. Differentiation of induced pluripotent stem cells of swine into rod photoreceptors and their integration into the retina. Stem Cells 29, 972-980, doi:10.1002/stem.637 (2011).
29 Cavodeassi, F. et al. Early stages of zebrafish eye formation require the coordinated activity of Wnt11, Fz5, and the Wnt/beta-catenin pathway. Neuron 47, 43-56, doi:10.1016/j.neuron.2005.05.026 (2005).
30 Hirami, Y. et al. Generation of retinal cells from mouse and human induced pluripotent stem cells. Neurosci Lett 458, 126-131, doi:10.1016/j.neulet.2009.04.035 (2009).
31 Yang, Z., Ding, K., Pan, L., Deng, M. & Gan, L. Math5 determines the competence state of retinal ganglion cell progenitors. Dev Biol 264, 240-254 (2003).
32 Sun, Y. et al. A central role for Islet1 in sensory neuron development linking sensory and spinal gene regulatory programs. Nat Neurosci 11, 1283-1293, doi:10.1038/nn.2209 (2008).
33 Lipton, S. A. & Tauck, D. L. Voltage-dependent conductances of solitary ganglion cells dissociated from the rat retina. J Physiol 385, 361-391 (1987).
34 Sullivan, S. K. et al. High-level transgene expression in induced pluripotent stem cell-derived megakaryocytes: correction of Glanzmann thrombasthenia. Blood 123, 753-757, doi:10.1182/blood-2013-10-530725 (2014).
35 Duong, T. T. et al. Use of induced pluripotent stem cell models to probe the pathogenesis of Choroideremia and to develop a potential treatment. Stem Cell Res 27, 140-150, doi:10.1016/j.scr.2018.01.009 (2018).
36 Maguire, J. A., Gagne, A., Mills, J. A., Gadue, P. & French, D. L. Generation of human control iPS cell line CHOPWT9 from healthy adult peripheral blood mononuclear cells. Stem Cell Res 16, 14-16, doi:10.1016/j.scr.2015.11.005 (2016).
37 Lyssenko, N. N. et al. Directional ABCA1-mediated cholesterol efflux and apoB-lipoprotein secretion in the retinal pigment epithelium. J Lipid Res 59, 1927-1939, doi:10.1194/jlr.M087361 (2018).
38 Chavali, V. R. et al. A CTRP5 gene S163R mutation knock-in mouse model for late-onset retinal degeneration. Hum Mol Genet 20, 2000-2014, doi:10.1093/hmg/ddr080 (2011).
39 Chavali, V. R., Sommer, J. R., Petters, R. M. & Ayyagari, R. Identification of a promoter for the human C1Q-tumor necrosis factor-related protein-5 gene associated with late-onset retinal degeneration. Invest Ophthalmol Vis Sci 51, 5499-5507, doi:10.1167/iovs.10-5543 (2010).
40 Sluch, V. M. et al. Differentiation of human ESCs to retinal ganglion cells using a CRISPR engineered reporter cell line. Sci Rep 5, 16595, doi:10.1038/srep16595 (2015).
41 Lamba, D. A. et al. Generation, purification and transplantation of photoreceptors derived from human induced pluripotent stem cells. PLoS One 5, e8763, doi:10.1371/journal.pone.0008763 (2010).
42 Fligor, C. M. et al. Three-Dimensional Retinal Organoids Facilitate the Investigation of Retinal Ganglion Cell Development, Organization and Neurite Outgrowth from Human Pluripotent Stem Cells. Sci Rep 8, 14520, doi:10.1038/s41598-018-32871-8 (2018).
43 Langer, K. B. et al. Retinal Ganglion Cell Diversity and Subtype Specification from Human Pluripotent Stem Cells. Stem Cell Reports 10, 1282-1293, doi:10.1016/j.stemcr.2018.02.010 (2018).
44 Sluch, V. M. et al. Enhanced Stem Cell Differentiation and Immunopurification of Genome Engineered Human Retinal Ganglion Cells. Stem Cells Transl Med 6, 1972-1986, doi:10.1002/sctm.17-0059 (2017).
45 Meng, Y. et al. Nicotinamide Promotes Cell Survival and Differentiation as Kinase Inhibitor in Human Pluripotent Stem Cells. Stem Cell Reports 11, 1347-1356, doi:10.1016/j.stemcr.2018.10.023 (2018).
46 Buchholz, D. E. et al. Rapid and efficient directed differentiation of human pluripotent stem cells into retinal pigmented epithelium. Stem Cells Transl Med 2, 384-393, doi:10.5966/sctm.2012-0163 (2013).
47 Gallardo, V. & Bovolenta, P. Positive and negative regulation of Shh signalling in vertebrate retinal development. F1000Res 7, doi:10.12688/f1000research.16190.1 (2018).
48 Espuny-Camacho, I. et al. Pyramidal neurons derived from human pluripotent stem cells integrate efficiently into mouse brain circuits in vivo. Neuron 77, 440-456, doi:10.1016/j.neuron.2012.12.011 (2013).
49 Proenca, C. C., Song, M. & Lee, F. S. Differential effects of BDNF and neurotrophin 4 (NT4) on endocytic sorting of TrkB receptors. J Neurochem 138, 397-406, doi:10.1111/jnc.13676 (2016).
50 Jiang, Y. et al. Transcription factors SOX4 and SOX11 function redundantly to regulate the development of mouse retinal ganglion cells. J Biol Chem 288, 18429-18438, doi:10.1074/jbc.M113.478503 (2013).
51 Bergsland, M., Werme, M., Malewicz, M., Perlmann, T. & Muhr, J. The establishment of neuronal properties is controlled by Sox4 and Sox11. Genes Dev 20, 3475-3486, doi:10.1101/gad.403406 (2006).
52 Wen, W., Pillai-Kastoori, L., Wilson, S. G. & Morris, A. C. Sox4 regulates choroid fissure closure by limiting Hedgehog signaling during ocular morphogenesis. Dev Biol 399, 139-153, doi:10.1016/j.ydbio.2014.12.026 (2015).
53 Pillai-Kastoori, L. et al. Sox11 is required to maintain proper levels of Hedgehog signaling during vertebrate ocular morphogenesis. PLoS Genet 10, e1004491, doi:10.1371/journal.pgen.1004491 (2014).
54 De la Huerta, I., Kim, I. J., Voinescu, P. E. & Sanes, J. R. Direction-selective retinal ganglion cells arise from molecularly specified multipotential progenitors. Proc Natl Acad Sci USA 109, 17663-17668, doi:10.1073/pnas.1215806109 (2012).
55 Kay, J. N. et al. Retinal ganglion cells with distinct directional preferences differ in molecular identity, structure, and central projections. J Neurosci 31, 7753-7762, doi:10.1523/JNEUROSCI.0907-11.2011 (2011).
56 Huberman, A. D. et al. Architecture and activity-mediated refinement of axonal projections from a mosaic of genetically identified retinal ganglion cells. Neuron 59, 425-438, doi:10.1016/j.neuron.2008.07.018 (2008).
57 Yonehara, K. et al. Expression of SPIG1 reveals development of a retinal ganglion cell subtype projecting to the medial terminal nucleus in the mouse. PLoS One 3, e1533, doi:10.1371/journal.pone.0001533 (2008).
58 Trakhtenberg, E. F. et al. Cell types differ in global coordination of splicing and proportion of highly expressed genes. Sci Rep 6, 32249, doi:10.1038/srep32249 (2016).
59 Meyer, J. S. et al. Modeling early retinal development with human embryonic and induced pluripotent stem cells. Proc Natl Acad Sci USA 106, 16698-16703, doi:10.1073/pnas.0905245106 (2009).
60 Riazifar, H., Jia, Y., Chen, J., Lynch, G. & Huang, T. Chemically induced specification of retinal ganglion cells from human embryonic and induced pluripotent stem cells. Stem Cells Transl Med 3, 424-432, doi:10.5966/sctm.2013-0147 (2014).
61 Zaghloul, N. A., Yan, B. & Moody, S. A. Step-wise specification of retinal stem cells during normal embryogenesis. Biol Cell 97, 321-337, doi:10.1042/BC20040521 (2005).
62 Klassen, H., Sakaguchi, D. S. & Young, M. J. Stem cells and retinal repair. Prog Retin Eye Res 23, 149-181, doi:10.1016/j.preteyeres.2004.01.002 (2004).
63 Nedergaard, M., Ransom, B. & Goldman, S. A. New roles for astrocytes: redefining the functional architecture of the brain. Trends Neurosci 26, 523-530, doi:10.1016/j.tins.2003.08.008 (2003).
64 Rodriguez, A. R., de Sevilla Müller, L. P. & Brecha, N. C. The RNA binding protein RBPMS is a selective marker of ganglion cells in the mammalian retina. J Comp Neurol 522, 1411-1443, doi:10.1002/cne.23521 (2014).
65 Chavali V R M, Haider N, Rathi S, et al. Dual SMAD inhibition and Wnt inhibition enable efficient and reproducible differentiations of induced pluripotent stem cells into retinal ganglion cells. Sci Rep. 2020; 10(1):11828. Published 2020 Jul. 16. doi:10.1038/s41598-O₂₀-68811-8

### Example 2

### Method for Production of Recombinant RGCS

Here, we compared the effects of SIRT1 overexpression in experimental optic nerve crush (ONC) experiments via AAV gene transfer to RGCs. We developed and characterized AAV7m8 vectors that drive RGC-specific expression of human SIRT1 in vitro in iPSC-RGCs and in the mouse retina. This vector can be used to drive RGC-specific expression of any gene of interest in a natural RGC or a RGC produced through the methods provided herein. In certain embodiments, a gene of interest is any gene that is neuroprotective of RGC, preserves vision and/or suppress RGC death. We examined the neuroprotective contribution of SIRT1 gene augmentation in suppressing RGC death and vision loss in mice that have undergone ONC using iPSC-RGCs.

### Methods and Materials for Example 2

### Animals

C57Bl/6J mice were obtained from the Jackson Laboratory and raised in a 12-h light/dark cycle. Animals were housed at the University of Pennsylvania in compliance with ARVO Statement for the Use of Animals in Ophthalmic and Vision Research as well as with institutional and federal regulations.

### Cell Culture

The human iPSCs were generated from keratinocytes or blood cells via polycistronic lentiviral transduction (Human STEMCCA Cre-Excisable constitutive polycistronic [OKS/Myc] Lentivirus Reprogramming Kit, Millipore) by University of Pennsylvania iPSC Core facility and characterized with a hES/iPS cell pluripotency RT-PCR kit [11]. The induced pluripotent stem cell-derived retinal ganglion cells (iPSC-RGCs) were derived using the protocol described above. The iPSC-RGCs cells with structural and functional features characteristic of native RGCs are used herein.

We seeded iPSC-RGCs cells at a density of 350,000 cells and transduced with AAV2 vectors at a multiplicity of infection (MOI) of 100,000 vector genomes (vg) per cell. iPSC-RGCs were harvested 48 h post-transduction for immunocytochemistry analysis. Cells were rinsed with 1×PBS and fixed in 4% paraformaldehyde (PFA) for 15 min at room temperature. Afterwards, cells were blocked in 0.1% Triton X-100 and 1% bovine serum albumin (BSA) for 30 min at room temperature. Cells were incubated with primary antibody solution in 1% BSA and rabbit anti-FLAG antibody (CST #14793; 1:200), SIRT1 antibody (sc-74465; 1:200), or BRN3A (EP1972Y, 1:2000) for 1 h at room temperature. Cells were washed with 1×PBS and incubated in secondary antibody solution containing 1% BSA and goat anti-rabbit AlexaFluor-594 antibodies (1:500) for 1 h at room temperature. Cells were removed from secondary incubation, washed in 1×PBS, and mounted with (Fluoromount-G; Southern Biotech; Birmingham, Ala., USA) containing DAPI.

### AAV Vector Design and Production

Human SIRT1 (transcript variant 1) cDNA clones were obtained from Origene. Sequences were amplified with Q5 DNA polymerase (NEB) and cloned into an AAV expression plasmid using a commercial cloning kit (In-Fusion HD; Clontech Laboratories, Mountain View, Calif., USA). Transgene expression was driven by either the CAG promoter derived from pDRIVE-CAG (InvivoGen, San Diego, Calif., USA) [13] or the codon optimized SNCG (gamma-synuclein promoter) [13]. Both cDNA sequences contained a C-terminal 3×FLAG epitope tag that terminates into a bovine growth hormone polyadenylation sequence. AAV expression cassettes were flanked by the AAV2 inverted terminal repeats. A pro-viral plasmid driving expression of enhanced green fluorescent protein (eGFP) was used [8] and contains identical cis regulatory elements. AAV2-CAG.SIRT1, AAV2-CAG.eGFP, AAV7.m8-SNCG.SIRT1, AAV7m8-SNCG.eGFP vectors were generated using previously described methods and purified with CsCl gradient by the CAROT research vector core at the University of Pennsylvania [14]. The AAV7m8 capsid plasmid was a kind gift from Dr. John Flannery (UC-Berkeley). (Dalkara D, Byrne L C, Klimczak R R, et al. In vivo-directed evolution of a new adeno-associated virus for therapeutic outer retinal gene delivery from the vitreous. Sci Transl Med. 2013; 5(189):189ra76. doi:10.1126/scitranslmed.3005708)

### Intravitreal Injections

4-week-old mice were anesthetized by isoflurane inhalation. A 33½ gauge needle was used to create a small incision at the limbus. Afterward, a 10-uL Hamilton syringe (701 RN; Hamilton Company, Reno, Nev., USA) attached to a 33-gauge blunt-end needle was inserted into the vitreous cavity with the needle tip placed directly above the optic nerve head. Two μL of AAV preparation containing approximately 1×10¹⁰ vector genomes was dispensed into each eye bilaterally. Vehicle treated eyes were injected with an equivalent volume of vector dilution buffer (0.001% Pluronic F68 in PBS). The two eyes of each mouse received different injections (vehicle, AAV2-CAG.SIRT1, AAV7m8-SNCG.SIRT1, AAV2-CAG.eGFP, or AAV7m8-ANCG.eGFP) allowing each eye to serve as an independent experimental end point.

### Optic Nerve Crush

Optic nerve crush (ONC) was performed on 12-week-old C57Bl/6J wild-type mice as in our prior studies [4]. Mice were anesthetized systemically with xylazine and ketamine and topically with 0.5% proparacaine eye drops. Under a dissecting microscope, the conjunctiva was lifted with fine forceps and cut using scissors, exposing the sclera. Forceps were used to manipulate and retract orbital fat and muscles, allowing for exposure of the optic nerve. Injury to the optic nerve was induced using curved fine-tip forceps to induce a focal crush injury to the optic nerve ˜1 to 2 mm behind the globe. Maximal pressure was used to close the forceps for 1 s. Use of fine-tip forceps facilitated avoidance of orbital vessels to avoid ocular ischemia. Bleeding during the procedure was categorized as none, minimal, moderate, and large. Mice were excluded for more than moderate bleeding. In each experiment, the ONC was performed in one eye only, allowing the contralateral uninjured eye to serve as a control. The surgeon performing ONC was masked to eyes that received the AAV constructs.

### Optokinetic Response Recordings (OKRs)

Visual function was assessed by measuring the OKR using commercial software and apparatus (OptoMetry; CerebralMechanics, Inc., Medicine Hat, AB, Canada) as previously described [15]. OKR was determined as the highest spatial frequency where mice track a 100% contrast grating that is projected at different spatial frequencies. Measurements were performed by an investigator masked to the experimental treatments. Each datapoint represents ten animals in experiments performed in triplicate.

### Retinal Histology and RGC Quantification

Eyes were harvested and placed in 4% PFA overnight at 48° C. Eyes were washed in PBS followed by dissection of retinal cups. Tissues were permeabilized and blocked in 2% Triton X-100, 10% normal bovine serum, and PBS and then incubated with goat anti-Brn3a antibody (Novus Biologics) diluted 1:100 at 48° C. Retinal cups were washed and then incubated in secondary antibody solution containing 2% Triton X-100, 10% normal bovine serum, and donkey anti-goat AlexaFluor 594 antibody (1:500 dilution). After washing, samples were prepared as flat mounts and mounted onto glass slides with an aqueous mounting medium (SouthernBiotech) containing 4′,6-diamidino-2-phenylindole (DAPI). RGCs were quantified as previously described [5, 6, 16, 17]. Briefly, retinal micrographs were recorded at ×40 magnification in 12 standard fields (from the center of the retina in each quadrant). Total RGC counts from the 12 fields per retinal sample covering a total area of 0.45 mm²/retina were recorded by an investigator masked to the experimental conditions using Photometrics cell counting software. Retinal cross-sections were incubated in blocking buffer containing PBS, 2% Triton X-100, and 10% normal donkey serum for 1 h at room temperature. Next, sections were incubated in primary antibody solution containing the previously described components and a rabbit anti-FLAG antibody (CST #14793) at 1:100 dilutions overnight in a humidified chamber at room temperature. Sections were washed in PBS three times and incubated in secondary antibody solution containing donkey anti-rabbit AlexaFluor 488 antibody diluted at 1:200 for 2 h at room temperature. Slides were then washed in PBS three times and mounted with aqueous mounting medium (SouthernBiotech) containing DAPI. Retinal whole mount photography and counting of RGCs was performed by a masked investigator. Each experiment represents ten animals per group with experiments performed in triplicate.

### Axon Analysis

Neurofilament staining was performed and quantified in longitudinal paraffin embedded sections. Briefly, optic nerves were isolated, processed and embedded in paraffin. 5 μm longitudinal paraffin sections were deparaffinized, rehydrated, and nonspecific binding was reduced using Blocking reagent (Vector Laboratories, Burlingame, Calif., USA). Specimens were then incubated in rabbit anti-neurofilament antibody 1:500 (Abcam, Cambridge, Mass., USA) at 4° C. overnight. Sections were washed three times with PBS, then incubated with anti-rabbit secondary antibody (Vectastain Elite ABC Rabbit kit) for 30 min at 37° C. Avidin/Biotin Complex detection was performed by incubating with Vectastain Elite ABC reagent at 37° C. for 30 min and DAB (diaminobenzidine, Vector labs) substrate for 3 min at RT followed by washing in running water for 5 min. Dehydrated slides were mounted using Refrax mounting medium. Photographs of three fields/nerve (one each at the distal, central, and proximal regions of the longitudinal optic nerve section) at ×40 magnification were taken by a masked investigator. Neurofilament staining optical density was quantified by using ImageJ software (http://nih.gov).

Bisected optic nerves were incubated in 2% osmium tetroxide and dehydrated in graded ethanol immersions. The nerves were then embedded in epoxy resin Embed 812 (Electron Microscopy Sciences, Hatfield, Pa.), 0.75 μm thick cross sections were generated from a section of the nerve 1.5 mm posterior to the globe and stained with 1% toluidine blue. Each optic nerve cross section was analyzed at five standardized photomicrographs (75×75 μm) obtained at ×100 magnification: one in the center and in four quadrants. Axon counts were obtained by a masked operator using the AxonJ ImageAnalysis algorithm plugin for ImageJ (http://imagej.nih.gov/ij/plugins/axonj/) [18]. Mean±standard error of the mean (SEM).

### Statistics

All data are represented as mean±SEM. Each experiment was repeated 3 times for statistical analysis. Differences between treatment groups with respect to OKR responses, RGC quantification, and optic nerve histopathology were compared using a 1-way ANOVA followed by Tukey's honest significant difference test using statistical software (GraphPad Prism 5.0; GraphPad Software, Inc., La Jolla, Calif., USA). Differences were considered statistically significant at P<0.05. Data meet the assumption of normal distribution of tests with variances between GFP and SIRT1 groups. No randomization was performed; however, investigators were masked to disease and therapeutic intervention to quantify endpoints.

### Results of Example 2

### Design and Characterization of AAV7m8 Vectors

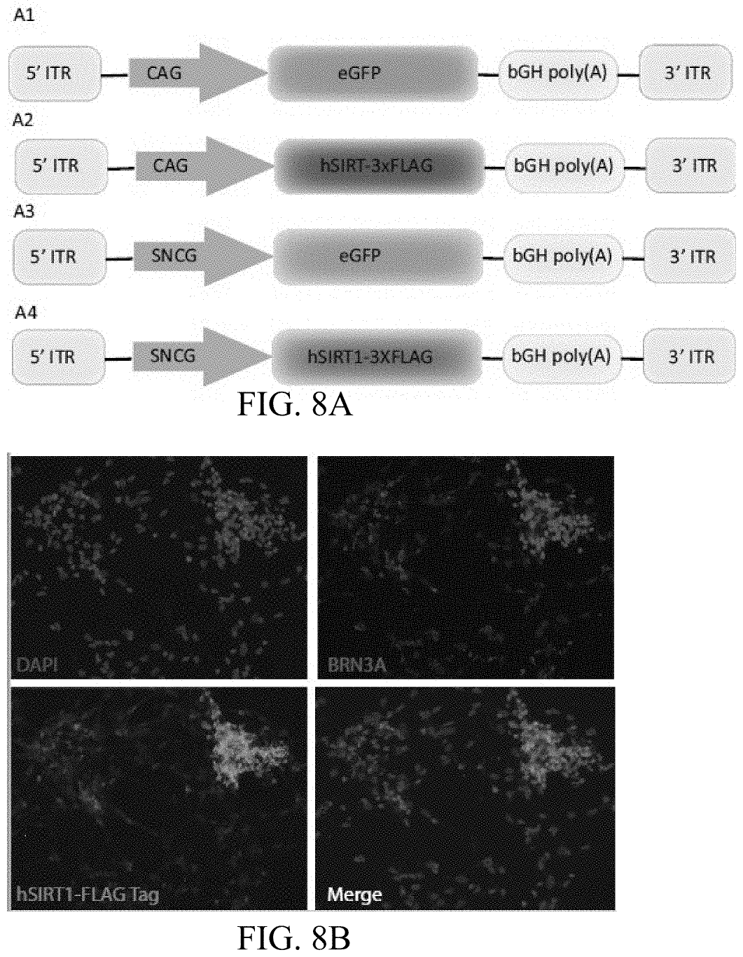

We generated AAV2 and AAV7m8 vectors expressing eGFP and a target gene human SIRT1 driven by the CAG promoter or a RGC-selective gamma synuclein promotor [13] (FIG. 8A). Using induced pluripotent stem cell derived RGCs (IPS-RGCs) developed in a manner previously described and collected from a healthy visually unaffected male, age 32, vector expression was examined in vitro using immunofluorescent labeling on iPSC-RGCs (FIG. 8B). This revealed robust cell number (FIG. 8B) in cultures of viable iPS-RGCs (FIG. 8B), with notable levels of gene expression with cytoplasmic and nuclear distribution of the tagged protein (FIG. 8B).

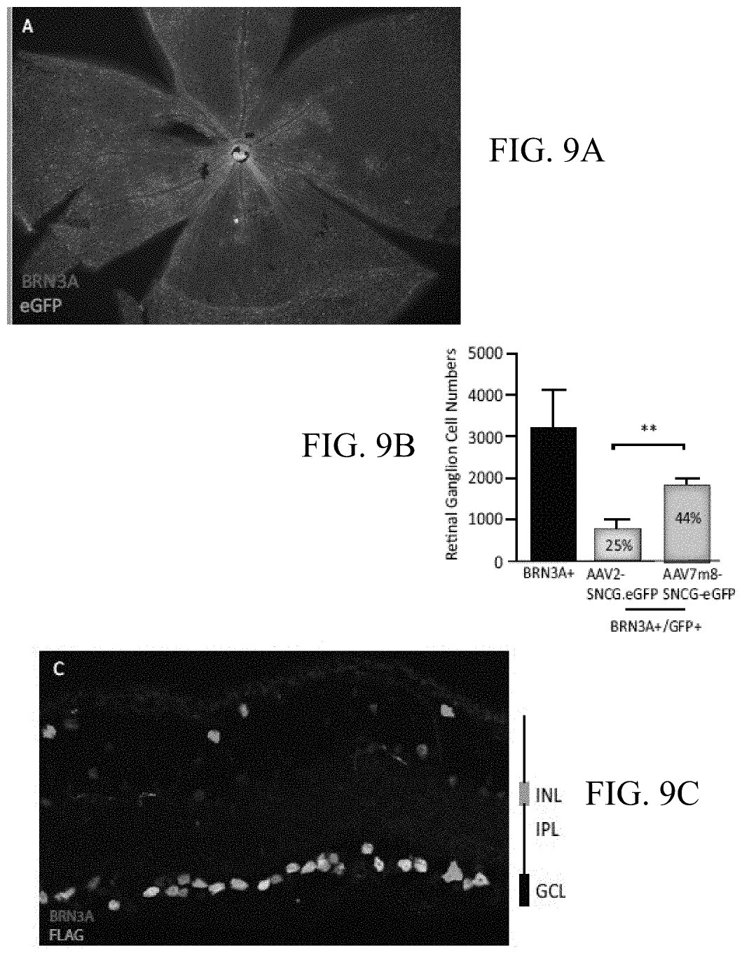

Next, we compared the retinal transduction profile of AAV2-CAG.eGFP to AAV7m8-SNCG.eGFP following intravitreal delivery with a vector expressing enhanced green fluorescent protein in a cohort of wild-type mice as previously described [20]. This vector was compared to a previously described vector developed with a ubiquitous CAG promoter expressing the same human and codon optimized target gene SIRT1 but packaged in an AAV2 capsid. The AAV2.7m8-eGFP vector displayed a higher transduction efficiency of the ganglion cell layer than previously published [8]. Compared with the 25% RGC transduction displayed by the AAV2-CAG.eGFP vector, AAV7m8-SNCG.eGFP achieved ˜44% RGC transduction by quantifying the number of eGFP positive RGCs labeled with BRN3A antibody in retinal whole mounts (FIG. 9A). AAV7m8 vectors driving expression of SIRT1 were injected in to the right and left eyes, respectively, all wild-type mice displayed similar transduction profiles in vivo (FIG. 9B).

Constitutively Expressed SIRT1 Gene Transfer Using AAV2 Vector does not Rescue RGC Protection after ONC

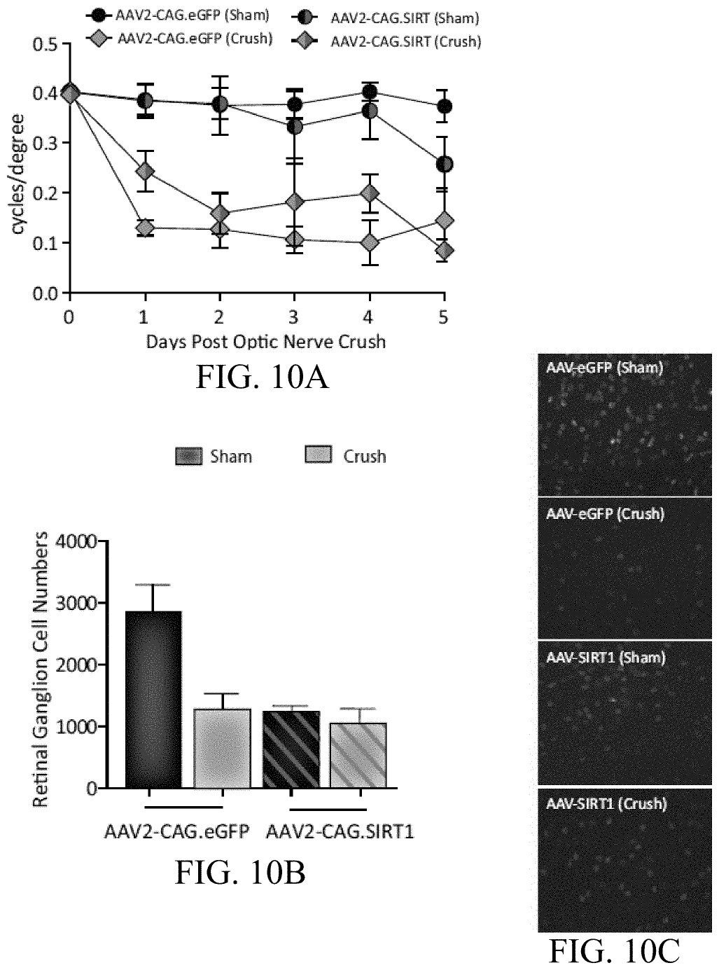

C57B16/J mice received intravitreal injections of experimental or control AAV2 vectors at postnatal week 4 followed by optic nerve crush (ONC) induction at postnatal week 12 (FIG. 10A). Following ONC [4, 21] we measured visual function by OKR daily. Control treated animals treated post-intravitreal injections of AAV2-CAG.eGFP exhibit normal OKR scores (FIG. 10A) and retained RGCs (FIG. 10B, 10C) throughout the experimental timeline (FIG. 10A) which suggests no adverse effects associated with delivery or overexpression of the control transgene. Similarly, animals injected with AAV2-CAG.SIRT1 displayed strong responses prior to induction. Following optic nerve trauma, ONC animals exhibit a decline in OKR scores beginning at day 1 post trauma, which is sustained through the experimental timeline. AAV2-CAG.SIRT1 transfection demonstrated no significant protection of visual acuity throughout the experimental timeline (FIG. 10A).

Permanent visual decline observed in ONC uniformly results in loss of RGC numbers [4, 21]. Retinas from each treatment group were stained with antibodies directed against Brn3a to determine whether SIRT1 augmentation conferred a protective advantage upon RGCs during ONC (FIG. 10C). Intravitreal injection of AAV2 was well tolerated as indicated by comparative total RGC counts in sham-induced animals treated with eGFP. In mice traumatized with ONC, RGC numbers were significantly reduced in all treatment groups compared to sham-induced controls. This demonstrated no significant effect of SIRT1 overexpression using a ubiquitous CAG driven promoter on RGC survival that transfects only 24% of RGCs (FIG. 9).

Ganglion Cell-Selective Expression of SIRT1 Mediated by AAV7m8 Vector with the SNCG Delays Loss of Visual Function and Protects Against RGC Loss after ONC

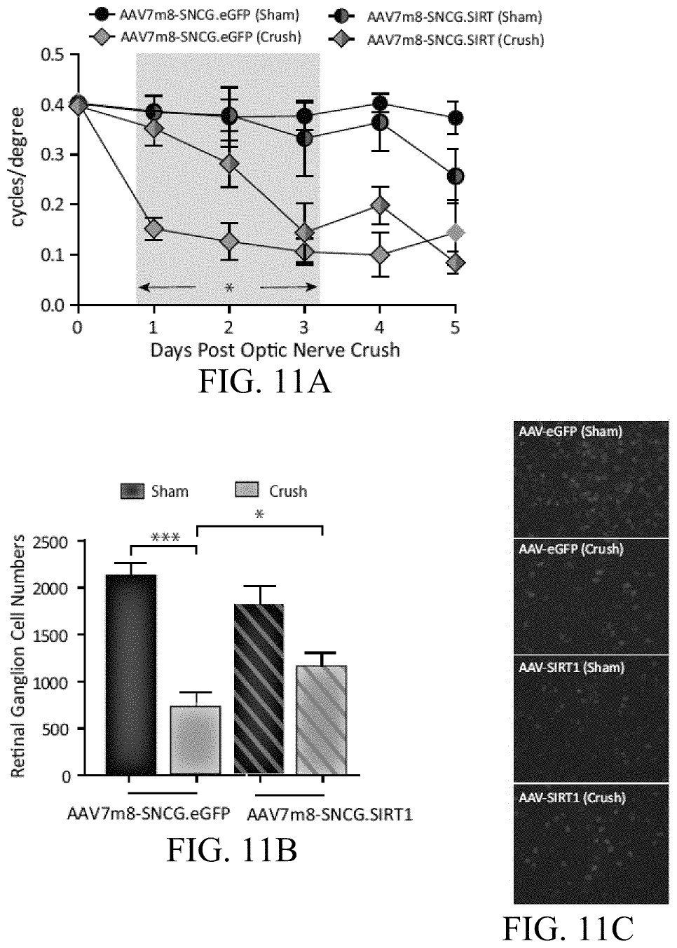

C57B16/J mice received intravitreal injections of AAV7m8 experimental and control vectors at postnatal week 4 followed by ONC at postnatal week 12. Sham Intravitreal injections of AAV2.7m8-eGFP did not interfere with OKR scores prior to ONC (FIG. 11A). Similarly, animals injected with AAV2-SNCG.SIRT1 displayed strong responses prior to induction. Following optic nerve trauma, untreated or AAV7m8-SNCG.eGFP-treated ONC animals exhibit a decline in OKR scores beginning at day 1 post trauma, which is sustained through the experimental timeline. However, eyes treated with AAV7m8-SIRT1 demonstrate a delay in reduction of functional responses by day 2 (FIG. 11A), (AAV2-CAG.SIRT1=0.282±0.015; AAV2-CAG.eGFP=0.127±0.028; P=0.002). This effect was able to delay vision loss for 3 days.

Retinas of each treatment group were stained with antibodies directed against Brn3a to determine whether SIRT1 augmentation conferred a protective advantage upon RGCs during ONC (FIG. 11B). Intravitreal injection of AAV7m8-SNCG was well tolerated as indicated by comparative total RGC counts in control AAV2.eGFP-injected animals. Treatment with AAV7m8-SNCG.SIRT1 resulted in a statistically significant increase in RGC survival compared to control eyes treated with AAV7m8-SNCG.eGFP (FIG. 11B, 11C) by day 6 after ONC (AAV7m8-SNCG.eGFP=711.6±513.52; AAV7m8-SNCG.SIRT1=1159.14±400.1; P=0.0156).

### Discussion of Example 2

Adeno-associated virus (AAV) vectors have become the standard for achieving stable gene transfer with a safe clinical profile, especially when targeted to neurons, when taking into account numerous factors including dose, capsid, cassette, and manufacturing process. AAV2-vectors encoding RPE65 demonstrated a robust safety profile following subretinal delivery in human clinical trials for Leber congenital amaurosis type 2 [22, 23]. Our study compared the neuroprotective effects of RGC specific to a non-cell specific gene transfer in an experimental mouse model of ONC. Our results show that SIRT1 driven by a RGC specific promoter delayed loss of visual acuity and enhanced RGC survival in ONC. Interestingly, even with notable loss of visual function (FIG. 10A) there are still significant RGCs present (FIG. 10B, 10C) suggesting that these cell bodies, although present, might be too damaged to convey visual signals. While using different transgene cassettes, this change, and the cell selectivity, resulted in a greater transduction efficiency as well as a more impressive therapeutic effect (FIG. 11). Results show that SIRT1 overexpression specifically in RGCs plays a significant role in delaying loss of RGC function and reducing RGC death from optic nerve injury. The RGC layer, after intravitreal inject of the construct, should receive the highest concentration of the virus. Current results with the AAV7m8 construct indicate that it is the level expression of SIRT1 in RGCs that is critical and sufficient to promote their survival. These effects are similar to neuroprotective effects promoted by pharmacologic and small molecular modulators of the SIRT1 pathway, as seen in mice treated with resveratrol and ST266 during experimental optic neuritis [21, 24]. Together, these results suggest that modulating SIRT1 can promote RGC survival in multiple forms of optic neuropathy.

Under conditions of oxidative stress, SIRT1 is translocated to the nucleus and modulates activity of protein targets primarily involved in oxidative phosphorylation and mitochondrial biogenesis [25, 26]. This function is induced by activating PGC1-α, a transcription regulator of mitochondrial function and antioxidant metabolism [27]. In addition to these known functions, active SIRT1 deacetylates and inhibits the transcription factor, p53, thereby downregulating apoptotic gene expression and thus improving cell viability [28]. With ONC, we observed a statistically significant visual decline at day 1 post trauma with all AAV treated animals subjected to optic nerve insult, and this effect was significantly delayed in AAV7m8-SNCG.SIRT1 treated animals. The RGC survival found in the current study illustrate a key role of SIRT1 signaling specifically within RGCs in SIRT1-mediated neuroprotection.

Treatment with small molecular activators of the SIRT1 pathway, such as resveratrol, preserves visual function and RGC survival in mouse models of optic neuritis and optic nerve trauma [4, 5, 6, 7, 17, 21, 24]. The current results show that the AAV2.7m8-SIRT1 vector has utility in multiple optic neuropathies such as more chronic injury to the optic nerve, including glaucoma, or acute injury, such as ischemic optic neuropathy. Our data demonstrate that with cell specificity using the SNGC promoter, protection of visual function and RGCs is better achieved, suggesting the importance of cell specificity in the design of potential neuroprotective gene therapies for optic neuropathies.

### REFERENCES FOR EXAMPLE 2

1. Jang S Y. Traumatic optic neuropathy. Korean J Neurotrauma. 2018; 14:1-5.
2. Kumaran A M, Sundar G, Chye L T. Traumatic optic neuropathy: a review. Craniomaxillofac Trauma Reconstr. 2015; 8:31-41.
3. Tang Z, Zhang S, Lee C, Kumar A, Arjunan P, Li Y, et al. An optic nerve crush injury murine model to study retinal ganglion cell survival. J Vis Exp. 2011:2685. https://doi.org/10.3791/2685.
4. Zuo L, Khan R S, Lee V, Dine K, Wu W, Shindler K S. SIRT1 promotes RGC survival and delays loss of function following optic nerve crush. Invest Ophthalmol Vis Sci. 2013; 54:5097-102.
5. Shindler K S, Ventura E, Dutt M, Elliott P, Fitzgerald D C, Rostami A. Oral resveratrol reduces neuronal damage in a model of multiple sclerosis. J Neuroophthalmol. 2010; 30:328-39.
6. Shindler K S, Ventura E, Rex T S, Elliott P, Rostami A. SIRT1 activation confers neuroprotection in experimental optic neuritis. Invest Ophthalmol Vis Sci. 2007; 48:3602-9.
7. Khan R S, Dine K, Wessel H, Brown L, Shindler K S. Effects of varying intranasal treatment regimens in ST266-mediated retinal ganglion cell neuroprotection. J Neuroophthalmol. 2019; 39:191-9.
8. McDougald D S, Dine K E, Zezulin A U, Bennett J, Shindler K S. SIRT1 and NRF2 gene transfer mediate distinct neuroprotective effects upon retinal ganglion cell survival and function in experi-mental optic neuritis. Invest Ophthalmol Vis Sci. 2018; 59:1212-20.
9. Chan L, Mahajan V B, Tsang S H. Genome surgery and gene therapy in retinal disorders. Yale J Biol Med. 2017; 90:523-32.
10. DeBusk A, Moster M L. Gene therapy in optic nerve disease. Curr Opin Ophthalmol. 2018; 29:234-8.
11. Yang W, Liu Y, Slovik K J, Wu J C, Duncan S A, Rader D J, et al. Generation of iPSCs as a pooled culture using magnetic activated cell sorting of newly reprogrammed cells. PLoS ONE. 2015; 10: e0134995.
12. Teotia P, Chopra D A, Dravid S M, Van Hook M J, Qiu F, Morrison J, et al. Generation of functional human retinal ganglion cells with target specificity from pluripotent stem cells by chemically defined recapitulation of developmental mechanism. Stem Cells. 2017; 35:572-85.
13. Chaffiol A, Caplette R, Jaillard C, Brazhnikova E, Desrosiers M, Dubus E, et al. A new promoter allows optogenetic vision restoration with enhanced sensitivity in Macaque retina. Mol Ther. 2017; 25:2546-60.
14. Costello F, Coupland S, Hodge W, Lorello G R, Koroluk J, Pan Y I, et al. Quantifying axonal loss after optic neuritis with optical coherence tomography. Ann Neurol. 2006; 59:963-9.
15. Prusky G T, Alam N M, Beekman S, Douglas R M. Rapid quantifi-cation of adult and developing mouse spatial vision using a virtual optomotor system. Invest Ophthalmol Vis Sci. 2004; 45:4611-6.
16. Quinn T A, Dutt M, Shindler K S. Optic neuritis and retinal ganglion cell loss in a chronic murine model of multiple sclerosis. Front Neurol. 2011; 2:50.
17. Fonseca-Kelly Z, Nassrallah M, Uribe J, Khan R S, Dine K, Dutt M, et al. Resveratrol neuroprotection in a chronic mouse model of multiple sclerosis. Front Neurol. 2012; 3:84.
18. Zarei K, Scheetz T E, Christopher M, Miller K, Hedberg-Buenz A, Tandon A, et al. Automated axon counting in rodent optic nerve sections with AxonJ. Sci Rep. 2016; 6:26559.
19. Chavali V R M, Haider N, Rathi S, Alapati T, He J, Gill K, et al. Dual SMAD inhibition and Wnt inhibition enable efficient and reproducible differentiations of induced pluripotent stem cells into retinal ganglion cells. Sci Rep. 2020; 10:11828.
20. Dudus L, Anand V, Acland G M, Chen S J, Wilson J M, Fisher K J, et al. Persistent transgene product in retina, optic nerve and brain after intraocular injection of rAAV. Vision Res. 1999; 39:2545-53.
21. Grinblat G A, Khan R S, Dine K, Wessel H, Brown L, Shindler K S. RGC neuroprotection following optic nerve trauma mediated by intranasal delivery of amnion cell secretome. Investig Ophthalmol Vis Sci. 2018; 59:2470-7.
22. Bennett J, Wellman J, Marshall K A, McCague S, Ashtari M, DiStefano-Pappas J, et al. Safety and durability of effect of contralateral-eye administration of AAV2 gene therapy in patients with childhood-onset blindness caused by RPE65 mutations: a follow-on phase 1 trial. Lancet. 2016; 388:661-72.
23. Maguire A M, Simonelli F, Pierce E A, Pugh E N Jr., Mingozzi F, Bennicelli J, et al. Safety and efficacy of gene transfer for Leber's congenital amaurosis. N Engl J Med. 2008; 358:2240-8.
24. Khan R S, Dine K, Bauman B, Lorentsen M, Lin L, Brown H, et al. Intranasal delivery of a novel amnion cell secretome prevents neuronal damage and preserves function in a mouse multiple sclerosis model. Sci Rep. 2017; 7:41768.
25. Balaiya S, Abu-Amero K K, Kondkar A A, Chalam K V. Sirtuins expression and their role in retinal diseases. Oxid Med Cell Longev. 2017; 2017:3187594.
26. Balaiya S, Ferguson L R, Chalam K V. Evaluation of sirtuin role in neuroprotection of retinal ganglion cells in hypoxia. Invest Oph-thalmol Vis Sci. 2012; 53:4315-22.
27. Nemoto S, Fergusson M M, Finkel T. SIRT1 functionally interacts with the metabolic regulator and transcriptional coactivator PGC-1 {alpha}. J Biol Chem. 2005; 280:16456-60.
28. Luo J, Nikolaev A Y, Imai S, Chen D, Su F, Shiloh A, et al. Negative control of p53 by Sir2alpha promotes cell survival under stress. Cell. 2001; 107:137-48.
29. Khan R S, Fonseca-Kelly Z, Callinan C, Zuo L, Sachdeva M M, Shindler K S. SIRT1 activating compounds reduce oxidative stress and prevent cell death in neuronal cells. Front Cell Neurosci. 2012; 6:63.
30. Ross A G, McDougald D S, Khan R S, et al. Rescue of retinal ganglion cells in optic nerve injury using cell-selective AAV mediated delivery of SIRT1. Gene Ther. 2021; 28(5):256-264. doi:10.1038/s41434-O₂₁-00219-z.
31. Park J, Lucas A M, Zhang X, et al. Exome-wide evaluation of rare coding variants using electronic health records identifies new gene-phenotype associations. Nat Med. 2021; 27(1):66-72. doi:10.1038/s41591-O₂₀-1133-8

### Example 3

### Evaluation of Rare Coding Variants in RGCS

After interrogating a dataset of 10,900 individuals with WES data in PMBB for carriers of rare loss of function (pLOF) variants, we found a novel rare pLOV variant in PPP1R13L, which is associated with primary open angle glaucoma—a disease of the optic nerve head (ONH) that causes progressive vision loss.

### In Silico Analysis for PPP1R13L Expression in Ocular Tissues

To understand the functional relevance of PPP1R13L in the eye, we evaluated its expression in human ocular tissues using the publicly available Ocular Tissue Database (OTDB; on the world wide web at https://genome.uiowa.edu/otdb/). The OTDB consists of gene expression data for eye tissues from 20 normal human donors, generated using Affymetrix Human Exon 1.0 ST arrays and described as Probe Logarithmic Intensity Error (PLIER) values, where individual gene expression values are normalized with its expression in other tissues.

### Gene Expression in DBA/2J Mouse Ocular Tissues

We assessed the gene expression of PPP1R13L in mouse ocular tissues using the publicly available Glaucoma Discovery Platform (http://glaucomadb.jax.org/glaucoma). This platform provides an interactive way to analyze RNA sequencing data obtained from retinal ganglion cells (RGCs) isolated from retina and optic nerve head of a 9-month-old female D2 mouse, which is an age-dependent model of ocular hypertension/glaucoma, and D2-Gpnmb+ mouse that do not develop high IOP/glaucoma. For transcriptomic studies, four distinct groups were compared based on axonal degeneration and gene expression patterns. The transcriptome of D2 group 1 is identical to the control strain (D2-Gpnmb+), while D2 groups 2-4 exhibit increasing levels of molecular changes relevant to axonal degeneration when compared to control group. We used the Datgan software to assess the differential expression of PPP1R13L in the retina.

### Immunolocalization of PPP1R13L in Human Retina

To study the localization of PPP1R13L protein in different retinal layers of the human eye, we performed immunofluorescence on formalin-fixed paraffin-embedded section (N=3) obtained from normal 68-year old donor's cadaver eyes with a commercially available antibody, anti-PPP1R13L (Cat #51141-1-AP, Proteintech, IL, USA). Antigen retrieval was performed in 1× citrate buffer (Life Technologies) warmed to 95° C. for 30 minutes. Sections were allowed to cool to room temperature and subsequently blocked in 10% normal goat serum with 1% bovine serum albumin in 1×TBS buffer for one hour. The retinal distribution of PPP1R13L protein was visualized by incubating the retinal section with rabbit polyclonal anti-PPP1R13L antibody at 1:300 dilution overnight at 4° C., followed by chicken anti-rabbit IgG conjugated with Alexa Fluor 594 (Life Technologies, A21442). Nuclei were stained with the use of Vectashield DAPI in the mounting media. The images were captured using a Zeiss Imager Z1 fluorescence microscope equipped with AxioVS40 software version 4.8.1.0.

Human iPSC-RGC Cultures

The human iPSCs were generated from keratinocytes or blood cells via polycistronic lentiviral transduction (Human STEMCCA Cre-Excisable constitutive polycistronic [OKS/L-Myc] Lentivirus Reprogramming Kit, Millipore) and characterized with a hES/iPS cell pluripotency RT-PCR kit. The induced pluripotent stem cell-derived retinal ganglion cells (iPSC-RGCs) for our studies were derived using the methods described above in Example 1, which includes incubation with small molecules to inhibit BMP, TGF-beta (SMAD) and Wnt signaling thereby differentiating retinal ganglion cells (RGCs) from iPSCs. The iPSCs were differentiated into pure iPSC-RGCs cells with structural and functional features characteristic of native RGC cells as described herein.

Evaluating Oxidative Stress in iPSC-RGCs

Induced pluripotent stem cell-derived retinal ganglion cells (iPSC-RGCs) were incubated with increasing amounts of H₂O₂ overnight before replacing the cultures with complete media. The cells were collected 24 hours after the H₂O₂ treatment, and levels of PPP1R13L transcripts were assessed using quantitative RT-PCR and gene expression primers, Fwd-5′-TGCCCCAATTCTGGAGTAGG-3′ (SEQ ID NO: 1) and Rev-5′-CGGCACGTGGACACAGATT-3′ (SEQ ID NO: 2) following previously established protocols. Mean expression levels (±standard error of mean) were calculated by analyzing at least three independent samples with replica reactions and presented on an arbitrary scale that represents the expression over the housekeeping gene ACTB. Relative gene expression was quantified using the comparative Ct method. The relative gene expression was compared against no treatment control to obtain normalized gene expression.

### Results for Example 3

PPP1R13L is highly expressed in ocular tissues, with optic nerve and the ONH among the highest (Table 7). Table 7 shows expression of PPP1R13L in human ocular tissues per the Ocular Tissue Database (OTDB) ranked from highest to lowest expression per ocular tissue. Expression values are represented as Probe Logarithmic Intensity Error (PLIER) values, where individual gene expression values are normalized to its expression in other tissues

TABLE 7
Expression of PPP1R13L transcript in human ocular tissues

| | PPP1R13L |
|---|---|
| Eye Tissue | Probe ID: 3865344 |
| Expression | Probe Logarithmic Intensity Error (PLIER) |
| Choroid RPE | 36.0174 |
| Ciliary Body | 34.7373 |
| Cornea | 43.0693 |
| Iris | 29.1438 |
| Lens | 39.9613 |
| Optic Nerve | 46.2664 |
| Optic Nerve Head | 41.1548 |
| Retina | 35.4081 |
| Sclera | 49.7938 |
| Trabecular | 39.1355 |
| Meshwork | |

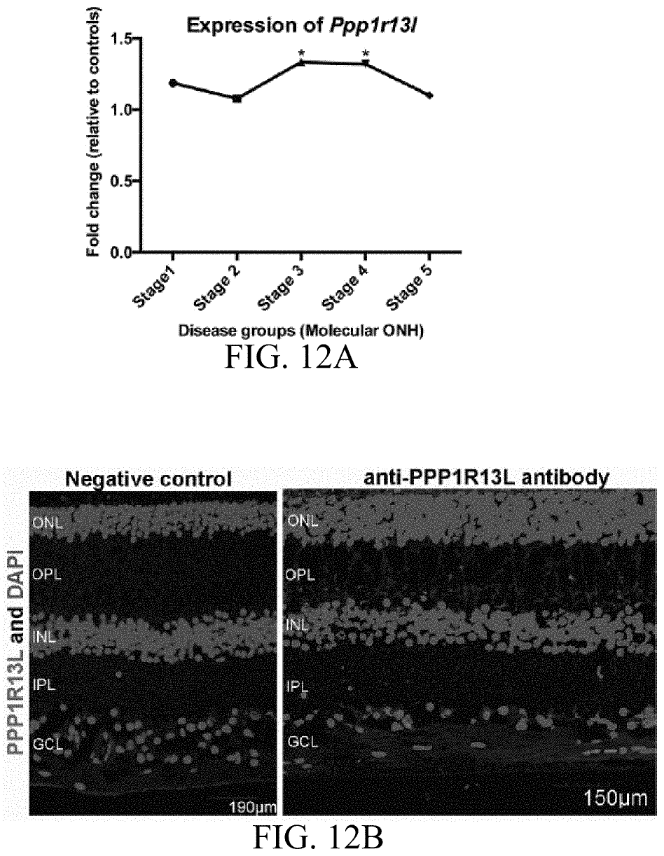

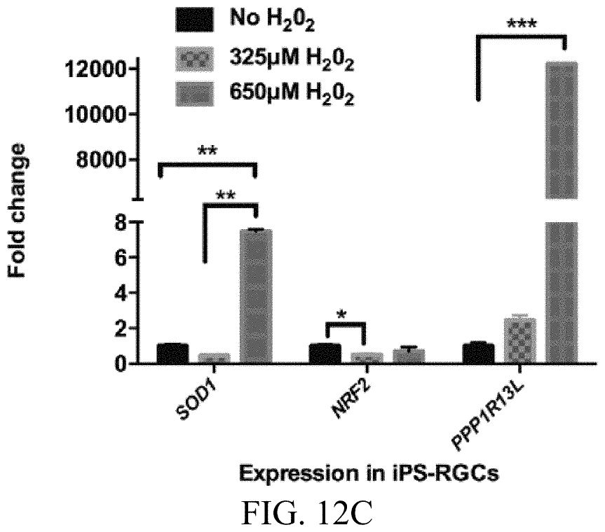

Retinal ganglion cells (RGCs) are the primary cells affected by glaucoma, and cells in the ONH such as astroglia, microglia, and endothelial cells mediate RGC degeneration in response to stress such as increased intraocular pressure. We investigated whether PPP1R13L is differentially expressed in the mouse ONH in glaucoma by comparing microarray gene expression datasets of the ONH. We found PPP1R13L expression to be highest during late-early to moderate stages of glaucoma (FIG. 12A). Additionally, inhibition of PPP1R13L has been shown to exacerbate retinal ganglion cell (RGC) death following axonal injury. We found that the PPP1R13L protein is predominantly localized to the ganglion cell layer in the adult human retina with some expression in the outer and inner plexiform layers, confirming its role in RGC function (FIG. 12B). Using human induced pluripotent stem cell-derived RGCs (iPSC-RGCs), we found that oxidative stress markedly upregulated PPP1R13L expression (FIG. 12C) to a much greater extent than even superoxide dismutase 1 (SOD1), which is known to be transcriptionally upregulated in response to oxidative stress. Thus, PPP1R13L is expressed in RGCs, is significantly upregulated by oxidative stress, and may help to prevent RGC death from p53 activation and p53-mediated apoptosis in primary open angle glaucoma. Haploinsufficiency of PPP1R13L in RGCs increases the visual consequences of primary open angle glaucoma.

### Example 4

### Identification of RGC Specific Markers

Mature iPSC-RGCs at Day 35-37 express RGC specific markers. iPSC-RGCs stain positive and can be segregated and sorted using MACS sorting using CD90.2 antibody. They express RGC specific markers, including Brn3b (82-89%), RBPMS (33-35%), SNCG (84-95%) and CD90 (88-91%). The percentage of cells expressing these markers are the numbers/percentages of cells expressing these markers when iPSCs are differentiated into iPSC-RGCs (with MACS sorting or any physical separation methods).

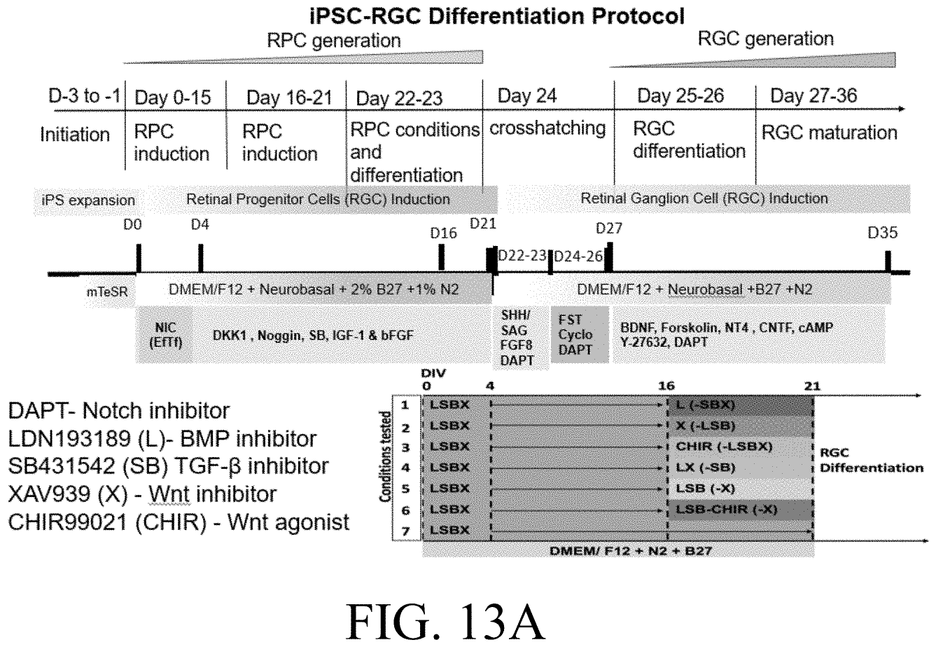

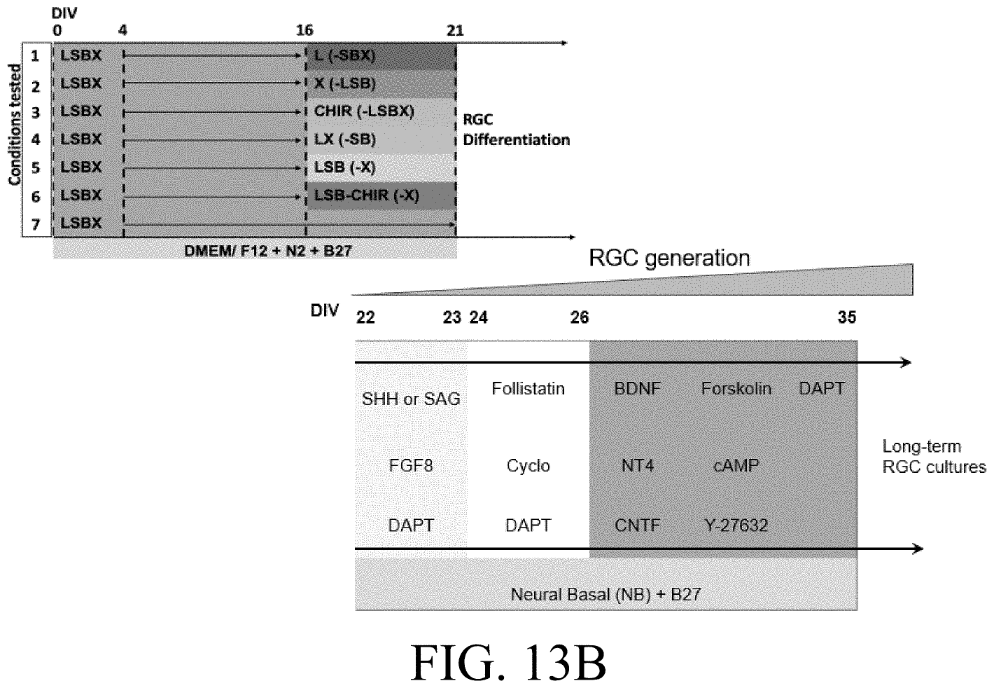

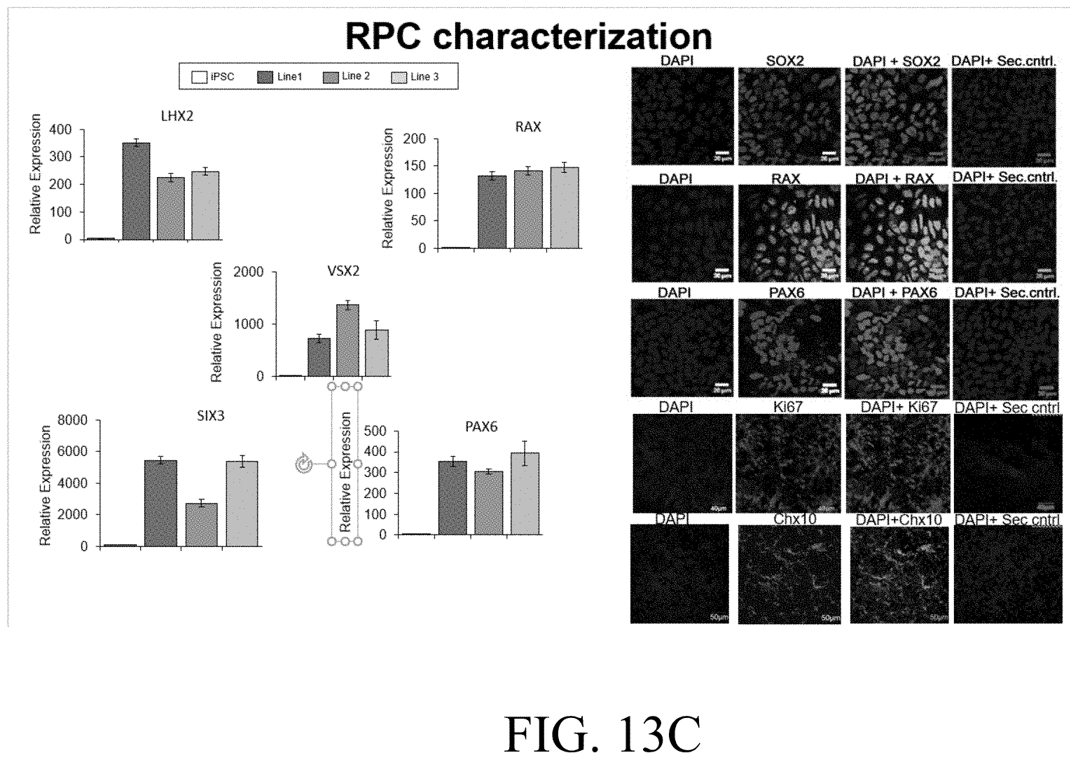

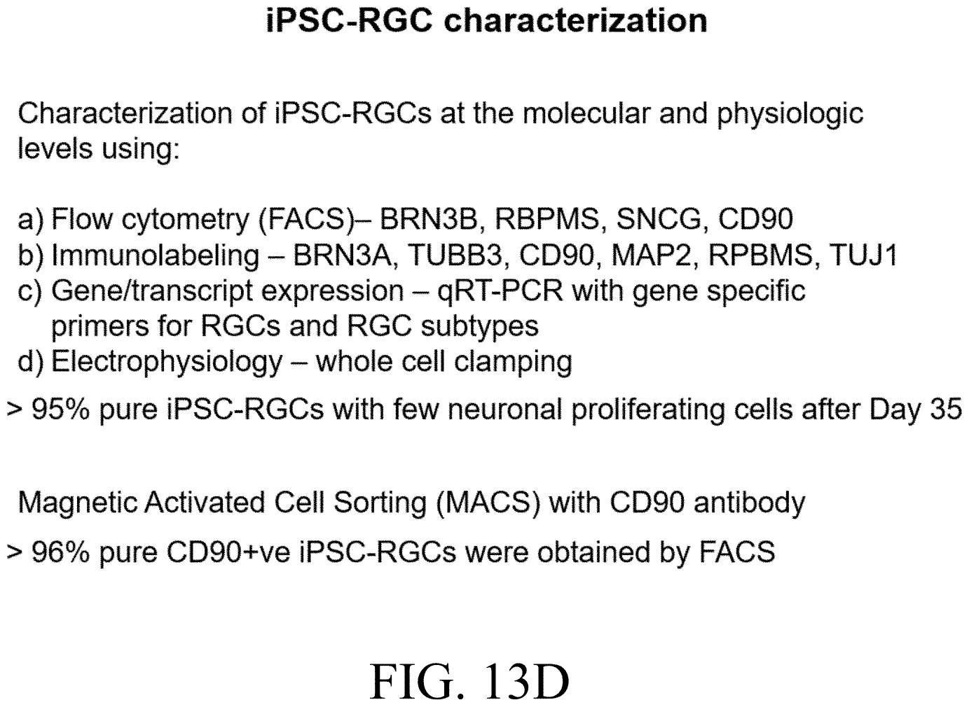

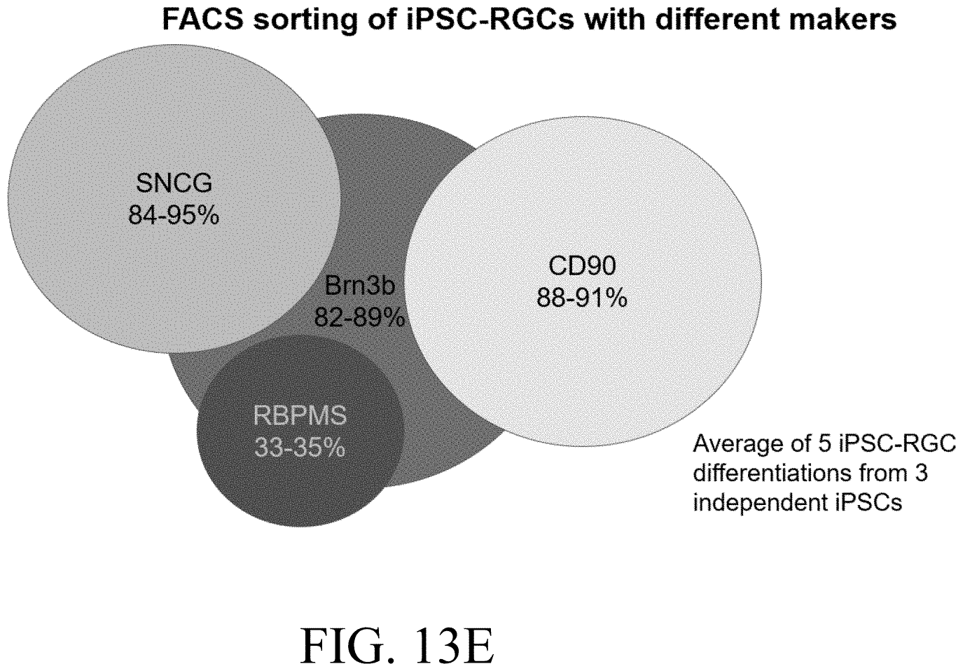

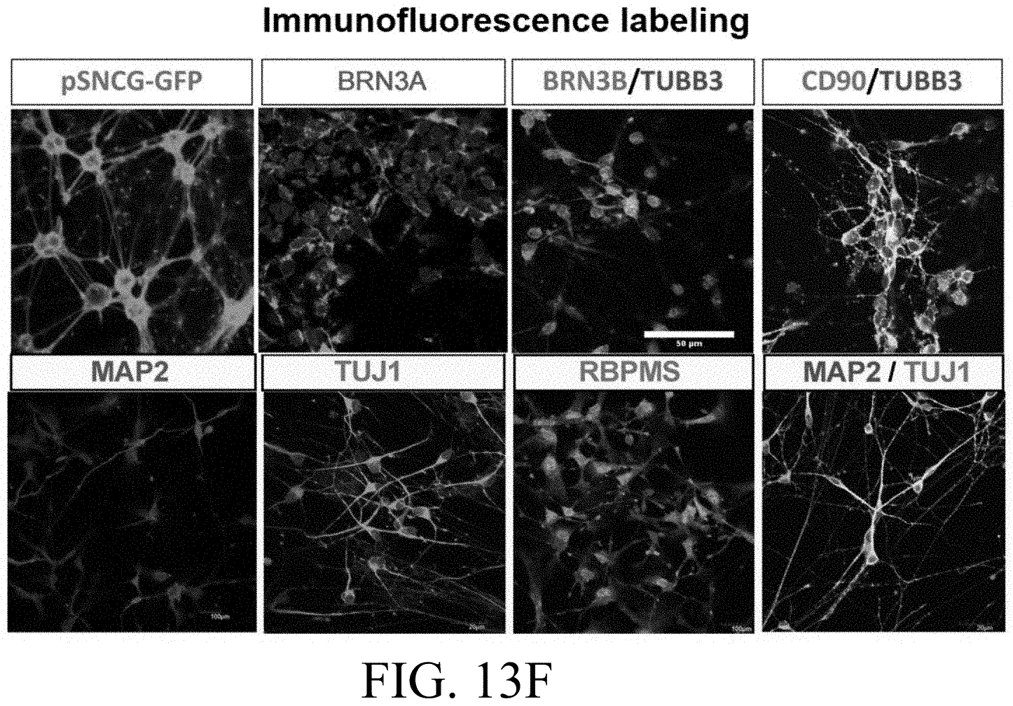

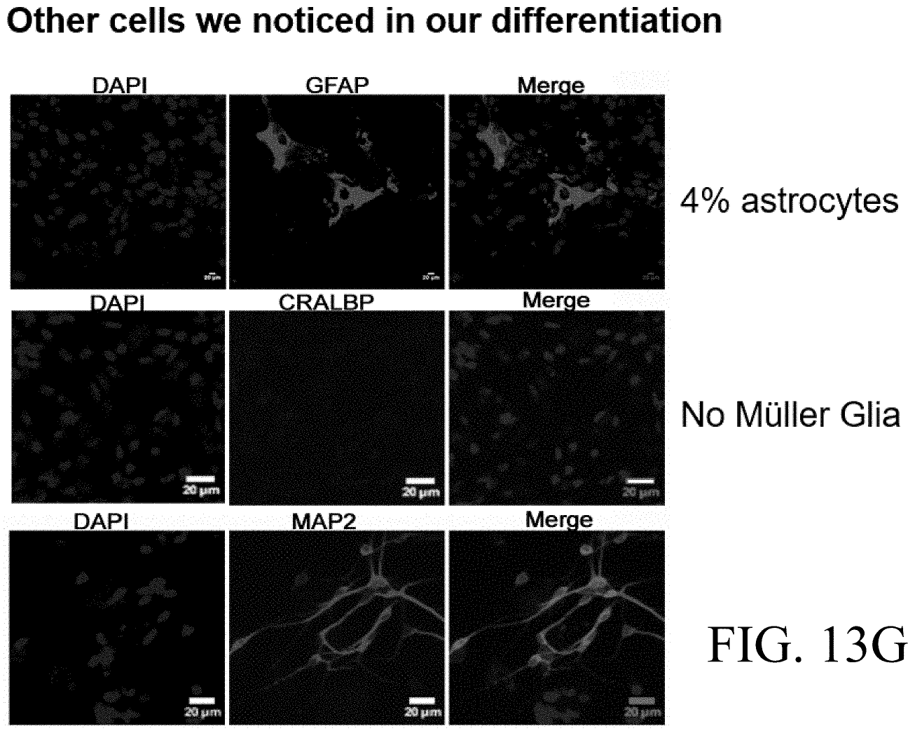

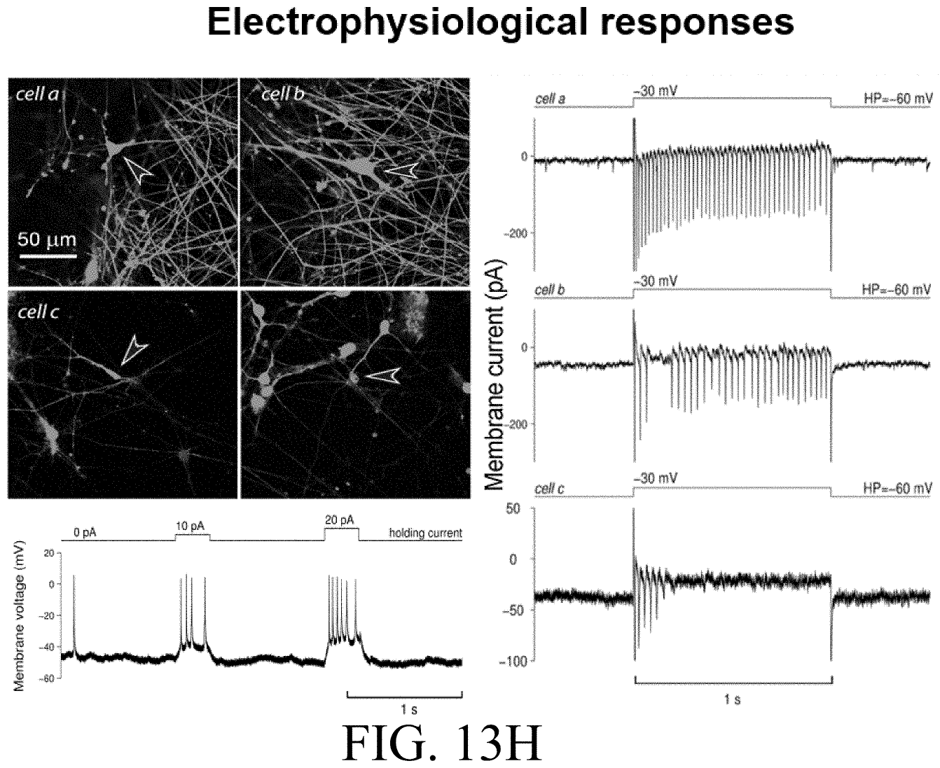

Mature iPSC-RGCs were differentiated according to Schematic described in FIGS. 13A and 13B using a two-step differentiation approach initially to RPCs and then into iPSC-RGCs. RPCs were characterized using qRT-PCR using gene specific primers and ICC using RPC specific antibodies FIG. 13C. Additional immunocytochemistry analysis (FIG. 4) using RGC specific antibodies and purification with MACS with CD90.1 antibody yielded over 96% pure iPSC-RGCs (FIGS. 4, 13D, and 13E).

FIG. 13E shows the results of FACS sorting and provides the percentage of RGC specific markers expressed by iPSC-RGCs from our protocol. FIG. 13E. FIGS. 13F and 13G show RGC characterization using ICC markers and other neuronal cells we obtained using our method. Electrophysiological responses using patch clamp method demonstrates that the mature iPSC-RGCs respond to light stimuli and displays characteristic Ganglion cell responses FIG. 13H.

### Example 5

### In Vivo Application of RGCs in a Mouse Model

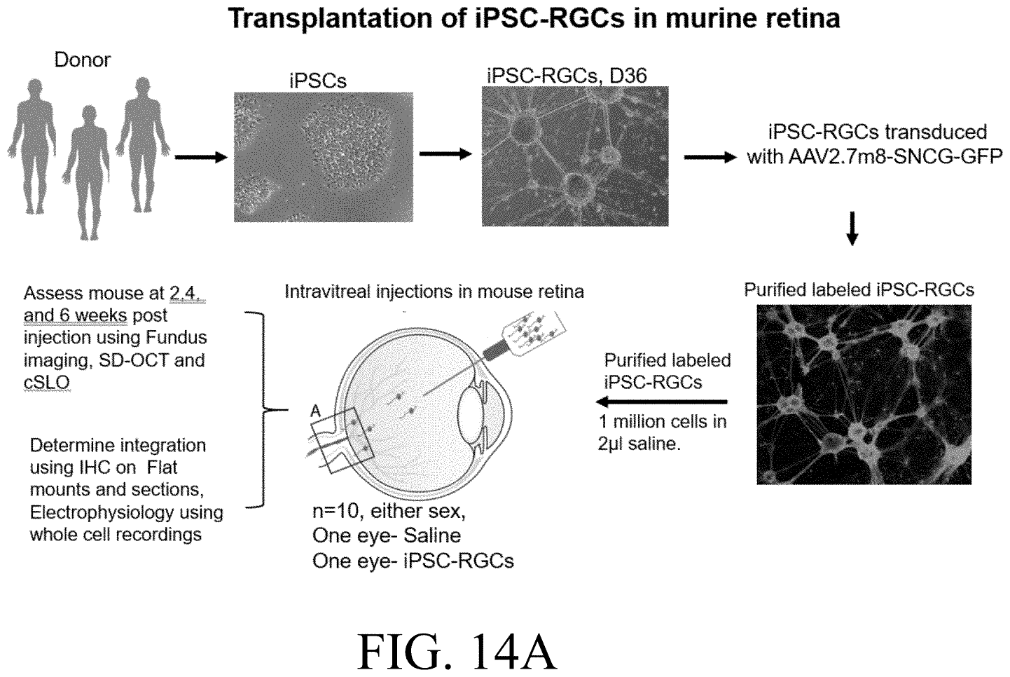

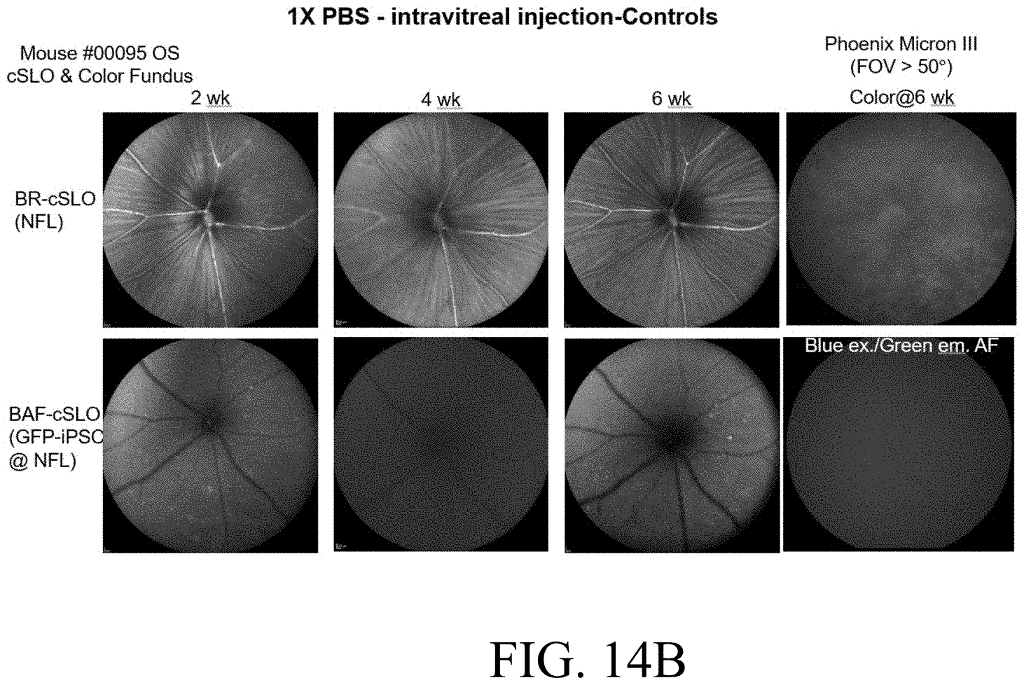

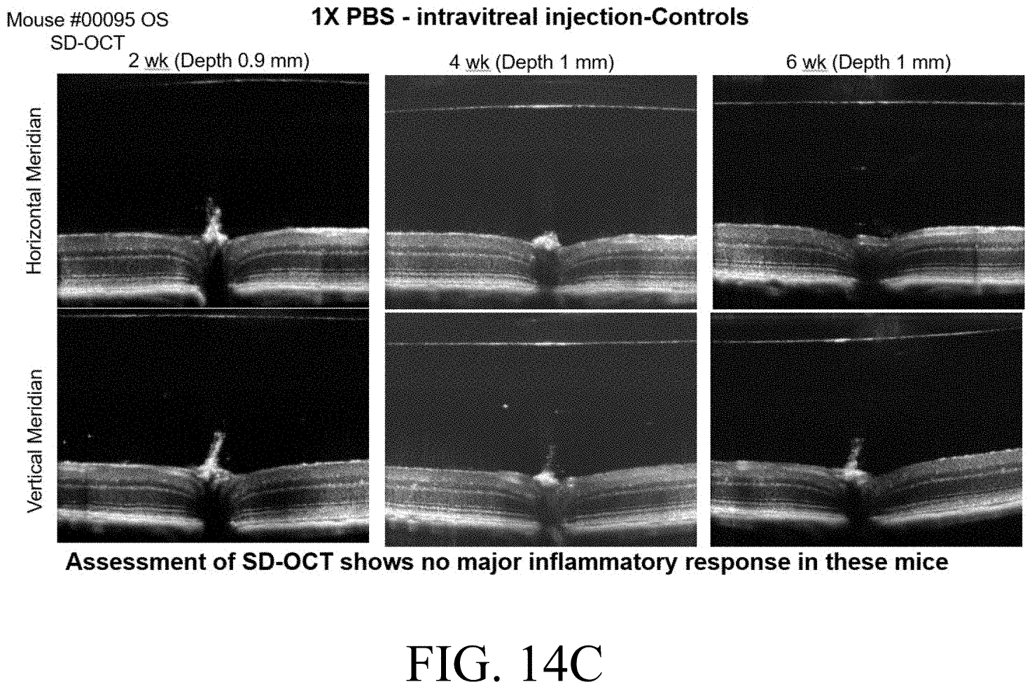

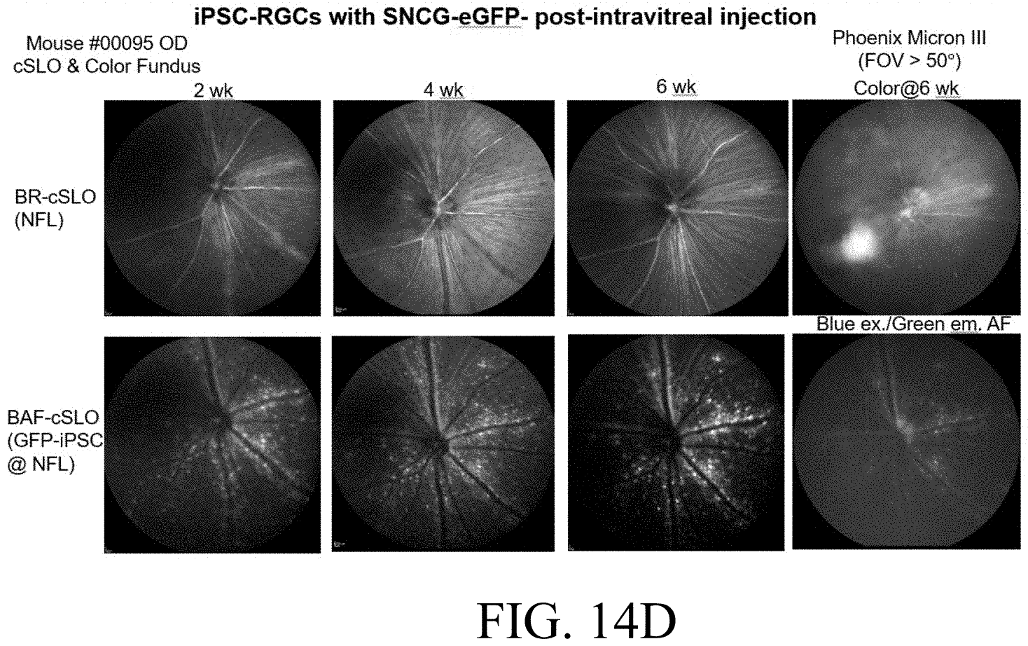

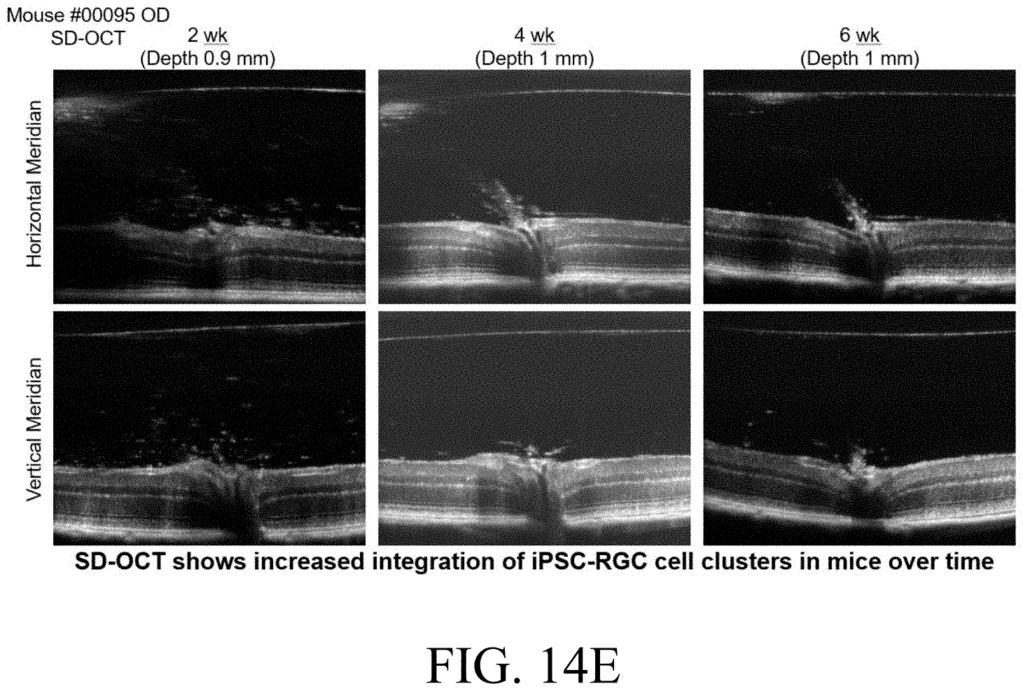

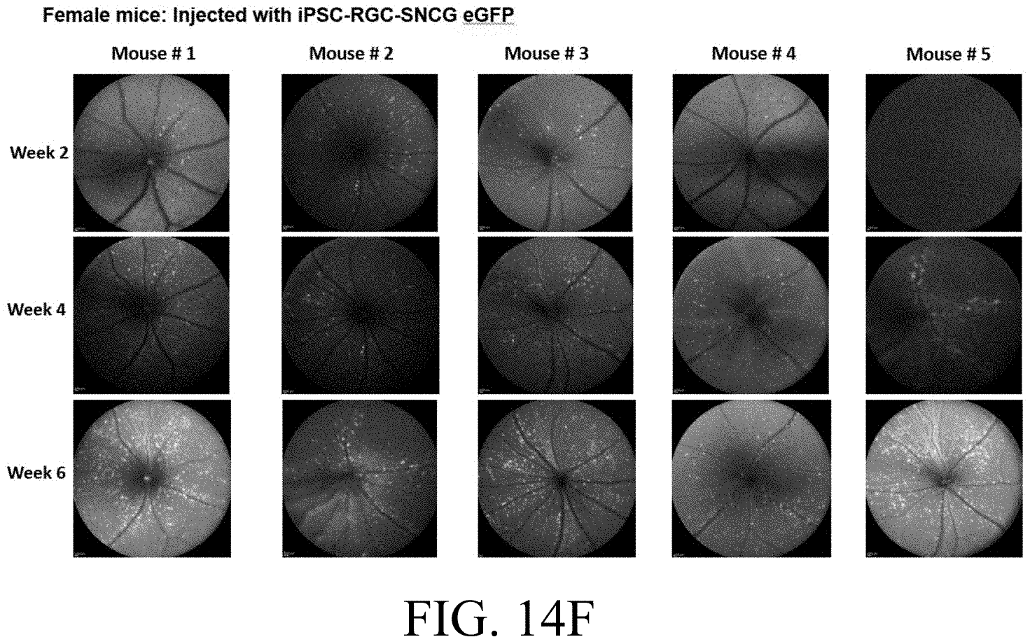

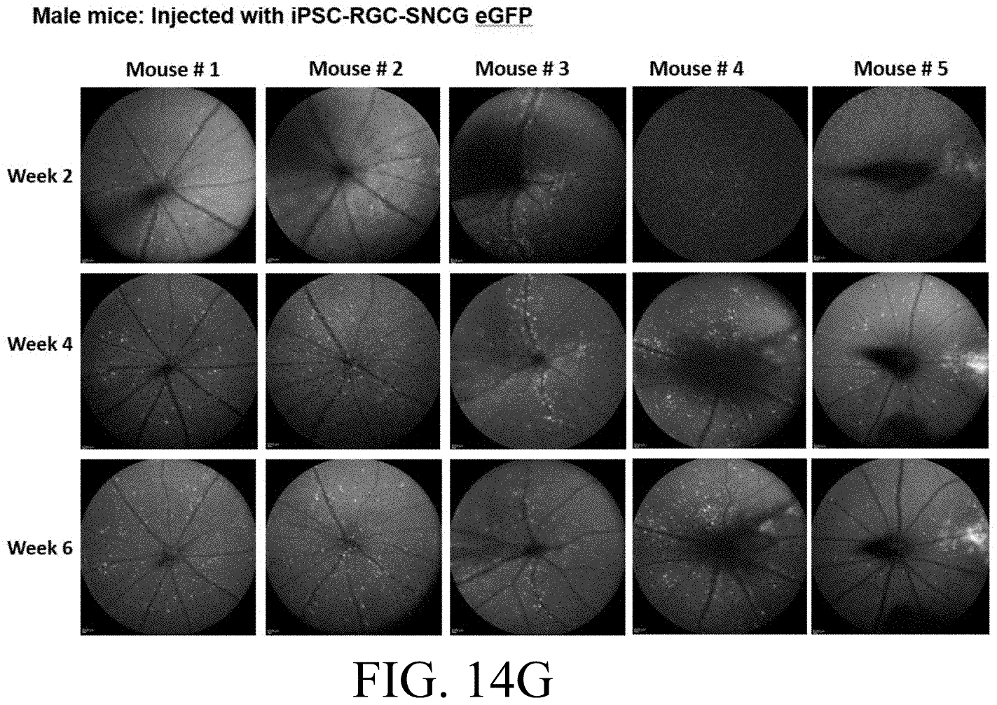

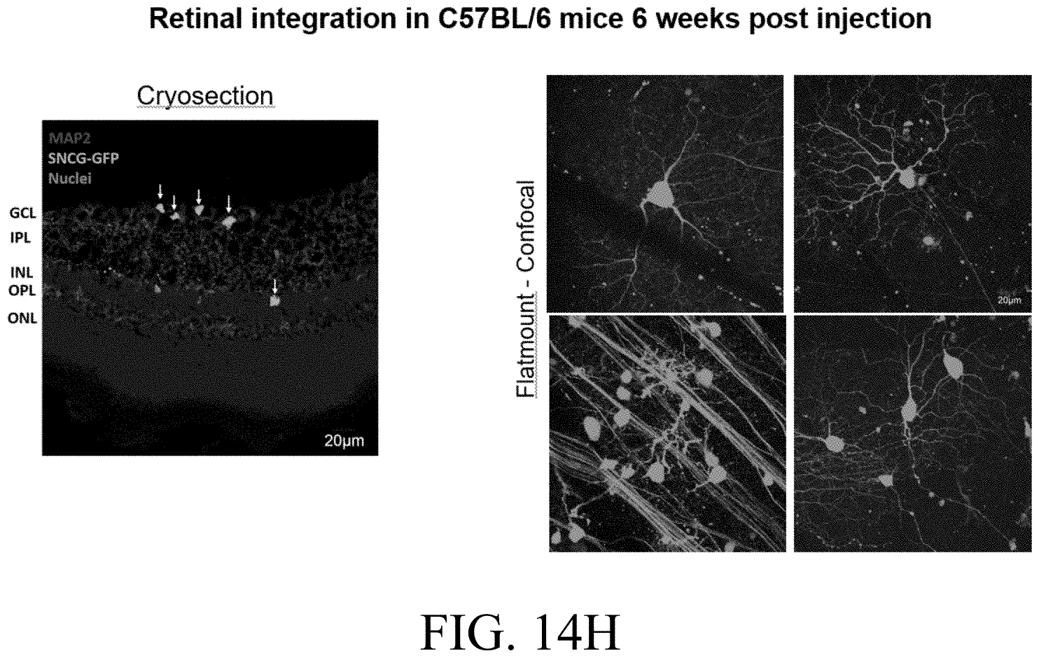

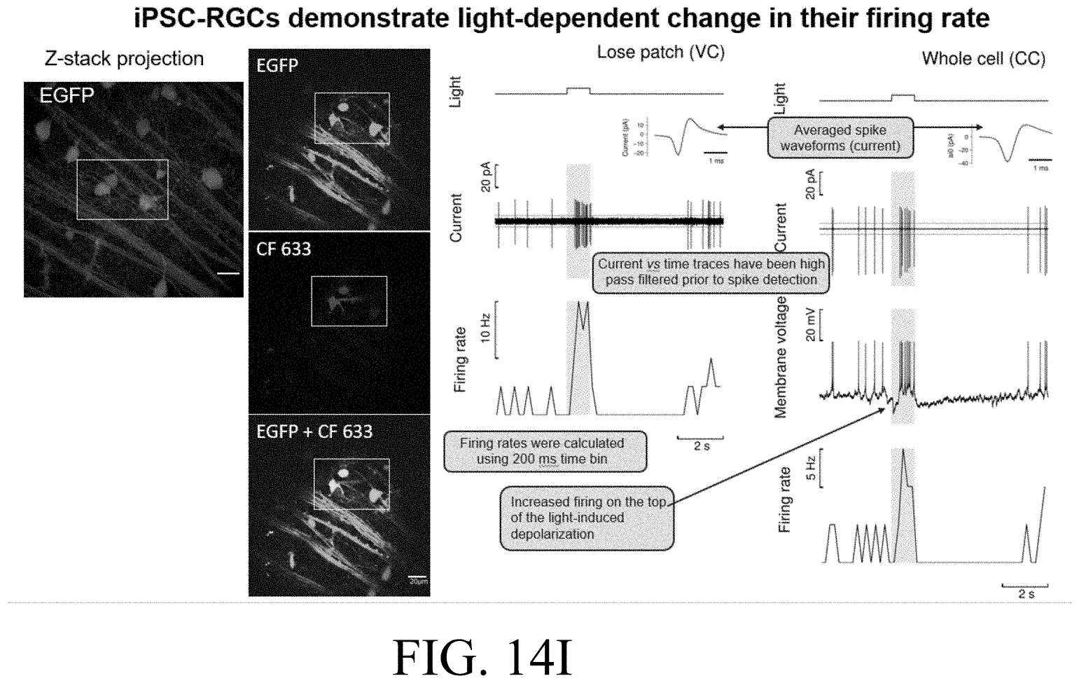

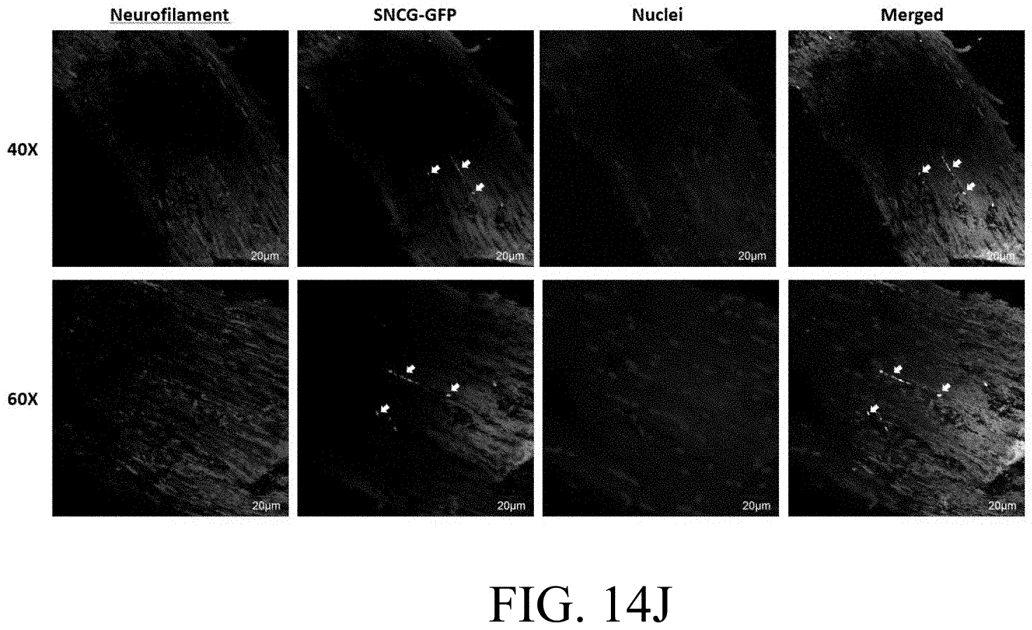

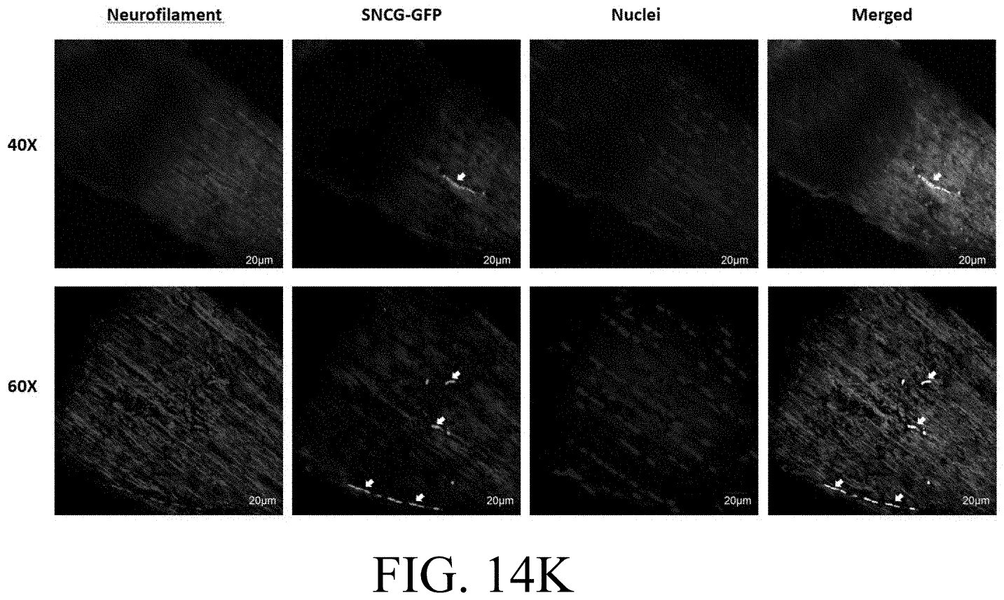

The information herein above has been applied clinically to mice as a model for therapeutic intervention as part of a cell replacement therapy. The schematic of iPSC-RGCs used for transplantation in a mouse retina is shown in FIG. 14A. Donor iPSCs were obtained from a donor and induced using the method discussed above to produce iPSC-RGCs. The cells were purified and, at day 36, the iPSC-RGCs were transduced with an AAV2.7m8-SNCG-GFP virus to fluorescently label the iPSC-RGCs. 2 μl of saline containing 1 million cells was then intravitrealously injected into the mouse retina of one eye in 10 mouse subjects. In each subject, the other eye was injected with saline as a control. Mice were then assessed at 2, 4, and 6 weeks following injection using Fundus imaging, SD-OCT and cSLO. (FIGS. 14B-14G) Integration was then determined using IHC on Flat mounts and sections of electrophysiology using whole cell recordings.

At six weeks, cryosections of the mice were taken to assess retinal integration after injection. FIG. 14H demonstrates that integration of the eGFP labeled iPSC-RGCs in the retinal ganglion cell layer was achieved. Flatmount retinal imaging (right image) shows transplanted iPSC-RGCs integrated into normal mouse retina, showing neuronal projections and dendrites. The transplanted iPSC-RGCs also showed light dependent change in firing. (FIG. 14I) The mice were continuously monitored for long term follow-up studies. At two months, Immunohistochemistry showed staining of transplanted iPSC-RGCs in mouse optic nerve sections two months post-transplantation. FIG. 14J-14K.

### Example 6

### Transplanted Human iPSC-Derived Retinal Ganglion Cells Integrate into Mouse Retinas and are Electrophysiologically Functional

Due to the asymptomatic progression of glaucoma, the majority of patients are unaware of the disease onset until it is severe, making it the leading cause of irreversible blindness worldwide. Consequently, there is an unmet need for the development of new strategies for the treatment of glaucoma. We investigated RGC replacement therapy as a treatment for ganglion cell loss. Human induced pluripotent stem cells (hiPSCs) were differentiated into mature, functional RGCs in vitro, labeled with AAV7m8-SNCG-eGFP, and transplanted intravitreally in wild type 4-month-old C57BL/6J mice. Survival of the transplanted hiPSC-RGCs was assessed by confocal microscopy and color fundus photography at 2-, 4-, and 6-weeks post-transplantation. Histological studies performed 2- and 5-months post-transplant confirmed the localization of the transplanted hiPSC-RGCs within the ganglion cell layer (GCL) of the retina. Two-photon live imaging of retinal explants and electrophysiological studies confirmed that the morphology and function of the transplanted hiPSC-RGCs were similar to native RGCs. The data provided herein disclose key strategies for enhancing the efficiency of stem cell replacement therapy and thereby advance potential treatments for neurodegenerative diseases including glaucoma and optic neuritis.

### Materials and Methods

Human iPSC Culture

Undifferentiated human induced pluripotent stein cells (hiPSCs) were derived and characterized as previously published showing a complete analysis of iPSC characteristics (68-70). The iPSCs were maintained in an iPSC medium: StemMACs iPSC-Brew XF (Miltenyi Biotec, catalog #: 130-107-086, North Rhine-Westphalia, Germany) containing 50× supplement (Miltenyi Biotec, catalog #: 130-107-087, North Rhine-Westphalia, Germany), and 5 ng/mL of basic fibroblast growth factor (bFGF; R&D Systems, catalog #: 233-FB, Minneapolis, Minn., USA) on 0.1% gelatin-coated dishes with irradiated mouse embryonic fibroblast (iMEFs). The components of StemMACs iPSC-Brew XF are provided in Table 6 below.

TABLE 6

| Media (500ml) | Stock conc | Final conc | Volume | |
|---|---|---|---|---|
| DMEM/F12 | | | 500 | ml |
| Sodium Bicarbonate 7.5% | | | 3.6 | ml |
| solution | | | | |
| L-Ascorbic Acid 2-Phosphate | 100x | 1% | 5 | ml |
| Sesquimagnes** | | | | |
| Insulin-Transferrin- Selenium | 100x | 2% | 10 | ml |
| bFGF solution | 4 ug/ml | 15 ng/ml | 1875 | ul |

### Retinal Progenitor Cell (RPC) Generation and Conditions

hiPSCs were cultured on 0.1% gelatinized plates containing iMEFs in 37° C., 5% O₂, and 5% CO₂ conditions. Cells were maintained until approximately 75% confluence, then feeder cells were depleted and approximately 1.5×10⁶ iPSCs were seeded in one well of a 6-well tissue culture dish (Corning, catalog #229106. Corning, N.Y., USA) coated with 1:100 diluted growth factor reduced Matrigel Growth Factor (GFR; Corning, catalog #: 354230, Corning, N.Y., USA). iPSCs were maintained in an iPSC: MEF-conditioned medium (80:20)+20 ng/mL of bFGF and 5 ng/mL of stable bFGF. The MEF-conditioned media was prepared by plating iMEFs onto 0.1% gelatin at a density of 20,000 cells/cm² in iPSC media. Two days post-plating, media was collected, filtered, and either used directly or cryopreserved for later use. iPSCs were maintained at 37° C. at 5% O₂ and 5% CO₂ until reaching 100% confluence then transferred to 37° C., 5% CO₂ overnight prior to induction.

On day 0. iPSC: MEF-conditioned media was changed to RPC induction media: DMEM/F12 (50:50: Corning, catalog #: 10-092-cm, Corning, N.Y., USA), 1% Penn/Strep (Life Technologies, catalog #: 10378-016, Carlsbad, Calif., USA), 1% Glutamine MAX (Life Technologies, catalog #: 35050-061, Carlsbad, Calif., USA), 1% NEAA (Life Technologies, catalog #: 11140-050, Carlsbad, Calif., USA), 0.1 mM 2-ME (Life Technologies, catalog #: 21985-O₂₃, Carlsbad, Calif., USA), 2% B27 supplement (w/o vitamin A; Life Technologies, catalog #: 12587-010, Carlsbad, Calif., USA), 1% N2 supplement (Life Technologies, catalog #: 17502-048, Carlsbad, Calif., USA), containing 2 μM XAV939 (X) (Wint inhibitor; R&D Systems, catalog #: 3748, Minneapolis, Minn., USA), 10 μM SB431542 (SB) (TGFβ inhibitor; R&D Systems, catalog #: 1614, Minneapolis, Minn., USA), 100 nM LDN193189 (L) (BMP inhibitor; R&D Systems, catalog #: 6053, Minneapolis, Minn., USA), 10 mM nicotinamide (Sigma-Aldrich, catalog #: N0636, St. Louis, Mo., USA), and 10 ng/mL IGF1 (R&D Systems, catalog #: 291-G1, Minneapolis, Minn., USA).

Cultures were fed daily for 4 days. On day 4, culture media was exchanged with RPC induction media containing: 2 μM XAV939, 10 μM SB431542, 100 nM LDN1931.89, 10 ng/mL IGF1, and 10 ng/mL bFGF (26).

### Retinal Ganglion Cell (RGC) Differentiation

Before RGC differentiation, the medium was changed daily using RGC induction media containing 250 ng/mL Shh (R&D systems, catalog #: 8908-SH, Minneapolis, Minn., USA). 100 ng/mL FGF8 (R&D Systems, catalog #: 423-F8, Minneapolis, Minn., USA) for 2 days.

For RGC differentiation on day 24, cells were manually crossed into small clusters with Leibovitz's medium containing 34 LM D-Glucose (Research Products International, catalog #: G32045-500, Mt. Prospect, Ill., USA) using the crosshatching technique as previously described (71), then re-plated at a density of 1.0×10⁵ cells/well of 6-well plate coated with 1:100 diluted growth factor reduced Matrigel Growth Factor in RGCs induction media containing: 100 ng/mL Follistatin 300 (R&D Systems, catalog #: 669-FO, Minneapolis, Minn., USA), 0.5 μM Cyclopamine (R&D Systems, catalog #: 1623/1, Minneapolis, Minn., USA), 3 μM DAPT (Stemgent, catalog #: 04-0041, Cambridge, Mass., USA), and 4.2 μM Y-27632 dihydrochloride (Rock inhibitor; R&D Systems, catalog #: 1254/1, Minneapolis, Minn., USA).

Media was changed 24 hours post-plating with RGC induction media containing: 100 ng/mL Follistatin 300 and 3 μM DAPT daily for 2 days. From day 27, media was changed to RGC induction media containing: 3 μM DAPT, 10 μM Rock inhibitor (Y27632), 5 μM Forskolin (Selleckchem, catalog #: S2449. Houston, Tex., USA), 400 μM N⁶,2′-O-Dibutyryladenosine 3′,5′-cyclic monophosphate sodium salt (cAMP; Sigma-Aldrich, catalog #: D0627, St. Louis, Mo., USA), 40 ng/mL BDNF (R&D Systems, catalog #: 248-BDB, Minneapolis, Minn., USA), 5 ng/mL NT4 (R&D systems, catalog #: 268-N4, Minneapolis, Minn., USA), and 10 ng/mL CNTF (R&D systems, catalog #: 257-NT, Minneapolis, Minn., USA). Media was changed every 2-3 days until day 36. After maturation (D36), the medium was exchanged every 3-4 days with RGC induction media containing: 3 μM DAPT and 10 μM Rock inhibitor (Y27632) (26).

### AAV Vector Design and Production

AAV expression cassettes were flanked by the AAV2 inverted terminal repeats. A pro-viral plasmid driving expression of enhanced green fluorescent protein (eGFP) (72) was driven by codon optimized SNCG (gamma-synuclein promoter) (73) and contains identical cis regulatory elements. AAV2.7m8-SNCG.eGFP vector was generated using previously described methods and purified with CsCl gradient by the CAROT research vector core at the University of Pennsylvania (74).

hiPSC-RGCs (Day 40) were transduced with AAV2.7m8 virus-containing SNCG-eGFP vector at a concentration of 1.92×10¹⁰ vg/ml. hiPSC-RGCs expressed green fluorescence as early as 48 hours post-transduction, the cells were washed with 1×PBS 3 days after the virus was applied and media was changed at least two times before being intravitreally injected (Day 50) into the eye of C57BL/6J mice.

### Animal Husbandry

Wild type C57BL/6J mice were obtained from the Jackson Laboratory (Bar Harbor, Me., USA) and raised in a 12-h light/dark cycle. Animals were housed at the University of Pennsylvania in compliance with the ARVO Statement for the Use of Animals in Ophthalmic and Vision Research as well as with institutional and federal regulations.

Flow Cytometry Analysis of hiPSC-RPCs and hiPSC-RGCs

hiPSC-RPCs and hiPSC-RGCs cultures were lifted using TrypLE Express (Invitrogen, catalog #: 12605-010, Waltham, Mass., USA) and collected by centrifugation at 1,600 rpm for 5 minutes at 4° C. The pelleted cells were resuspended in 1×PBS supplemented with 0.5% bovine serum albumin and 0.1% Na-Azide (FACS buffer). Cells were fixed in 4% paraformaldehyde (PFA) (v/v) for 15 minutes at room temperature (RT) followed by permeabilization using 0.5% Tween-20 (v/v) for 10 minutes at RT. Cells were incubated with various antibodies: anti-Ki67/MK167 (Novus Biologicals, catalog #: NB500-170SS, Littleton, Colo., USA), anti-Chx10 (Millipore, catalog #: AB9016, Burlington, Mass., USA), anti-CD90 (Thy1; Novus Biologicals, catalog #: AF2067, Littleton, Colo., USA), anti-sheep Alexa Fluor 405 (Abcam, catalog #: ab175676, Cambridge, UK), anti-BRN3 Alexa Fluor 594 (Santa Cruz, catalog #: sc-390780, Dallas, Tex., USA), anti-SNCG Alexa Fluor 488 (Santa Cruz, catalog #: sc-65979, Dallas, Tex., USA), and anti-RBPMS Alexa Fluor 647 (Novus Biologicals, catalog #: NBP273835AF647, Littleton, Colo., USA). Stained cells were analyzed using LSR B and LSRFortessa B at Penn Cytomics and Cell Sorting Resource Laboratory. Data were further analyzed using the FCS Express software.

Intravitreal Injections of hiPSC-RGC

4-month-old wild type C57BL/6J mice were anesthetized by isoflurane inhalation to effect (2-2.5%). For our injections, a 30-gauge needle was used to create a small incision at the limbus of the eye. Followed by 2 μL of 5×10⁵ live hiPSC-RGCs transduced with AAV2.7m8 virus-containing SNCG-eGFP vector was injected into the vitreous space of C57BL/6 mouse's right eye (n=16) using a 33-gauge 0.5″ blunt needle attached to a 5 μL Hamilton syringe (Hamilton Company, catalog #: 7803-05, Reno, Nev., USA). The contralateral eye injected with 2 μL of 1×PBS served as the experimental control. Following the injections, Prednisolone 1%/Gentamicin 0.3% ophthalmic ointment was applied topically on the eyes. The animals were placed on a heating pad until awake and the mice were monitored periodically for any signs of distress or complications.

### In-Vivo Ocular Fundus Imaging

Non-invasive retinal imaging was performed using Confocal Scanning Laser Ophthalmoscopy (“cSLO”, model Spectralis HRA, Heidelberg Engineering, Inc., Heidelberg, Germany), Color Fundus Imaging (“CFI”, model Micron III, Phoenix Instruments, Inc., Naperville, Ill.) and Spectral-Domain Optical Coherence Tomography (“SD-OCT”, model Envisu R2200 UHR, Bioptigen, Inc., Morrisville, N.C., USA) at 2-, 4-, and 6-weeks post-injection. Mice were anesthetized using Ketamine (100 mg/kg)/Xylazine (8 mg/kg), pupils dilated with a combination of 2.5% Phenylephrine/1% Tropicamide, followed by topical anesthesia to the cornea using 0.5% Proparacaine. Ocular protection against evaporative corneal desiccation during the imaging session was accomplished by using various combinations of artificial tears (Refresh, Genteal, and Balanced Salt Solution) and ocular eye shields. First, Wide-field (WF-55° FOV) and Ultra-wide field (UWF-105° FOV) cSLO images were collected in each eye with the plane of focus trained on the retina ganglion cell layer/nerve fiber layer region. BluePeak Autofluorescence (BAF-cSLO, 486 nm excitation/500-680 nm emission) images were collected with the optic nerve centrally positioned within the image field of view (FOV) frame.

This procedure was repeated with the Micron III system using the blue excitation and green auto-fluorescence emission channel. SD-OCT was performed last to assess the posterior pole retina and vitreous body structure. Posterior pole images included the posterior lens, vitreous body, retina, and choroid from a 50° FOV with the optic nerve central positioned. Following the imaging assessment, mouse eyes were covered with unmedicated ophthalmic ointment (Puralube Vet Ointment) and placed on a warmed heating pad (model 50-7220-F, Harvard Apparatus, Inc.) until recovered.

### Retinal Histology and RGC Quantification

A) Flattened whole mount: Eyes were harvested, fixed in 4% PFA at RT for 1 hour, and washed with 1×PBS, followed by isolation and dissection of the retina. Retinas were prepared as flattened whole mounts, washed with 1×PBS three times, and permeabilized in 0.5% Triton X-100 in PBS by freezing at −80° C. for 15 minutes. Retinas were incubated overnight at 4° C. in a humidified chamber with either BRN3 (Santa Cruz, catalog #: sc-6026, Dallas, Tex., USA), RBPMS (Sigma-Aldrich, catalog #: ABN1362, St. Louis, Mo., USA), Tubulin β 3 (TUJ1; BioLegend, catalog #: 801201, San Diego, Calif., USA), PSD95 (Cell Signaling Technology, catalog #: 3409S, Danvers, Mass., USA), VGLUT2 (Cell Signaling Technology, catalog #: 14487S, Danvers, Mass., USA), Ku80 (Cell Signaling Technology, catalog #: 2180S, Danvers, Mass., USA), or Human Nuclear Antigen (Novus Biologicals, catalog #: NBP2-34342, Littleton, Colo., USA).

The next day, the primary antibody was removed, and the retinas were washed with 1×PBS five times for 5 minutes each, followed by incubation with appropriate secondary antibodies: anti-goat Alexa Fluor-594 (ThermoFisher Scientific, catalog #: A11080, Waltham, Mass., USA), anti-mouse Alexa Fluor-594 (ThermoFisher Scientific, catalog #: A11020, Waltham, Mass., USA), and anti-rabbit Alexa Fluor-594 (ThermoFisher Scientific, catalog #: A21442, Waltham, Mass., USA) for 1 hour at RT. The secondary antibody was discarded, and the retinas were washed with 1×PBS seven times for 5 minutes each. The coverslips were then mounted on slides using proLong Gold antifade reagent with DAPI (4′,6-diamidino-2-phenylindole) (Invitrogen, catalog #: P36935, Waltham, Mass., USA). Slides were observed under an Invitrogen EVOS M5000 fluorescent microscope and Olympus FV1000 Confocal laser scanning microscope and images were captured using 4×, 10×, and 40× objectives with the use of appropriate filters and lasers. Images were analyzed using ImageJ software.

B) Cryosections: Enucleated eyes were fixed in 4% PFA overnight at 4° C. For cryo-sectioning, eyecups were dissected, and the lens was extracted. The retinal cups were first transferred to 15% sucrose solution for 1 hour at 4° C. and were then again transferred to 30% sucrose solution and incubated overnight at 4° C. Eyes were embedded in optimal cutting temperature (OCT) compound and were frozen in place on dry ice blocks and stored at −20° C. for long-term storage. Using a cryostat, 14 μm sections of the retinal cups were generated. For immunohistochemistry, the cryo-sections were washed with 1×PBS three times for 5 minutes each. The slides were incubated with blocking buffer (1% BSA, 0.1% Triton X-100, and 1% normal donkey serum in 1×PBS) in a humidified chamber for 1 hour at RT. The slides were then incubated overnight at 4° C. using the following antibodies: BRN3 (Santa Cruz, catalog #: sc-6026, Dallas, Tex., USA), MAP2 (Santa Cruz, catalog #: sc-74421, Dallas, Tex., USA), and Synapsin I (Sigma-Aldrich, catalog #: 574777, St. Louis, Mo., USA).

The next day, samples were washed with 1×PBS three times for 5 minutes each, followed by incubation with appropriate secondary antibodies: anti-goat Alexa Fluor 594 (ThermoFisher Scientific, catalog #: A11080, Waltham, Mass., USA), anti-mouse Alexa Fluor 594 (ThermoFisher Scientific, catalog #: A 11020, Waltham, Mass., USA), and anti-rabbit Alexa Fluor-594 (ThermoFisher Scientific, catalog #: A21442, Waltham, Mass., USA) for 1 hour at RT. The coverslips were then mounted on slides using proLong Gold antifade reagent with DAPI (4′,6-diamidino-2-phenylindole) (Invitrogen, catalog #: P36935, Waltham, Mass., USA). Slides were observed under an Olympus FV1000 Confocal laser scanning microscope and images were captured using 40× objective with the use of appropriate filters and lasers. Images were analyzed using ImageJ software.

Voltage Reading of Transplanted hiPSC-RGCs

All animal procedures conformed to National Institutes of Health guidelines for animals in research and of the University of Pennsylvania Committee for the Care and Use of Animals. Mice were sacrificed by the injection of a lethal dose of ketamine/xylazine mixture (100 μg/g ketamine and 10 μg/g xylazine) followed by cervical dislocation, and eyes were enucleated and dissected in a Petri dish filled with Ames solution (Sigma-Aldrich Inc., Burlington, Mass., USA) maintained at room temperature and continuously oxygenated with a mixture of 95% O₂ and 5% CO₂. Retinas were flat-mounted ganglion cells side up in the recording chamber perfused with oxygenated Ames solution maintained at 35-37° C. using TC-344C two-channel temperature controller (Warner Instruments, Holliston, Mass., USA). All procedures were done under dim red-light illumination.

Two-photon imaging using Olympus FV1000 MPE Multiphoton Laser Scanning Microscope (Olympus Corporation of the Americas, Center Valley, Pa., USA) was employed to identify, image, and target eGFP-expressing hiPSCs in a mouse retina. The wavelength for two-photon excitation was set at 920 nm. All pipette manipulations were done using a different IR viewing system providing regular video frame rates. It included a Dage-MTI CCD 72 IR camera and controller (Dage-MTI Inc., Michigan City, Ind., USA), a Sony Trinitron TV monitor (Sony Corporation of America, New York, N.Y., USA), and a #87 IR filter (Lee Filters, Andover, England) installed in the microscope's light path for a regularly transmitted light illumination. If needed imaging could be briefly switched back to two-photon mode right before touching the cell membrane with the pipette tip to confirm that the red die CF 633 filled pipette is indeed positioned next to the targeted EGFP expressing hiPSC. At the end of the electrophysiological recording two-photon imaging was used once again to confirm that the targeted cell was filled with the CF 633 die. Images were processed using Olympus Fluoview software and ImageJ (Rasband, W. S., ImageJ, U. S. National Institutes of Health, Bethesda, Md., USA, available on the world wide web at imagej.nih.gov/ij/, 1997-2018).

The hiPSCs were recorded in the cell-attached and whole-cell modes. First, an Ames-filled pipette was used to break through the inner limiting membrane and clean a small area around a cell membrane region to be patched. After that, it was replaced with another Ames-filled pipette used to record spiking activity from the targeted cell in the cell-attached (also known as the loose patch) configuration in the voltage-clamp mode. In this configuration, a seal is formed between the pipette and cell membrane although it is not required to be as tight as a seal used in patch-clamp recording, and a cell membrane is not ruptured. Finally, a pipette filled with intracellular solution (112 mM K-Gluconate, 12 mM NaCl, 2 mM MgCl₂, 1 mM EGTA, 10 mM HEPES, Ph adjusted to 7.4 using 26 mM of KOH) was used to achieve a tight seal (typically above 2-3 GΩ) with the cellular membrane, a whole-cell configuration established by rupturing a piece of the membrane inside the pipette with a brief pulse of suction (in the voltage-clamp mode), and cellar responses to depolarizing inputs caused by light stimulation or current injection were recorded in the current-clamp mode. All pipettes were fabricated using a Sutter P-87 puller (Sutter Instrument Company, Novato, Calif., USA). Resistance of the Ames-filled pipettes was around 5-7 MΩ, and the resistance of patch-pipettes was around 7-9 MΩ.

Light stimulation included calibrated full-field flashes of 500 nm light delivered from below the stage using a custom LED-based system. Cellular responses were amplified using Warner Instruments PC-505B patch-clamp amplifier, Axon Digidata 1400 digitizer, and Clampex software (Molecular Devices, San Jose, Calif., USA) was used to record data on the computer hard disk and to control light and current stimulation. Custom Matlab-based code was used for data analysis (Math Works, Natick, Mass., USA). Because of fast action potential spike kinetics (spike duration typically is less than 3 ms) and a real-world limitation of the current-clamp system, spikes can be reliably detected from current vs time traces recorded both in voltage and current clamp modes. To detect spikes these traces were high pass filtered (4 pole Butterworth filter, cutoff frequency 250 Hz), and a 4 STD detection threshold was used. Reported voltage traces recorded in the current-clamp mode were not corrected for the liquid junction potential (UP), estimated to be around 15 mV (to correct for UP 15 mV should be subtracted from the reported values).

### Avon Analysis

Optic nerves were isolated, processed, and embedded in paraffin. 5 μm longitudinal paraffin sections of the optic nerve were deparaffinized and rehydrated, followed by incubation with blocking buffer (1% BSA, 0.1% Triton X-100, and 1% normal donkey serum in 1×PBS) in a humidified chamber for 1 hour at RT. The slides were then incubated overnight at 4° C. with Neurofilament antibody (Sigma-Aldrich, catalog #: MAB5266, St. Louis, Mo., USA). The next day, samples were washed with 1×PBS three times for 5 minutes each, followed by incubation with anti-mouse Alexa Fluor-594 (ThermoFisher Scientific, catalog #: A11020, Waltham, Mass., USA), staining was performed for 1 hour at RT. The coverslips were then mounted on slides using proLong Gold antifade reagent with DAPI (4′,6-diamidino-2-phenylindole) (Invitrogen, catalog #: P36935, Waltham, Mass., USA). Slides were observed under an Olympus FV1000 Confocal laser scanning microscope and images were captured using 60× objective with the use of appropriate filters and lasers.

### Statistical Analysis

The total number of eGFP+hiPSC-RGCs detected in mouse retinas were quantified using Ilastik cell density counting software (Heidelberg, Germany) (75) and validated by manual counting in ImageJ software (NIH, Bethesda, Md., USA).

### Results:

In Vitro Human iPSC Differentiation Generates Pure Populations of RPCs and RGCs

Clinical translation of hiPSC-RGC transplantation in an in vivo model is highly dependent on the availability of a scalable differentiation protocol to efficiently generate mature and functional RGCs in an expedited and less labor-intensive manner. Described herein is a two-step/stage differentiation technique, in which hiPSCs are initially differentiated to retinal progenitor cells (RPCs) via the step-wise addition of small molecules and peptide modulators in a chemically defined media. This is followed by step 2, a cross-hatching technique on day 24 that allows for the expansion and differentiation of RPCs into functionally mature iPSC-RGCs. The detailed and stepwise description of the hiPSC-RGC differentiation protocol is outlined above.

The hiPSC lines obtained from de-identified patient-derived normal Caucasian and African American donors were differentiated into hiPSC-RPCs and hiPSC-RGCs (28)). Prior to transplantation, the purity of hiPSC-RPC and hiPSC-RGC populations were assessed using fluorescence-activated cell sorting (FACS) on day 15 and day 35, respectively. Ki67 and Chx10 were used for the characterization of hiPSC-RPCs. BRN3/POU4F, gamma Synuclein (SNCG), CD90/Thy1, and RNA-binding protein with multiple splicing (RBPMS) were used for the identification of hiPSC-RGCs.

hiPSC-RPCs generated using our method stain positive for Ki67 (95.5%) and Chx10 (82%) markers (29). Similarly, hiPSC-RGCs stain positive for BRN3 (87%), SNCG (93%), CD90 (85.5%), and RBPMS (22.5%) (29). Our differentiation protocol consistently generates RPCs and RGCs that are highly positive for the respective differentiation markers identified in Table 7A-7B. The hiPSC-RGCs can be maintained in culture for over 3 months with minimal cell loss, and therefore, serve as a readily available source of RGCs for transplantation studies.

TABLE 7A
Flow-cytometric analysis of patient-derived iPSC for RPCs

| PennID for iPSC | | | RPC Markers | |
|---|---|---|---|---|
| Line | Race | Gender | Ki67 | Chx10 |
| 1) Penn123i-SV20 | European | Male | 90% | * |
| | American | | | |
| 2) Penn087i-38-1 | European | Male | 100% | 73.5% |
| | American | | | |
| 3) Penn019i-136-6 | European | Male | 77.5% | 80% |
| | American | | | |
| 4) Penn024i-370-3 | African | Female | 95.5% | 82% |
| | American | | | |

hiPSC-RPCs were analyzed by FACS on day 15 for ki67 and Chx10 markers
*Indicative of negligible/low presence of markers

TABLE 7B

| | | | RGC Markers | | | |
|---|---|---|---|---|---|---|
| PennID for iPSC Line | Race | Gender | Brn3 | RBPMS | SNCG | CD90 |
| 1) Penn123i-SV20 | European American | Male | 71% | 46% | * | * |
| 2) Penn087i-38-1 | European American | Male | 49.5% | 4% | 25% | 87% |
| 3) Penn019i-136-6 | European American | Male | 87% | 22.5% | 93% | 85% |
| 4) Penn039i-63-1 | African American | Female | 85% | 2.3% | 78% | 58% |
| 5) Penn059i-555-1 | African American | Female | 83% | 34% | 84% | 91% |

hiPSC-RGCs were analyzed by FACS on day 35 for RGC-specific markers such as Brn3, RBPMS, SNCG, and CD90
*Indicative of negligible/low presence of markers

Transplanted hiPSC-RGCs were Detected in the Murine Retina

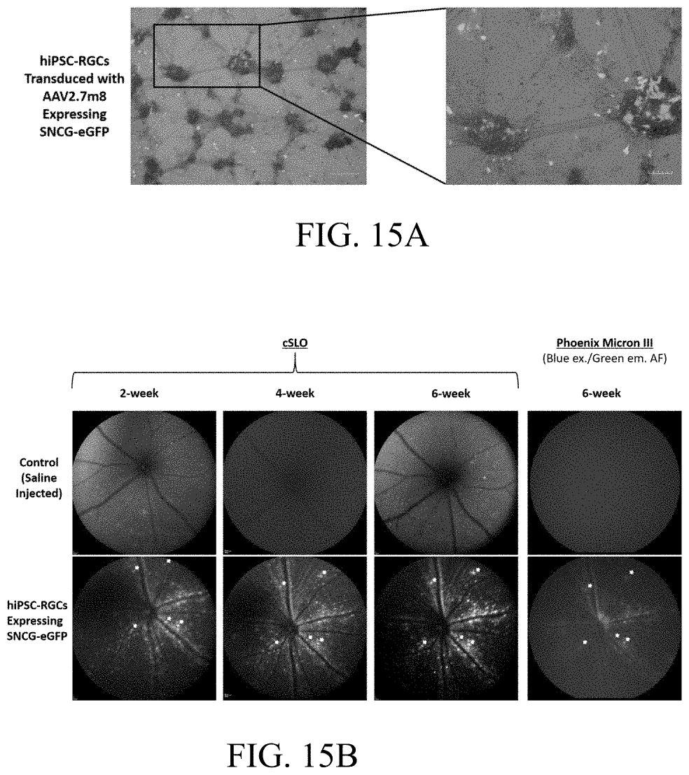

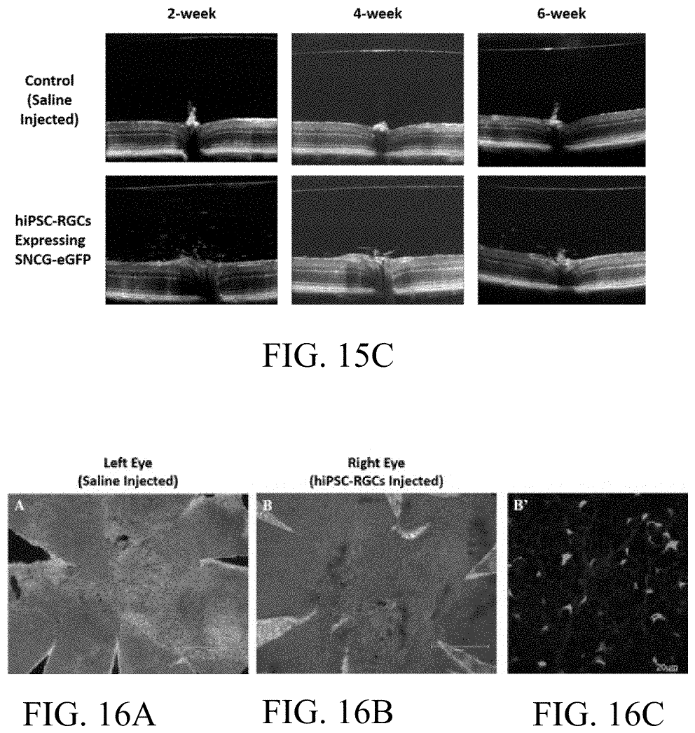

For transplantation into the murine retina, we fluorescently labeled hiPSC-RGCs by transduction with AAV2.7m8-SNCG-eGFP, which expresses eGFP under the control of an RGC-specific gamma Synuclein (SNCG) promoter (30). Labeled hiPSC-RGCs expressed green fluorescence as early as 48 hours post-transduction (FIG. 15A).

Owing to the high purity of transduced hiPSC-RGCs, 5×10⁵ viable cells (˜Day 50) were intravitreally injected into the eye of 4-month-old wild type C57BL/6J mice (n=16. The contralateral eye was injected with 1×PBS as an experimental control. The mice were subsequently monitored for any signs of distress or complications. In vivo fundus images were taken using wide-field (WF) and ultra-widefield (UWF) blue autofluorescence-confocal scanning laser ophthalmology (BAF-cSLO) and spectral-domain optical coherence tomography (SD-OCT) to assess the presence of eGFP+ cells at 2-, 4-, and 6-weeks post-injection (FIG. 15B-15C).

Following intravitreal delivery, eGFP+hiPSC-RGCs were detected within the ganglion cell layer of the murine retina as early as 2-weeks post-injection seen as punctate hyperfluorescent foci in the BAF-cSLO and color fundus imaging (CFI) (FIG. 15B). We did not observe any gross abnormalities in the fundus images of any saline- or hiPSC-RGCs injected eyes. SNCG-eGFP+ donor cells were uniformly distributed across the pan-field of view of the mouse retina, however, they were predominantly localized adjacent to retinal veins than retinal arteries. The nearest neighbor index (NNI) (the ratio of mean nearest observed distance between eGFP+ cells (Nnd=˜3 μm) to mean random expected distance) was used to quantify the donor cell spatial distribution pattern. If the index is greater than 1, the cells are determined to be well dispersed, whereas if the index is less than 1, the cells are considered to be clustered. In our study, NNI was determined to be 0.511 (n=3), hence the transplanted hiPSC-RGCs were partially clustered within the mouse retina (31-33).

On average there were 672 (range 230 to 1535) hiPSC-RGCs detected following intravitreal injections (Table 8). The average transplantation efficiency was 0.134%. Furthermore, based on recent studies, transplantation is considered successful if 0.1% of the total transplanted donor RGCs survive in vivo (21, 24). In that case, the transplantation experiments were successful in n=9/16 mice.

TABLE 8
Number of eGFP+ donor hiPSC-RGCs detected per mouse retina

| | | | Number of eGFP+ hiPSC- |
|---|---|---|---|
| | PennID for | | RGCs Detected per Mouse |
| Mouse ID | iPSC Line | Gender | Retina |
| 1) | Penn123i-SV20 | Male | 1098* |
| 2) | Penn123i-SV20 | Male | 1535* |
| 3) | Penn123i-SV20 | Male | 816* |
| 4) | Penn087i-38-1 | Female | 702* |
| 5) | Penn087i-38-1 | Female | 403 |
| 6) | Penn087i-38-1 | Female | 787* |
| 7) | Penn087i-38-1 | Female | 375 |
| 8) | Penn087i-38-1 | Female | 753* |
| 9) | Penn087i-38-1 | Male | 428 |
| 10) | Penn087i-38-1 | Male | 1166* |
| 11) | Penn087i-38-1 | Male | 282 |
| 12) | Penn087i-38-1 | Male | 445 |
| 13) | Penn087i-38-1 | Male | 230 |
| 14) | Penn059i-555-1 | Female | 537* |
| 15) | Penn059i-555-1 | Female | 525* |

| | |
|---|---|
| Average | 672 |

*Indicative of hiPSC-RGCs detected in a mouse retina that were more than 0.1% of the total donor cells injected and hence considered as successful transplantation.

SD-OCT images confirm the structural integrity of the mouse retina following intravitreal injections (FIG. 15C). We evaluated the vitreous cavity for the presence of abnormal scattering that evidences donor hiPSC-RGC remnants and/or the presence of immune cells at 2-, 4-, and 6-weeks following intravitreal injections.

Detection of the Transplanted hiPSC-RGCs within the Murine Retina

The eyes of wild type mice injected with hiPSC-RGCs were enucleated 2- and 5-months post-transplantation. Immunohistochemistry (IHC) analysis of retinal sections (n=3) was performed to determine the localization of the transplanted hiPSC-RGCs within the host retina. Retinal cryosections were co-stained with RGC-specific markers including BRN3, MAP2, and Synapsin I to confirm the identity of the transplanted cells.

### BRN3, MAP2, and Synapsin I

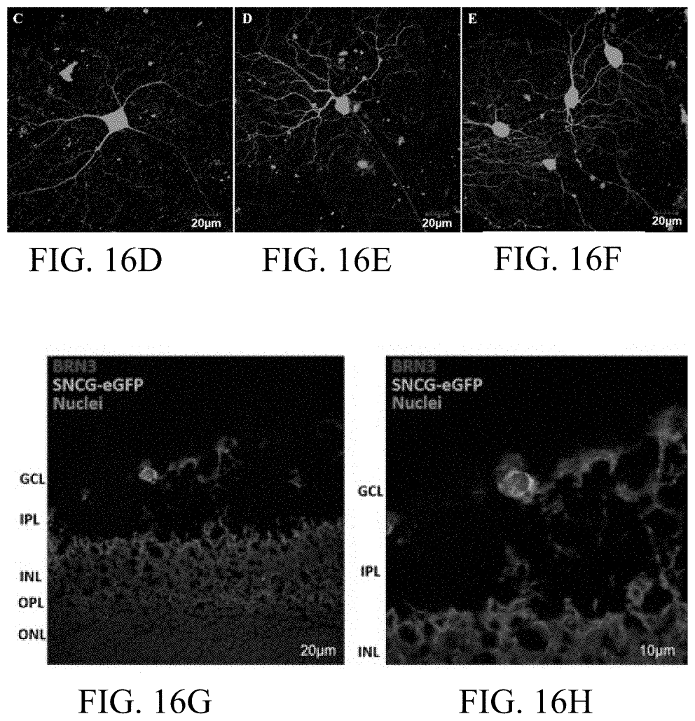

The majority of the transplanted hiPSC-RGCs were found within the ganglion cell layer (GCL) of the mouse retina interweaved with host ganglion cells (FIG. 16B-16C), while a few displaced RGCs (dRGCs) migrated into the inner nuclear layer (34) (FIG. 16G-16J). Transplanted hiPSC-RGCs exhibited morphological characteristics similar to endogenous RGCs, with long complex dendritic stratification and some with short-range neurite outgrowth (FIG. 16D-16F). Furthermore, the donor cells had dendritic extensions into the inner plexiform layer as seen in the side projection of two-photon Z-stack images acquired from the live murine retinal samples, thus indicating that the hiPSC-RGCs are forming projections to the appropriate cell layer within the host retinal circuit.

Characterization of the Transplanted hiPSC-RGCs within the Murine Retina

Whole-mount immunohistochemistry (n=8) was performed on the retinas isolated from enucleated eyes 2- and 5-months after injection with hiPSC-RGCs. Flattened retinas were counter-stained for RGC-specific markers such as BRN3, RBPMS, MAP2, and TUJ1, including synaptic markers like VGLUT2 and PSD95.

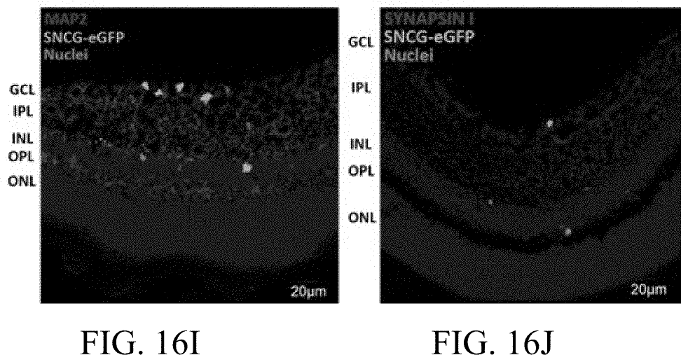

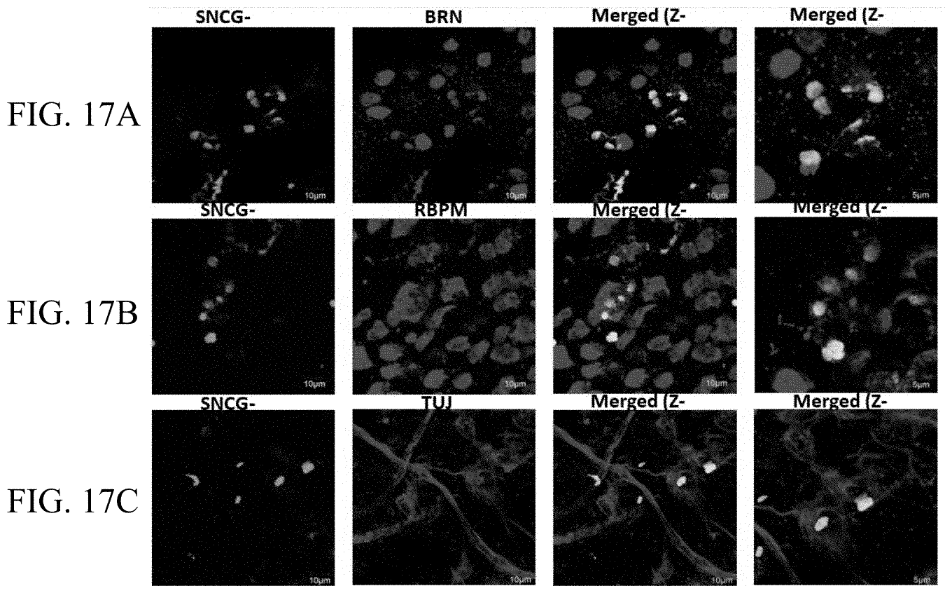

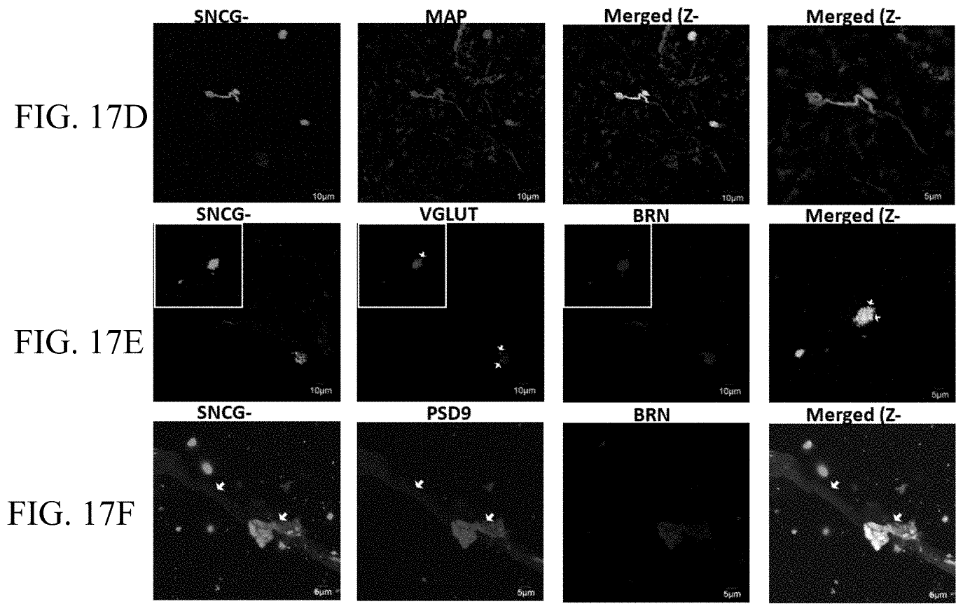

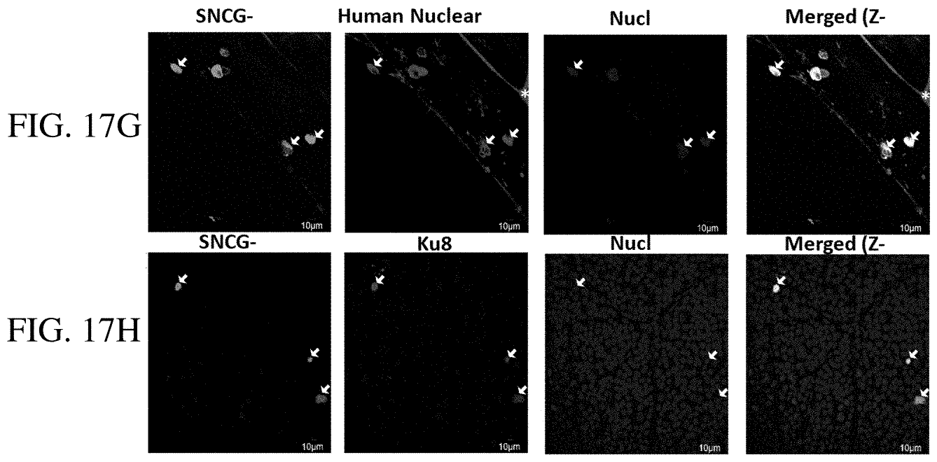

All the SNCG-eGFP+hiPSC-RGCs detected in the mouse retinas expressed BRN3 (FIG. 17A) and RBPMS (FIG. 17B). MAP2 and TUJ1. Synaptic markers (VGLUT2 and PSD95).

Human nuclear antigen was used to label the donor hiPSC-RGCs to confirm the donor origin of the transplanted cells; all SNCG-eGFP+ cells were also human nuclear antigen and Ku80-positive, suggesting that there was no material exchange between transplanted hiPSC-RGCs and the host RGCs (FIG. 17G-17H).

Function of the Transplanted hiPSC-RGCs

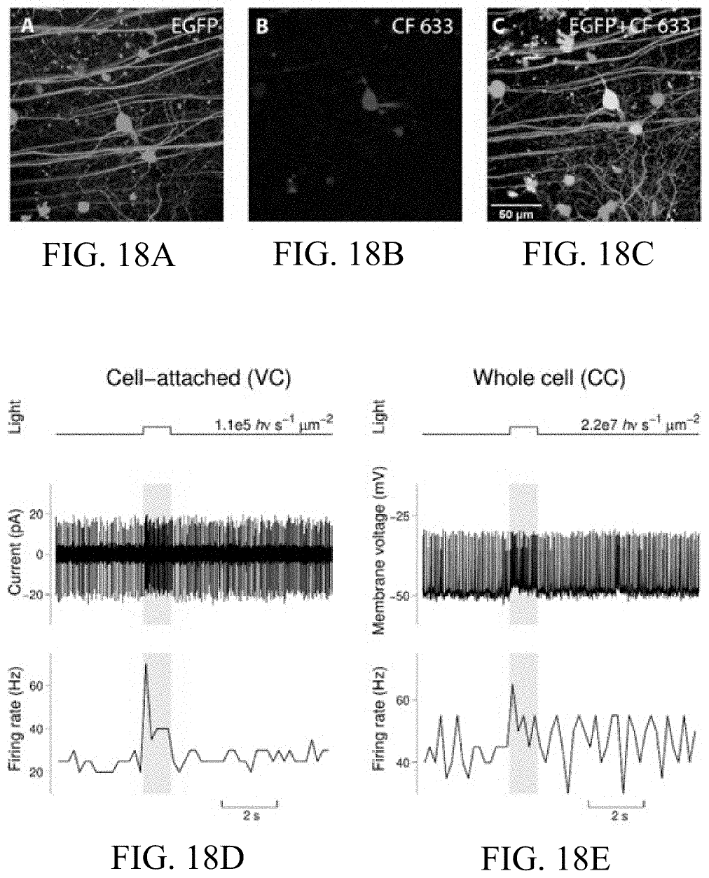

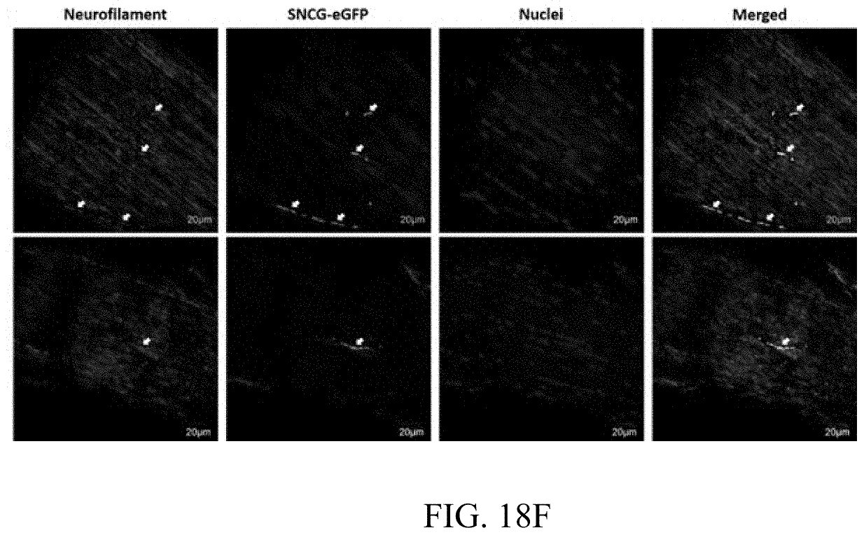

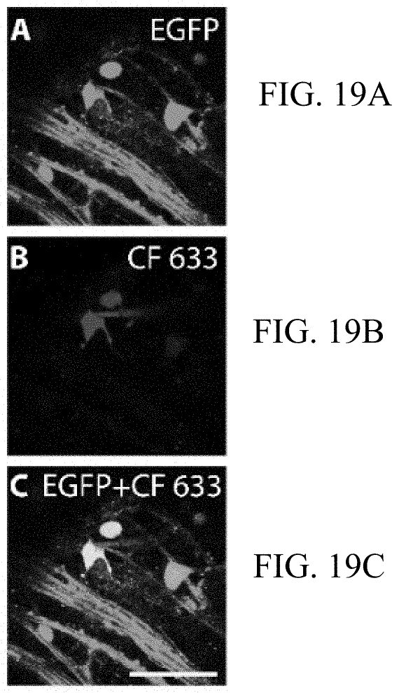

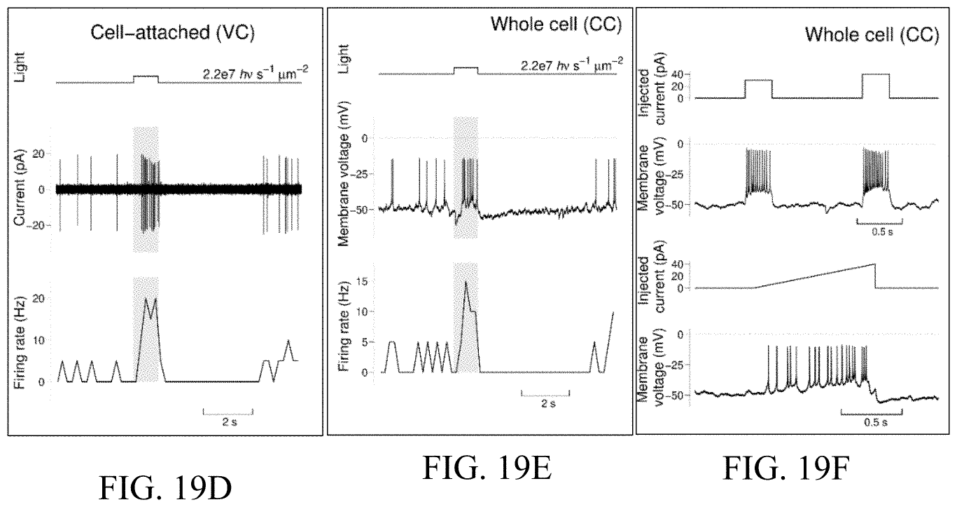

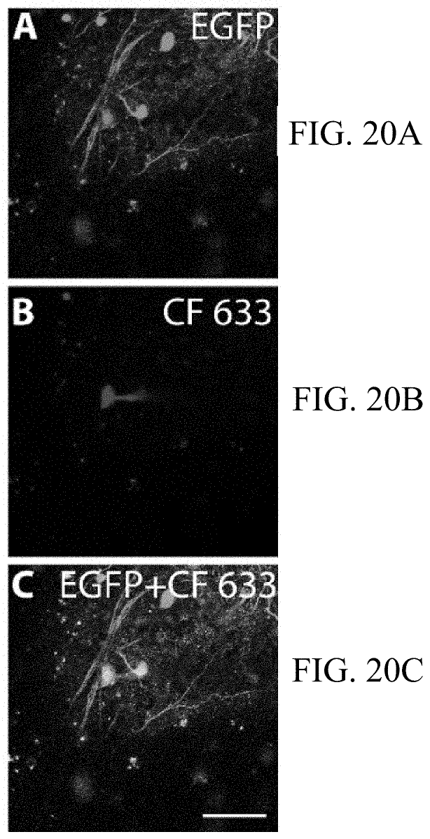

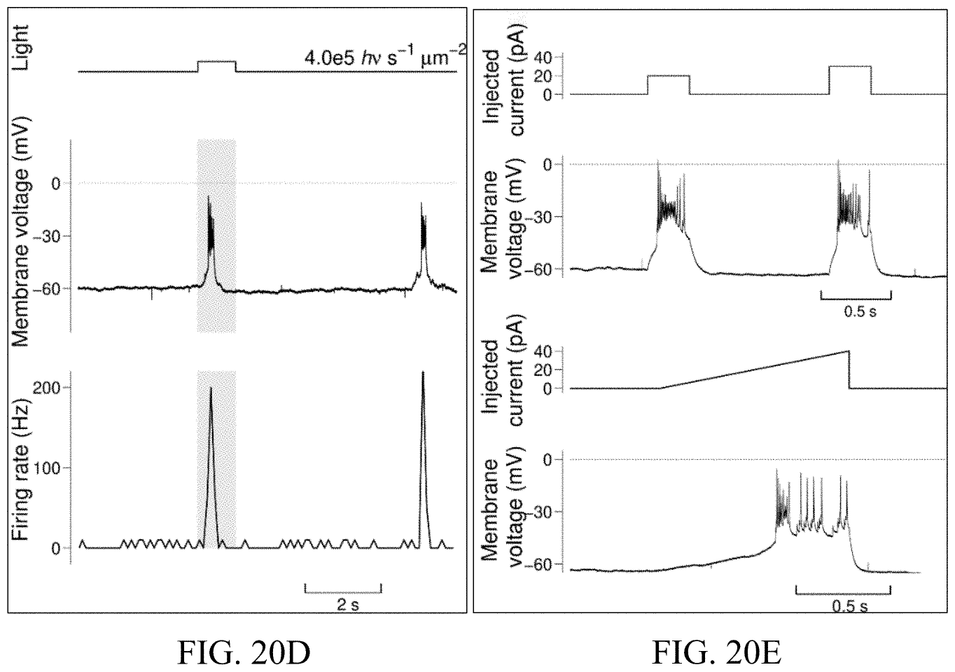

Transplanted retinas from mice aged 9-months (n=4) were used for live two-photon imaging and electrophysiological studies. Two-photon live images revealed an elaborate dendritic arborization of transplanted hiPSC-RGCs and synaptic connections resembling host RGCs suggesting successful integration in the host retina (FIG. 18A-18C). Functional integration was assessed by electrophysiologic responses to full-field photopic stimuli. Light responses were first recorded in the cell-attached (loose patch) configuration in the voltage-clamp mode (FIG. 18D) before rupturing the membrane and establishing the whole-cell configuration, and in the current-clamp mode in the whole-cell configuration (FIG. 18E). Of the six recorded hiPSC-RGCs, two cells produced light responses with elevated baseline firing (FIG. 18), two cells generated light responses with very low baseline firing (FIGS. 19D and 19E), and two cells produced increased firing over large, often spontaneous depolarizations. For one of the last two cells, the probability of synchronization between flash and spike firing was around 60%, well above 10% which would be expected if spike firing was completely random. Depolarizations and spike firing observed for the other cell appear to be mostly spontaneous (FIGS. 19F and 20E).

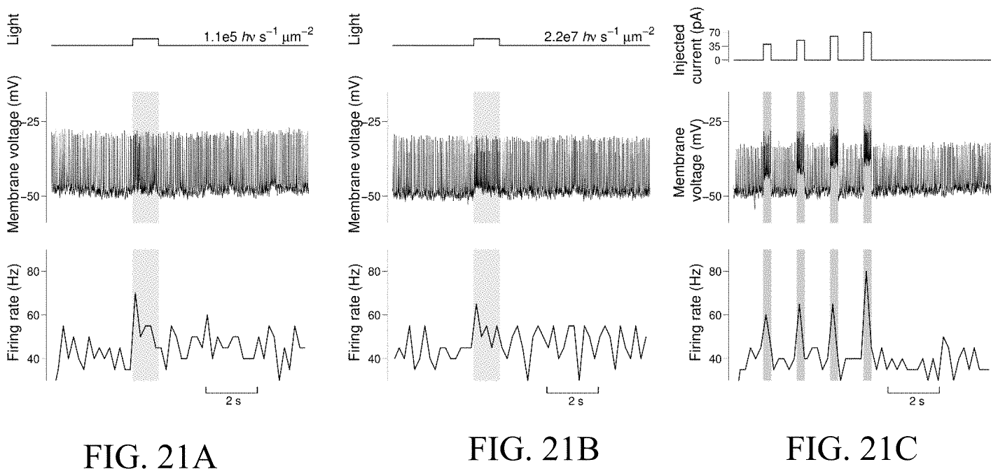

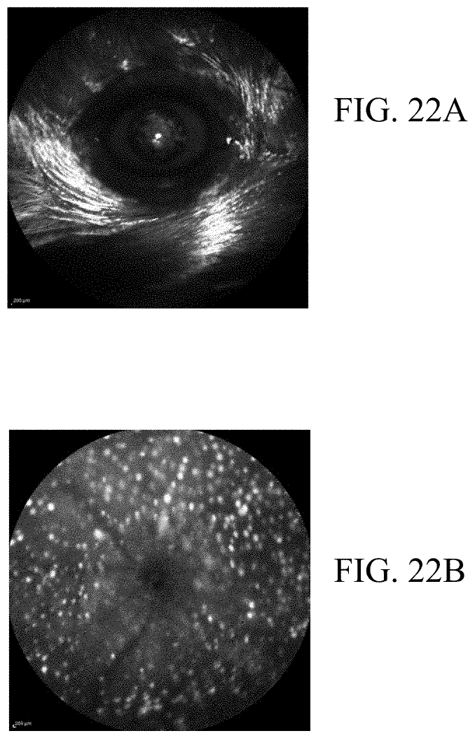

The hiPSC-RGCs firing rate increase in response to depolarizing stimuli appears to diminish upon repeated stimuli. The data indicates that such a decrease in responsivity was not caused by the dilution of the intracellular content by the pipette solution because it was detected in the cell-attached as well as in the whole-cell configurations. It was also observed that a brighter flash delivered later on could cause a larger membrane depolarization but a smaller increase in the firing rate compared to the preceding dim flash (FIGS. 21A and 21B). Moreover, a small depolarization in response to the first light flash could produce a larger increase in the firing rate than much larger depolarizations caused by current injections, when current injections were done after repeated light stimuli (FIG. 20A-20C). FIGS. 18 and 20 present data from the same cell and a comparison of Z-stack projections before and after pipette recording. FIG. 18C does not show any broken/missing cellular processes, indicating that the observed decrease in responsivity is not due to any physical damage caused to the cell by pipette manipulations. Progressively smaller increases in the firing rate in response to depolarizations caused by light exposures or current injections were observed for all recorded cells. Additionally, the responsivity at least partially recovered when cells were rested without stimulation for 30 s or longer.

To improve vision, axons of functionally integrated donor hiPSC-RGCs need to extend all the way to synapse with targets in the visual cortex of the brain. Herein, we processed optic nerves of mouse eyes intravitreally injected with hiPSC-RGCs (n=16), and detected graft axonal projections of eGFP+hiPSC-RGCs in the optic nerve head (ONH). The cells are also shown herein to express neurofilament marker (FIG. 18F). Neurofilament proteins are essential for the maintenance of the structure and shape of the neurons, and they are involved in the regulation of signal transduction and synaptic plasticity of the axons (35). Furthermore, this indicates that the donor hiPSC-RGCs were aligned with and followed along with host RGC axons.

### DISCUSSION

Transplantation experiments of immature retinal sheets into the eye can be traced as far back as 1964 (36). However, the success of ganglion cell transplantation is highly dependent on the purity of the donor retinal cells. The efficiency of current retinal differentiation protocols across different cell lines such as mESC, hESC, miPSC, and hiPSC is highly variable and often results in low yield and/or low purity of retinal cell populations (37-39). Early attempts included co-culturing mESCs with extracted mouse retinal tissue to promote the differentiation towards retinal cell fate, but this resulted in few cells that expressed RGC-like markers (40-44). RGCs derived from cultured embryoid bodies that formed optic vesicle-appearing neurospheres were detected from days 20-50 and were reported to express BRN3 and Calretinin. This technique was reported in both hiPSCs and ESCs (45, 46). This method was then expanded to develop optic cup-like structures from mESCs and hESCs and has been reported to recapitulate in vivo retinal development (47, 48). RGCs have also been differentiated from miPSC- and mESC-derived 3D-retinal tissues but resulted in low cell purity (8% Brn3a+ and 5% RBPMS+) and thus required further enrichment with Thy1.2 based magnetic associated cell sorting (24).

In recent years, the use of iPSCs for cell therapies has garnered a lot of attention, as iPSCs are an excellent source of stem cells that can be directed to generate every key cell type in the eye and can be used to treat neurodegenerative diseases such as glaucoma (49). Furthermore, using human iPSCs for the treatment of diseases can overcome some of the ethical concerns associated with the use of human embryonic stem cells (50). Patient-derived iPSCs are an ideal source of donor cells as they circumvent host vs. donor transplant immunological rejection. The differentiation protocol presented herein consistently produces large populations of hiPSC-derived neuronal cells that express over 70% of RGC-specific markers such as BRN3, Thy1, and SNCG to date.

However, it should be noted that although these markers are specific to RGCs within the retina, they are expressed by a variety of different non-retinal neuronal cells such as CNS neurons and motor neurons. Therefore, if necessary, different techniques to confirm in vitro cell identity by RNA sequencing analysis, DNA methylation patterns of RGC-specific genes, microarrays, or transcriptomic and proteomic approaches can be employed. For translational studies, the purity of donor RGCs can be determined to reduce the risk of teratoma formation from undifferentiated or heterogeneous cell populations. To achieve successful transplantation, different RGC subtypes can be identified, as each of the 20 different RGC subtype identified to date has a unique pre- and post-synaptic function.

We performed in vivo studies in a wild type adult C57BL/6J mouse model. The use of murine models for transplantation studies considerably reduces the risk, time, and costs of advancing the therapy into human clinical trials. hiPSC-RGC transplantation into the murine eye was administered via intravitreal injection as it is the most targeted route of cell delivery to the retina without disrupting the blood-retinal barrier and it bypasses systemic exposure (51). However, since the vitreous space is a large cavity, it is difficult to control the dispersion of the injected cells to a specific area of the retina and in some cases, the donor cells adhere to the posterior lens capsule and ciliary body (52).

In our current transplantation methodology, we recovered about 50% of viable cells in the single cell dissociation step. Optimization of this step can be done to lessen cell loss and efficiently break up the cell clusters into single cells to perform injections with a higher number of live and healthy RGCs. Furthermore, cell sorting by FACS to remove dead cells after cell dissociation will improve hiPSC-RGC survival post-transplantation and their integration in the mouse retina. However, numerous cues influence the survival rate of the donor cells such as the innate and adaptive immune system of the recipient animal, and factors in the ocular environment within the mouse such as the blood flow, oxygen, and nutrient supply which all have a direct and indirect role in the eventual homing and integration of the transplanted cells.

The inner limiting membrane/neural fiber layer (ILM/NFL) of the eye acts as a physical barrier to the successful engraftment of donor RGCs into the GCL layer of the retina (53). Previously, the use of collagenase to digest ILM (54) or mechanical peeling of the ILM (55, 56) has been shown to be deleterious to the retinal structure. We performed intravitreal injections of 0.0001% Pronase E (n=5) 4-weeks prior to cell transplantation to digest the ILM and enhance cell engraftment. However, the use of Pronase E induced cataract formation and inflammation as observed by cSLO imaging (FIG. 22) and did not increase the efficiency of transplantation. We observed that C57BL/6J mice that received hiPSC-RGCs without Pronase E had a significant number of successfully transplanted cells. Furthermore, there is a greater probability that transplanted RGCs will extend their dendrites into the IPL and towards the optic nerve when the ILM layer of the host is intact (21, 25). This may be due to the presence of laminin in ILM that functions as a neuritogenic signal and growth substrate (57, 58).

Another factor that may influence the success of the transplantation is the immunological response mediated by the host. In terms of transplantation sites, the vitreous cavity of the eye is considered to be relatively immune-privileged. However, since we did not immune suppress our mice before injection and the hiPSC-RGCs are xenografts in mice, the eventual number of donor cells that survive the transplantation may depend on the response facilitated by the resident innate neuroinflammatory cells such as retinal macroglia (astrocytes and Müller glia) and microglia. To this end, we stained for the microglia-specific marker (Iba1) in the retinal sections transplanted with hiPSC-RGCs and did not detect the presence of microglia (Data not shown). Additionally, in a human clinical trial, the chances of immunologic rejection would be considerably reduced if autologous patient-derived iPSC-RGCs were used for transplantation.

In recent years, intercellular material transfer between donor and host cells has confounded the interpretation of transplantation studies (59-63).

Transplanted hiPSCs successfully integrated, produced axonal and elaborate dendritic connections in the host retinas, and were capable of generating light responses. Responses could be detected at moderate intensities (1e5 photon s⁻¹ μm⁻²). However, most of the responses were observed at 10-100 times brighter intensities. One of the reasons for the lower light sensitivity appears to be the reduced ability of these cells to fire upon repeated stimuli. To some degree, this can be compensated by increasing flash intensity and light-dependent membrane depolarization. The observed decrease in light sensitivity cannot be explained by photoreceptor bleaching alone; brighter flashes delivered later during the experiment produced larger depolarizations (suggesting a more powerful signal originating from photoreceptors) but a smaller increase in the firing rate compared to earlier, dimmer flashes. Accordingly, large depolarizations caused by current injections through patch-pipettes resulted in smaller than expected increases in the firing rate when performed after repeated light stimuli. It appears that hiPSCs may require a longer resting time to recover their ability to increase their firing rate in response to depolarizing stimuli. Partial restoration of this ability was observed when cells were rested in the dark for at least 30 seconds.

When designing a cell replacement strategy, the unique microenvironment present within aged glaucomatous host retinas must also be considered. hiPSC-RGC injections in mouse models of N-Methyl-D-Aspartate (NMDA) by following published protocols that recapitulate critical aspects of the glaucomatous disease model, induced RGC loss and optic nerve crush model of optic neuropathy (64-67). Thus far our intravitreal injection technique results in the successful detection of eGFP+hiPSC-RGCs in NMDA—(n=10/10) and optic nerve crush (n=7/8) models of glaucoma (Data not shown). The data indicates that glaucomatous retinas exhibit an increased ability to readily accept donor hiPSC-RGCs compared to wild type adult retinas.

In summary, we have shown that the two-step hiPSC differentiation protocol disclosed herein consistently generates a large population of pure RGCs reported to date. Furthermore, this method does not employ any additional purification step or gene modification as commonly required by other differentiation protocols. Intravitreal injections of hiPSC-RGCs into the vitreous cavity of adult wild type C57BL/6J mice resulted in a successful transplantation rate of about 94% and an average number of donor hiPSC-RGCs that survived was calculated to be 672 (range 230 to 1535). In relation to other groups that are also trying to regenerate RGCs via transplantation, their successful transplantation rate was reported as 10% (16, 21) and >65% (24). The transplantation efficiency in these studies ranged from 50 to >2,000 GFP+RGCs (21) and 0.5-5% (24), respectively.

The transplanted cells disclosed herein were localized within the ganglion cell layer and expressed RGC-specific markers within the host retinas. Furthermore, the transplanted hiPSC-RGCs exhibited similar morphology and functional activities as the endogenous murine RGCs. This data helps provide the necessary framework required for extending induced pluripotent stem cell studies beyond the pre-clinical stages of development and into human clinical trials. This novel strategy will help alleviate a significant emotional and financial burden endured by glaucoma patients and society.

### REFERENCES FOR EXAMPLE 6

1. Janssen S F, Gorgels T G, Ramdas W D, Klaver C C, van Duijn C M, Jansonius N M, et al. The vast complexity of primary open angle glaucoma: disease genes, risks, molecular mechanisms and pathobiology. Prog Retin Eye Res. 2013; 37:31-67.
2. Weinreb R N, Leung C K, Crowston J G, Medeiros F A, Friedman D S, Wiggs J L, et al. Primary open-angle glaucoma. Nature Reviews Disease Primers. 2016; 2:16067.
3. Tham Y, Li X, Wong T Y, Quigley H A, Aung T, Cheng C. Global prevalence of glaucoma and projections of glaucoma burden through 2040: a systematic review and meta-analysis. Ophthalmology. 2014; 121(11):2081-90.
4. Zhang N, Wang J, Li Y, Jiang B. Prevalence of primary open angle glaucoma in the last 20 years: a meta-analysis and systematic review. Scientific Reports. 2021; 11(1):13762.
5. Cook C, Foster P. Epidemiology of glaucoma: what's new? Canadian Journal of Ophthalmology. 2012; 47(3):223-6.
6. Quigley H A, Broman A T. The number of people with glaucoma worldwide in 2010 and 2020. Br J Ophthalmol. 2006; 90(3):262-7.
7. Rein D B, Zhang P, Wirth K E, Lee P P, Hoerger T J, McCall N, et al. The economic burden of major adult visual disorders in the United States. Arch Ophthalmol. 2006; 124(12):1754-60.
8. Charlson E S, Sankar P S, Miller-Ellis E, Regina M, Fertig R, Salinas J, et al. The Primary Open-Angle African American Glaucoma Genetics Study: Baseline Demographics. Ophthalmology. 2015; 122(4):711-20.
9. Salowe R, Salinas J, Farbman N H, Mohammed A, Warren J Z, Rhodes A, et al. Primary Open-Angle Glaucoma in Individuals of African Descent: A Review of Risk Factors. J Clin Exp Ophthalmol. 2015 August; 6(4):450. doi: 10.4172/2155,9570.1000450. Epub 2015 Jul. 31.
10. Kapetanakis V V, Chan M P, Foster P J, Cook D G, Owen C G, Rudnicka A R. Global variations and time trends in the prevalence of primary open angle glaucoma (POAG): a systematic review and meta-analysis. Br J Ophthalmol. 2016; 100(1):86-93.
11. Danford I D, Verkuil L D, Choi D J, Collins D W, Gudiseva H V, Uyhazi K E, et al. Characterizing the “POAGome”: A bioinformatics-driven approach to primary open-angle glaucoma. Prog Retin Eye Res. 2017; 58:89-114.
12. Heavner W, Pevny L. Eye development and retinogenesis. Cold Spring Harbor perspectives in biology. 2012; 4 (12):a008391.
13. Liu Y, Allingham R R. Major review: Molecular genetics of primary open-angle glaucoma. Exp Eye Res. 2017; 160:62-84.
14. Sommer A, Tielsch J M, Katz J, Quigley H A, Gottsch J D, Javitt J, et al. Relationship between intraocular pressure and primary open angle glaucoma among white and black Americans: the Baltimore Eye Survey. Arch Ophthalmol. 1991; 109(8):1090-5.
15. Cohen L P, Pasquale L R. Clinical characteristics and current treatment of glaucoma. Cold Spring Harbor perspectives in medicine. 2014; 4 (6):a017236.
16. Hertz J, Qu B, Hu Y, Patel R D, Valenzuela D A, Goldberg J L. Survival and integration of developing and progenitor-derived retinal ganglion cells following transplantation. Cell Transplant. 2014; 23(7):855-72.
17. Jagatha B, Divya M S, Sanalkumar R, Indulekha C L, Vidyanand S, Divya T S, et al. In vitro differentiation of retinal ganglion-like cells from embryonic stem cell derived neural progenitors. Biochem Biophys Res Commun. 2009; 380(2):230-5.
18. Divya M S, Rasheed V A, Schmidt T, Lalitha S, Hattar S, James J. Intraocular injection of ES cell-derived neural progenitors improve visual function in retinal ganglion cell-depleted mouse models. Frontiers in cellular neuroscience. 2017; 11:295.
19. Cho J, Mao C, Klein W H. Adult mice transplanted with embryonic retinal progenitor cells: new approach for repairing damaged optic nerves. Molecular vision. 2012; 18:2658.
20. Scruggs B A, Jiao C, Cranston C M, Kaalberg E, Wang K, Russell S R, et al. Optimizing Donor Cellular Dissociation and Subretinal Injection Parameters for Stem Cell-Based Treatments. Stem cells translational medicine.
21. Venugopalan P, Wang Y, Nguyen T, Huang A, Müller K J, Goldberg J L. Transplanted neurons integrate into adult retinas and respond to light. Nature communications. 2016; 7:10472.
22. Miltner A M, La Torre A. Retinal Ganglion Cell Replacement: Current Status and Challenges Ahead. Developmental Dynamics. 2019; 248(1):118-28.
23. Suen H C, Qian Y, Liao J, Luk C S, Lee W T, Ng J K W, et al. Transplantation of Retinal Ganglion Cells Derived from Male Germline Stem Cell as a Potential Treatment to Glaucoma. Stem Cells and Development. 2019; 28(20):1365-75.
24. Oswald J, Kegeles E, Minelli T, Volchkov P, Baranov P. Transplantation of miPSC/mESC-derived retinal ganglion cells into healthy and glaucomatous retinas. Mol Ther Methods Clin Dev. 2021 Mar. 10; 21:180-98.
25. Zhang K Y, Aguzzi E A, Johnson T V. Retinal Ganglion Cell Transplantation: Approaches for Overcoming Challenges to Functional Integration. Cells. 2021 Jun. 8; 10(6):1426. doi: 10.3390/cells10061426.
26. Chavali V R M, Haider N, Rathi S, Vrathasha V, Alapati T, He J, et al. Dual SMAD inhibition and Wnt inhibition enable efficient and reproducible differentiations of induced pluripotent stem cells into retinal ganglion cells. Scientific Reports. 2020; 10(1):11828.
27. Teotia P, Chopra D A, Dravid S M, Van Hook M J, Qiu F, Morrison J, et al. Generation of functional human retinal ganglion cells with target specificity from pluripotent stem cells by chemically defined recapitulation of developmental mechanism. Stem Cells. 2017; 35(3):572-85.
28. Pashos E E, Park Y, Wang X, Raghavan A, Yang W, Abbey D, et al. Large, Diverse Population Cohorts of hiPSCs and Derived Hepatocyte-like Cells Reveal Functional Genetic Variation at Blood Lipid-Associated Loci. Cell Stem Cell. 2017 Apr. 6; 20(4):558,570.e10.
29. Gudiseva H V, Vrathasha V, He J, Bungatavula D, O'Brien J M, Chavali V R M. Single Cell Sequencing of Induced Pluripotent Stem Cell Derived Retinal Ganglion Cells (iPSC-RGC) Reveals Distinct Molecular Signatures and RGC Subtypes. Genes. 2021; 12 (12).
30. Ross A G, McDougald D S, Khan R S, Duong T T, Dine K E, Aravand P, et al. Rescue of retinal ganglion cells in optic nerve injury using cell-selective AAV mediated delivery of SIRT1. Gene Ther. 2021; 28(5):256-64.
31. Clark P J, Evans F C. Distance to Nearest Neighbor as a Measure of Spatial Relationships in Populations. Ecology. 1954; 35(4):445-53.
32. Keeley P W, Eglen S J, Reese B E. From random to regular: Variation in the patterning of retinal mosaics*. J Comp Neurol. 2020; 528(13):2135-60.
33. Cressie N. Statistics for spatial data. John Wiley & Sons; 2015.
34. Drager U C, Olsen J F. Ganglion cell distribution in the retina of the mouse. Invest Ophthalmol Vis Sci. 1981; 20(3):285-93.
35. Didonna A, Opal P. The role of neurofilament aggregation in neurodegeneration: lessons from rare inherited neurological disorders. Molecular Neurodegeneration. 2019; 14(1):19.
36. Tansley K. The gecko retina. Vision Res. 1964; 4 (1-2):33-IN14.
37. Banin E, Obolensky A, Idelson M, Hemo I, Reinhardtz E, Pikarsky E, et al. Retinal incorporation and differentiation of neural precursors derived from human embryonic stem cells. Stem Cells. 2006 February; 24(2):246-57.
38. Osakada F, Ikeda H, Mandai M, Wataya T, Watanabe K, Yoshimura N, et al. Toward the generation of rod and cone photoreceptors from mouse, monkey and human embryonic stem cells. Nat Biotechnol. 2008 February; 26(2):215-24.
39. Lamba D A, Karl M O, Ware C B, Reh T A. Efficient generation of retinal progenitor cells from human embryonic stem cells. Proc Natl Acad Sci USA. 2006 Aug. 22; 103(34):12769-74.
40. Ikeda H, Osakada F, Watanabe K, Mizuseki K, Haraguchi T, Miyoshi H, et al. Generation of Rx+/Pax6+ neural retinal precursors from embryonic stem cells. Proc Natl Acad Sci USA. 2005 Aug. 9; 102(32):11331-6.
41. Meno C, Ito Y, Saijoh Y, Matsuda Y, Tashiro K, Kuhara S, et al. Two closely-related left-right asymmetrically expressed genes, lefty-1 and lefty-2: their distinct expression domains, chromosomal linkage and direct neuralizing activity in Xenopus embryos. Genes Cells. 1997 August; 2(8):513-24.
42. del Barco Barrantes I, Davidson G, Gröne H J, Westphal H, Niehrs C. Dkk1 and noggin cooperate in mammalian head induction. Genes Dev. 2003 Sep. 15; 17(18):2239-44.
43. Davis A A, Matzuk M M, Reh T A. Activin A promotes progenitor differentiation into photoreceptors in rodent retina. Mol Cell Neurosci. 2000 January; 15(1):11-21.
44. Aoki H, Hara A, Niwa M, Motohashi T, Suzuki T, Kunisada T. An in vitro mouse model for retinal ganglion cell replacement therapy using eye-like structures differentiated from E S cells. Exp Eye Res. 2007 May; 84(5):868-75.
45. Meyer J S, Shearer R L, Capowski E E, Wright L S, Wallace K A, McMillan E L, et al. Modeling early retinal development with human embryonic and induced pluripotent stem cells. Proc Natl Acad Sci USA. 2009 Sep. 29; 106(39):16698-703.
46. Meyer J S, Howden S E, Wallace K A, Verhoeven A D, Wright L S, Capowski E E, et al. Optic vesicle-like structures derived from human pluripotent stem cells facilitate a customized approach to retinal disease treatment. Stem Cells. 2011; 29(8):1206-18.
47. Nakano T, Ando S, Takata N, Kawada M, Muguruma K, Sekiguchi K, et al. Self-formation of optic cups and storable stratified neural retina from human ESCs. Cell Stem Cell. 2012 Jun. 14; 10(6):771-85.
48. Eiraku M, Takata N, Ishibashi H, Kawada M, Sakakura E, Okuda S, et al. Self-organizing optic-cup morphogenesis in three-dimensional culture. Nature. 2011 Apr. 7; 472(7341):51-6.
49. Singh R, Shen W, Kuai D, Martin J M, Guo X, Smith M A, et al. iPS cell modeling of Best disease: insights into the pathophysiology of an inherited macular degeneration. Hum Mol Genet. 2012; 22(3):593-607.
50. Chamling X, Sluch V M, Zack D J. The potential of human stem cells for the study and treatment of glaucoma. Invest Ophthalmol Vis Sci. 2016; 57 (5):ORSFil-6.
51. Varela-Fernández R, Díaz-Tomé V, Luaces-Rodríguez A, Conde-Penedo A, García-Otero X, Luzardo-Álvarez A, et al. Drug Delivery to the Posterior Segment of the Eye: Biopharmaceutic and Pharmacokinetic Considerations. Pharmaceutics. 2020; 12(3):269.
52. Johnson T V, Martin K R. Development and characterization of an adult retinal explant organotypic tissue culture system as an in vitro intraocular stem cell transplantation model. Invest Ophthalmol Vis Sci. 2008 August; 49(8):3503-12.
53. Zhang K Y, Tuffy C, Mertz J L, Quillen S, Wechsler L, Quigley H A, et al. Role of the Internal Limiting Membrane in Structural Engraftment and Topographic Spacing of Transplanted Human Stem Cell-Derived Retinal Ganglion Cells. Stem Cell Reports. 2021 Jan. 12; 16(1):149-67.
54. Dalkara D, Kolstad K D, Caporale N, Visel M, Klimczak R R, Schaffer D V, et al. Inner Limiting Membrane Barriers to AAV-mediated Retinal Transduction From the Vitreous. Molecular Therapy. 2009; 17(12):2096-102.
55. Takahashi K, Igarashi T, Miyake K, Kobayashi M, Yaguchi C, Iijima O, et al. Improved Intravitreal AAV-Mediated Inner Retinal Gene Transduction after Surgical Internal Limiting Membrane Peeling in Cynomolgus Monkeys. Molecular Therapy. 2017; 25(1):296-302.
56. Teo K Y C, Lee S Y, Barathi A V, Tun S B B, Tan L, Constable I J. Surgical Removal of Internal Limiting Membrane and Layering of AAV Vector on the Retina Under Air Enhances Gene Transfection in a Nonhuman Primate. Invest Ophthalmol Vis Sci. 2018; 59(8):3574-83.
57. Randlett O, Poggi L, Zolessi F R, Harris W A. The oriented emergence of axons from retinal ganglion cells is directed by laminin contact in vivo. Neuron. 2011 Apr. 28; 70(2):266-80.
58. Riccomagno M M, Sun L O, Brady C M, Alexandropoulos K, Seo S, Kurokawa M, et al. Cas adaptor proteins organize the retinal ganglion cell layer downstream of integrin signaling. Neuron. 2014 Feb. 19; 81(4):779-86.
59. Nickerson P E B, Ortin-Martinez A, Wallace V A. Material exchange in photoreceptor transplantation: Updating our understanding of donor/host communication and the future of cell engraftment science. Frontiers in Neural Circuits. 2018; 12.
60. Pearson R A, Gonzalez-Cordero A, West E L, Ribeiro J R, Aghaizu N, Goh D, et al. Donor and host photoreceptors engage in material transfer following transplantation of post-mitotic photoreceptor precursors. Nature Communications. 2016; 7.
61. Waldron P V, Di Marco F, Kruczek K, Ribeiro J, Graca A B, Hippert C, et al. Transplanted Donor- or Stem Cell-Derived Cone Photoreceptors Can Both Integrate and Undergo Material Transfer in an Environment-Dependent Manner. Stem Cell Reports. 2018; 10(2):406-21.
62. Singh M S, Balmer J, Barnard A R, Aslam S A, Moralli D, Green C M, et al. Transplanted photoreceptor precursors transfer proteins to host photoreceptors by a mechanism of cytoplasmic fusion. Nature Communications. 2016; 7.
63. Santos-Ferreira T, Llonch S, Borsch O, Postel K, Haas J, Ader M. Retinal transplantation of photoreceptors results in donor-host cytoplasmic exchange. Nature Communications. 2016; 7(1):13028.
64. Lam T T, Abler A S, Kwong J M, Tso M O. N-methyl-D-aspartate (NMDA)â€ “induced apoptosis in rat retina. Invest Ophthalmol Vis Sci. 1999; 40(10):2391-7.
65. Honda S, Namekata K, Kimura A, Guo X, Harada C, Murakami A, et al. Survival of Alpha and Intrinsically Photosensitive Retinal Ganglion Cells in NMDA-Induced Neurotoxicity and a Mouse Model of Normal Tension Glaucoma. Invest Ophthalmol Vis Sci. 2019; 60(12):3696-707.
66. Khan R, Chen Y, Cwanger A, Song Y, Dunaief J, Kim S, et al. Dexras1 Mediates Retinal Ganglion Cell Loss Induced by NMDA Excitotoxicity. Invest Ophthalmol Vis Sci. 2013; 54(15):1414.
67. Chen Y, Khan R S, Cwanger A, Song Y, Steenstra C, Bang S, et al. Dexras1, a Small GTPase, Is Required for Glutamate-NMDA Neurotoxicity. J Neurosci. 2013; 33(8):3582-7.
68. Duong T T, Vasireddy V, Ramachandran P, Herrera P S, Leo L, Merkel C, et al. Use of induced pluripotent stem cell models to probe the pathogenesis of Choroideremia and to develop a potential treatment. Stem cell research. 2018; 27:140-50.
69. Sullivan S K, Mills J A, Koukouritaki S B, Vo K K, Lyde R B, Paluru P, et al. High-level transgene expression in induced pluripotent stem cell-derived megakaryocytes: correction of Glanzmann thrombasthenia. Blood. 2014; 123(5):753-7.
70. Maguire J A, Gagne A, Mills J A, Gadue P, French D L. Generation of human control iPS cell line CHOPWT9 from healthy adult peripheral blood mononuclear cells. Stem Cell Research. 2016; 16(1):14-6.
71. Espuny-Camacho I, Michelsen K A, Gall D, Linaro D, Hasche A, Bonnefont J, et al. Pyramidal neurons derived from human pluripotent stem cells integrate efficiently into mouse brain circuits in vivo. Neuron. 2013; 77(3):440-56.
72. McDougald D S, Dine K E, Zezulin A U, Bennett J, Shindler K S. SIRT1 and NRF2 Gene Transfer Mediate Distinct Neuroprotective Effects Upon Retinal Ganglion Cell Survival and Function in Experimental Optic Neuritis. Invest Ophthalmol Vis Sci. 2018; 59(3):1212-20.
73. Chaffiol A, Caplette R, Jaillard C, Brazhnikova E, Desrosiers M, Dubus E, et al. A New Promoter Allows Optogenetic Vision Restoration with Enhanced Sensitivity in Macaque Retina. Mol Ther. 2017 Nov. 1; 25(11):2546-60.
74. Costello F, Coupland S, Hodge W, Lorello G R, Koroluk J, Pan Y I, et al. Quantifying axonal loss after optic neuritis with optical coherence tomography. Ann Neurol. 2006 June; 59(6):963-9.
75. Berg S, Kutra D, Kroeger T, Straehle C N, Kausler B X, Haubold C, et al. Ilastik: Interactive Machine Learning for (Bio)image Analysis. Nat Methods. 2019 December; 16(12):1226-32.

### Example 7

### RGC Cell Replacement Therapy for the Human Eye

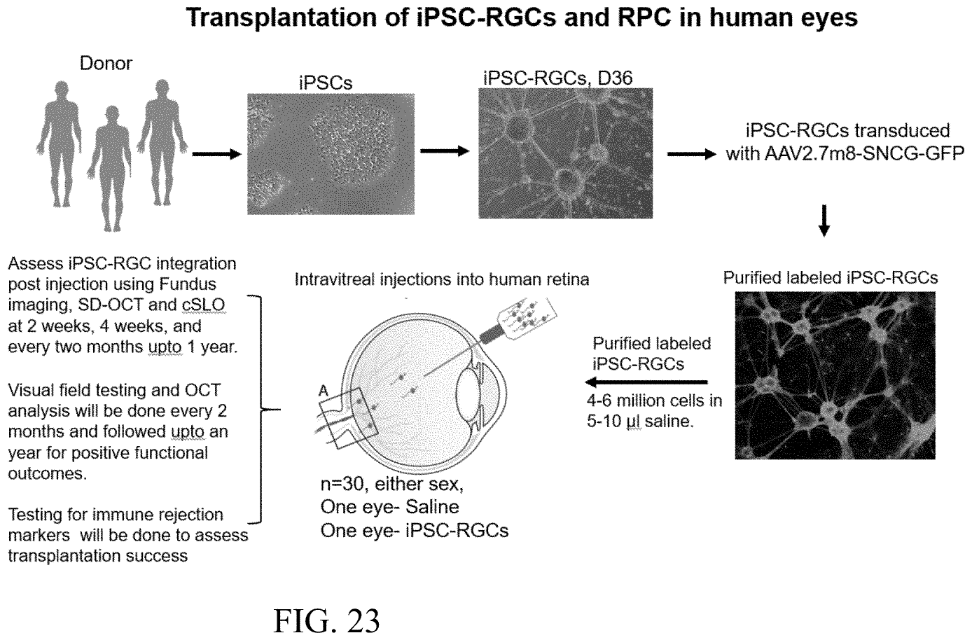

The information herein above can be applied clinically to patients for therapeutic intervention as part of a cell replacement therapy. One embodiment of the cell replacement therapy is shown in FIG. 23. A preferred embodiment of the invention comprises clinical application of the RGCs recited herein to a patient. This can occur after a patient arrives in the clinic and presents with symptoms of a progressive optic neuropathy. In certain embodiments, the progressive optic neuropathy is glaucoma. Purified iPSC-RGCs can be injected intravitreally. In certain embodiments RGCs are harvested at Day 32-35 of culture. In other embodiments, the RGCs are induced from stem cells obtained from the patient. In certain embodiments, a 30-34 gauge needle is used to administer the iPSC-RGCs. Integration of iPSC-RGCs into human retina can be evaluated using ERGs and Optokinetic Response (OKR). In certain embodiments, administration of the iPSC-RGCs results in a reduction in progressive optic neuropathy symptoms. After administration, patients may be assessed using Fundus imaging, SD-OCT, and/or cSLO every 2 weeks, every 4 weeks, and/or every two month. This assessment may continue for 1 year or longer. In certain embodiments, visual field testing and OCT analysis will be done for positive functional outcomes. Such testing may occur every 2 months for 1 year or longer. Testing for immune rejection markers can also be done to assess transplantation success.

In certain embodiments, personalized treatments are administered by deriving the iPSCs from the glaucoma patient. These iPSCs are then treated and the purified iPSC-RGCs are then administered to the back to the patient. In certain embodiments the iPSCs are derived from the patients blood using peripheral blood mononuclear cells (PBMNCs) or using skin biopsies to obtain and culture keratinocytes. Other sources of stem cells, such as fat cells, can be used.

In a different approach, a control iPSC is obtained from a source other than the patient. In certain embodiments, the control iPSC has no SNPs that are associated with primary open angle glaucoma, age related macular degeneration or retinal degenerations. These control cells may be cultured in batches, thereby generating retinal progenitor cell (RPC) libraries. These RPC libraries may be frozen. In certain embodiments the RPC libraries are frozen at Day 10, 12, 15 or 18. These RPCs are unfrozen prior to differentiation into RGCs using the protocol described above. Mature iPSC-RGCs may then be administered via intravitreal injection into a patient's retina for cell replacement therapy.

RPC may be generated from the protocol described above. These RPCs can used as retinal precursors. The RPCs may be injected intravitreally to differentiate into photoreceptors to treat their loss in patients with retinal degeneration as cell-based therapy.

The iPSC-RGCs can optionally be labeled prior to administration, e.g., with a fluorescent label or channel rhodopsin proteins for optogenetic treatment approaches. iPSC-RGCs can be labeled by transduction with an AAV virus as described in Example 2.

The compositions and methods disclosed herein provide the means to generate and produce homogeneous populations of retinal ganglion cells that can be used to advantage for the treatment of various ocular disorders.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

### Embodiments

1. A method of preparing retinal progenitor cells and retinal ganglion cells by directed differentiation of induced pluripotent stem cells (iPSCs) in a defined chemical medium, comprising,
a) culturing said iPSC in iPSC initiation medium;
b) transferring said iPSC of step a) to retinal progenitor cell (RPC) culture medium for transdifferentiation into RPCs;
c) culturing said RPCs of step b) in retinal ganglion cell (RGC) culture medium for transdifferentiation into RGCs; thereby providing a population of RGCs suitable for in vitro studies and for transplantation into the eye.

2. The method of embodiment 1, wherein said iPSCs are cultured in a 37° C., 5% CO₂ and 5% O₂ incubator and the iPSC initiation medium comprises one of:
i) 80% HES/20% MEF-CM+20 ng/ml bFGF+4 μM Y27632;
ii) 100% HES+20 ng/ml bFGF+4 μM Y27632; or
iii) 100% StemMACS™ iPS Brew-Xenofree (XF)+20 ng/ml bFGF+4 μM Y27632.

3. The method of any of the preceding embodiments, wherein the RPC culture medium comprises RPC induction media, with 0.1 μM LDN193189, 10 μM SB431542, 2 μM XAV939, 10 mM Nicotinamide, 10 ng/ml IGF1, 1.5 μM CHIR99021 and 10 ng/ml bFGF reagents.

4. The method of any one of the preceding embodiments, wherein the RGC culture medium comprises RGC induction media, 3 μM DAPT, 250 ng/ml sonic hedgehog (SHH) and 100 ng/ml FGF8 or smoothened agonist (SAG).

5. The method of any of the preceding embodiments comprising isolating said RGCs.

6. The method of embodiment 5, wherein said RGCs express BRN3A, BRN3B, TUBB3, CD90, MAP2, TUJ1, RBPMS and TUJ1 markers.

7. The method of any of the preceding embodiments wherein said RGC population is obtained after between 30 to 45 days in culture from iPSCs.

8. A method for the production of retinal progenitor cells (RPCs), comprising;
a) culturing iPSCs on 0.1% gelatinized plates containing irradiated MEFs until cells achieved approximately 75% confluence;
b) removing said MEFs and plating remaining iPSCs onto plates containing 1:100 diluted growth factor reduced Matrigel and iPSC: MEF-conditioned medium (80:20)+20 ng/mL of bFGF and 5 ng/ml of stable bFGF until reaching 100% confluence;
c) replacing iPSC: MEF-conditioned media with RPC induction media comprising DMEM/F12 (50:50), 1% P/S, 1% Glut, 1% NEAA, 0.1 mM 2-ME, 2% B27 supplement (w/o vitamin A), 1% N2 supplement, containing effective amounts of a Wnt inhibitor, a TGFβ inhibitor and a BMP inhibitor, 10 mM nicotinamide, and 10 ng/mL IGF1 and culturing cells for 4 days with daily media changes;
d) replacing the culture media of step c) with RPC induction media containing an effective amount of a Wnt inhibitor, a TGFβ inhibitor and a BMP inhibitor, 10 ng/mL IGF1, and 10 ng/mL bFGF on day 5 and culturing said cells for 12 days with daily media changes;
e) replacing RPC induction media with RGC differentiation media comprising 0.1 μM LDN193198, 10 μM SB431542, 2 μM XAV939, 1.5 μM CHIR99021, 10 ng/ml IGF1 and 10 ng/ml bFGF on day 16 of differentiation culturing said cells until Day 23, upon which RGC differentiation occurred. thereby producing a population of RGCs suitable for transplantation into the eye.

9. The method of any of the preceding embodiments wherein said Wnt inhibitor is XAV939, said TGF-β inhibitor is SB431542 and said BMP inhibitor is LDN193189.

10. The method of any of the preceding embodiments, wherein the iPSCs are from stem cells selected from the group consisting of bone marrow stem cells (BMS), cord blood stem cells, amniotic fluid stem cells, fat stem cells, retinal stem cells (RSCs), keratinocyte stem cells, intraretinal Müller glial cells, embryonic stem cells (ESCs), corneal endothelial cells and somatic cell nuclear transfer cells (SCNTCs).

11. The method of embodiment 8, wherein the Wnt signaling pathway activator is one or more selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16b, substance increasing p3-catenin levels; lithium, LiCl, bivalent zinc, BIO (6-bromoindirubin-3′-oxime), SB216763, SB415286, CHIR99021, QS11 hydrate, TWS119, Kenpaullone, alsterpaullone, indirubin-3′-oxime, TDZD-8 and Ro 318220 methanesulfonate salt; Axin inhibitors; APC (adenomatous polyposis coli) inhibitors; norrin and R-spondin 2.

12. The method of embodiment 8, further comprising the step of determining whether the retinal progenitor cells are differentiated into the mature retinal ganglion cells.

13. The method of embodiment 12, wherein the step of determining whether the retinal progenitor cells are differentiated into the mature retinal ganglion cells is performed by measuring mRNA or protein expression levels of one or more genes selected from the group consisting of SOX2, RAX, PAX6, SIX6, SIX3, VSX2 and LHX2.

14. The method of embodiment 1, wherein the mature retinal ganglion cells are present at from about 60% to about 95% or more of total cells after the culturing of step

15. The method of embodiment 1, wherein said RGCs comprise an AAV vector expressing a transgene of interest.

16. The method of embodiment 1, where in RGCs are differentiated from iPSCs without genetic manipulation using CRISPR gene editing methodologies.

17. The method of embodiment 1, where in iPSCs are differentiated into RGCs from normal and disease patients with ocular pathologies selected from glaucoma, age-related macular degeneration (AMD) or any type of retinal degeneration, a disorder related to an increase in intraocular pressure (IOP), a disorder related to neuroprotection, or diabetic retinopathy.

18. A method for the treatment of an ocular disorder, comprising administering a therapeutically effective amount of a population of purified RGCs as embodied in embodiment 1, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

19. A method for the treatment of an ocular disorder, comprising administering a therapeutically effective amount of a population of purified RPCs as embodied in embodiment 8, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

20. The method according to embodiment 18 or embodiment 19, wherein the disorder is glaucoma.

21. The method of embodiment 18 or embodiment 19, wherein the disorder is related to an increase in intraocular pressure (IOP).

22. The method of embodiment 18 or embodiment 19, wherein the age-related macular degeneration is wet age-related macular degeneration.

23. The method of embodiment 18 or embodiment 19, wherein the host is a human.

24 The method of embodiment 18 or embodiment 19, wherein the cells are administered via intravitreal, intrastromal, intracameral, sub-tenon, sub-retinal, retro-bulbar, peribulbar, suprachoroidal, choroidal, subchoroidal, conjunctival, subconjunctival, epi scleral, posterior juxtascleral, circumcorneal, optic nerve or tear duct injection.

25. The method of embodiment 22, wherein the cells are administered via intravitreal injection.

26. The method of embodiment 22, wherein the compound is administered via subretinal injection.

27. The method of embodiment 22, wherein the cells described in embodiment 18 and embodiment 19 are administered through routes in embodiment 24 either before optic nerve injury through optic nerve crush or via delivery to the optic nerve.

28. The method of any one of the preceding embodiments, wherein the RGC maturation medium comprises 30 ng/ml to 50 ng/ml BDNF.

29. A method for the treatment of an ocular disorder, comprising administering a therapeutically effective amount of a population of purified RGCs using immunopanning, flowcytometry and cell sorting with RGC specific surface antibodies, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

30. The method of embodiment 29, wherein CD90+RGCs are immunopurified via MACS sorting with CD90.2 magnetic beads.

31. An isolated population of RGC prepared by the method of embodiment 1.

32. An isolated population of RPC prepared by the method of embodiment 8.

What is claimed is:

1. A method of preparing retinal progenitor cells and retinal ganglion cells by directed differentiation of induced pluripotent stem cells (iPSCs) in a defined chemical medium, comprising,
a) culturing said iPSC in iPSC initiation medium;
b) transferring said iPSC of step a) to retinal progenitor cell (RPC) culture medium for transdifferentiation into RPCs;
c) culturing said RPCs of step b) in retinal ganglion cell (RGC) culture medium for transdifferentiation into RGCs; thereby providing a population of RGCs suitable for in vitro studies and for transplantation into the eye.

2. The method of claim 1, wherein said iPSCs are cultured in a 37° C., 5% CO₂ and 5% O₂ incubator and the iPSC initiation medium comprises one of:
i) 80% HES/20% MEF-CM+20 ng/ml bFGF+4 μM Y27632;
ii) 100% HES+20 ng/ml bFGF+4 μM Y27632; or
iii) 100% StemMACS™ iPS Brew-Xenofree (XF)+20 ng/ml bFGF+4 μM Y27632.

3. The method of claim 1, wherein the RPC culture medium comprises RPC induction media, with 0.1 μM LDN193189, 10 μM SB431542, 2 μM XAV939, 10 mM Nicotinamide, 10 ng/ml IGF1, 1.5 μM CHIR99021 and 10 ng/ml bFGF reagents.

4. The method of claim 1, wherein the RGC culture medium comprises RGC induction media, 3 μM DAPT, 250 ng/ml sonic hedgehog (SHH) and 100 ng/ml FGF8 or smoothened agonist (SAG).

5. The method of claim 1, further comprising isolating said RGCs, wherein said RGCs express BRN3A, BRN3B, TUBB3, CD90, MAP2, TUJ1, RBPMS and TUJ1 markers.

6. The method of claim 1 wherein said RGC population is obtained after between 30 to 45 days in culture from iPSCs.

7. A method for the production of retinal progenitor cells (RPCs), comprising;
a) culturing iPSCs on 0.1% gelatinized plates containing irradiated MEFs until cells achieved approximately 75% confluence;
b) removing said MEFs and plating remaining iPSCs onto plates containing 1:100 diluted growth factor reduced Matrigel and iPSC: MEF-conditioned medium (80:20)+20 ng/mL of bFGF and 5 ng/ml of stable bFGF until reaching 100% confluence;
c) replacing iPSC: MEF-conditioned media with RPC induction media comprising DMEM/F12 (50:50), 1% P/S, 1% Glut, 1% NEAA, 0.1 mM 2-ME, 2% B27 supplement (w/o vitamin A), 1% N2 supplement, containing effective amounts of a Wnt inhibitor, a TGFβ inhibitor and a BMP inhibitor, 10 mM nicotinamide, and 10 ng/mL IGF1 and culturing cells for 4 days with daily media changes;
d) replacing the culture media of step c) with RPC induction media containing an effective amount of a Wnt inhibitor, a TGFβ inhibitor and a BMP inhibitor, 10 ng/mL IGF1, and 10 ng/mL bFGF on day 5 and culturing said cells for 12 days with daily media changes;
e) replacing RPC induction media with RGC differentiation media comprising 0.1 μM LDN193198, 10 μM SB431542, 2 μM XAV939, 1.5 μM CHIR99021, 10 ng/ml IGF1 and 10 ng/ml bFGF on day 16 of differentiation culturing said cells until Day 23, upon which RGC differentiation occurred thereby producing a population of RGCs suitable for transplantation into the eye.

8. The method of claim 7, wherein the Wnt signaling pathway activator is one or more selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16b, substance increasing p3-catenin levels; lithium, LiCl, bivalent zinc, BIO (6-bromoindirubin-3′-oxime), SB216763, SB415286, CHIR99021, QS 11 hydrate, TWS 119, Kenpaullone, alsterpaullone, indirubin-3′-oxime, TDZD-8 and Ro 318220 methanesulfonate salt; Axin inhibitors; APC (adenomatous polyposis coli) inhibitors; norrin and R-spondin 2.

9. The method of claim 7, further comprising the step of determining whether the retinal progenitor cells are differentiated into the mature retinal ganglion cells.

10. The method of claim 1, wherein the mature retinal ganglion cells are present at from about 60% to about 95% or more of total cells after the culturing of step

11. The method of claim 1, where in iPSCs are differentiated into RGCs from normal and disease patients with ocular pathologies selected from glaucoma, age-related macular degeneration (AMD) or any type of retinal degeneration, a disorder related to an increase in intraocular pressure (IOP), a disorder related to neuroprotection, or diabetic retinopathy.

12. A method for the treatment of an ocular disorder, comprising administering a therapeutically effective amount of a population of purified RGCs as claimed in claim 1, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

13. A method for the treatment of an ocular disorder, comprising administering a therapeutically effective amount of a population of purified RPCs as claimed in claim 7, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

14. The method of claim 12, wherein the cells are administered via intravitreal, intrastromal, intracameral, sub-tenon, sub-retinal, retro-bulbar, peribulbar, suprachoroidal, choroidal, subchoroidal, conjunctival, subconjunctival, epi scleral, posterior juxtascleral, circumcorneal, optic nerve or tear duct injection.

15. The method of claim 13, wherein the cells are administered via intravitreal, intrastromal, intracameral, sub-tenon, sub-retinal, retro-bulbar, peribulbar, suprachoroidal, choroidal, subchoroidal, conjunctival, subconjunctival, epi scleral, posterior juxtascleral, circumcorneal, optic nerve or tear duct injection.

16. The method of claim 1, wherein the RGC maturation medium comprises 30 ng/ml to 50 ng/ml BDNF.

17. A method for the treatment of an ocular disorder, comprising administering a therapeutically effective amount of a population of purified RGCs using immunopanning, flowcytometry and cell sorting with RGC specific surface antibodies, in a pharmaceutically carrier to a host in need thereof wherein the disorder is selected from glaucoma, age-related macular degeneration (AMD), a disorder related to an increase in intraocular pressure (IOP), a disorder related to neuroprotection, or diabetic retinopathy.

18. The method of claim 17, wherein CD90+RGCs are immunopurified via MACS sorting with CD90.2 magnetic beads.

19. An isolated population of RGC prepared by the method of claim 1.

20. An isolated population of RPC prepared by the method of claim 7.
